SEQUENCE LISTING (41 sequences)

SEQ ID NO: 1 - DNA, 2211 bp, Artificial
  LP1-1 nucleotide sequence
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggcgacagagtcatcaccaccagcacccgaacctgggccctgcccacttacaacaaccatctctacaagcaaatctccagtgcttcaacgggggccagcaacgacaaccactacttcggctacagcaccccctgggggtattttgacttcaacagattccactgccacttctcaccacgtgactggcagcgactcatcaacaacaactggggattccggcctaagcgactcaacttcaaactcttcaacattcaggtcaaagaggttacggacaacaatggagtcaagaccatcgccaataaccttaccagcacggtccaggtcttcacggactcggagtaccagttgccgtacgtcctcggctctgcgcaccagggctgcctccctccgttcccggcggacgtgttcatgattccgcagtacggctacctaacgcttaacaatggcagccaggcagtgggacggtcatccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcctcacacaaagacgacaaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 2 - DNA, 2211 bp, Artificial
  LP1-11 nucleotide sequence
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggcgccgacggagtgggtagttcctcaggaaattggcattgcgattccacatggctgggcgacagagtcatcaccaccagcaccagaacctgggccctgcccacctacaacaaccacctctacaagcaaatctccagtgcttcaacgggggccagcaacgacaaccactacttcggctacagcaccccttgggggtattttgacttcaacagattccactgccacttttcaccacgtgactggcaaagactcatcaacaacaactggggattccgacccaagagactcaacttcaagctctttaacatccaggtcaaagaggttacggacaacaatggagtcaagaccatcgccaataaccttaccagcacggtccaggtcttcacggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcctcacacaaagacgacaaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 3 - DNA, 2214 bp, Artificial
  LP1-13 nucleotide sequence
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcagtcgccacaagagccagactcctcctcgggcatcggcaagacaggccagcagcccgctaaaaagagactcaattttggtcagactggcgactcagagtcagtccccgacccacaacctctcggagaacctccagcagcgccctctggtgtgggacctaatacaatggctgcaggcggtggcgcaccaatggcagacaataacgaaggtgccgatggagtgggtagttcctcgggaaattggcattgcgattcccaatggctgggggacagagtcatcaccaccagcacccgaacctggaccctgcccacctacaacaatcacctctacaagcaaatctccaacagcacatctggaggagccaccaacgacaacacctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaaccggcaacaacttccagtttacttacaccttcgaggacgtgcctttccacagcagctacgcgcacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 4 - DNA, 2211 bp, Artificial
  LP1-18 nucleotide sequence
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtagttcctcgggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcggagtaccagttgccgtacgtcctcggctctgcgcaccagggctgcctccctccgttcccggcggacgtgttcatgattcctcagtacgggtatctgacgcttaatgatggaagccaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacaaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 5 - DNA, 2211 bp, Artificial
  LP1-23 nucleotide sequence
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctccaagcgggtgacaatccgtacctgcggtataaccacgccgacgccgagtttcaggagcgtcttcaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgccaaaaagagactcaatttcggtcagactggcggcacagagtcagttccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccagtgcttcaacgggggccagcaacgacaaccactacttcggctacagcaccccctgggggtattttgacttcaaccgcttccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcggactatcagctcccgtacgtgctcgggtcggctcacgagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaacaactttaccttcagctacacctttgaggacgttcctttccacagcagctacgctcacagccagagtctggaccggctgatgaatcctctgattgaccagtacctgtactacttgtctcggactcaaacaacaggaggtacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcctcacacaaagacgacaaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 6 - DNA, 2211 bp, Artificial
  LP2-10 nucleotide sequence
atggctgccgatggttatcttccagattggctcgaggacactctctctgaaggaataagacagtggtggaagctcaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgccaaaaagagactcaatttcggtcagactggcggcacagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccagtgcttcaacgggggccagcaacgacaaccactacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctccgcccaccagggctgcctgcctccgttcccggcggacgtcttcatgattcctcagtacgggtacctgactctgaacaatggcagtcaggccgtgggacgctcctccttctactgcctggaatactttccttcgcagatgctgagaaccggcaacaacttccagtttacttacaccttcgaggacgtgcctttccacagcagctacgcgcacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacaaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctactagtgtggactttgctgttaatacagaaggcgtgtactctgaaccccaccccattggcacccgttacctcacccgtcccctgtaa

SEQ ID NO: 7 - DNA, 2211 bp, Artificial
  LP2-19 nucleotide sequence
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggccaagaagcgggttcttgaacctcttggtctggttgaggaaggcgctaagacggctcctggaaagaagaggcctgtagagcagtctcctcaggaaccggactcctccgcgggtattggcaaatcgggtgcacagcccgccaaaaagagactcaatttcggtcagactggcgactcagagtcagttccagaccctcaacctctcggagaacctccagcagcgccctctggtgtgggacctaatacaatggcttcaggtggtggcgcaccagtggcagacaataacgaaggtgccgatggagtgggtagttcctcgggaaattggcattgcgattcccaatggctgggcgacagagtcatcaccaccagcacccgaacctgggccctgcccacttacaacaaccatctctacaagcaaatctccagtgcttcaacgggggccagcaacgacaaccactacttcggctacagcaccccctgggggtattttgacttcaacagattccactgccacttctcaccacgtgactggcagcgactcatcaacaacaactggggattccggcctaagcgactcaacttcaaactcttcaacattcaggtcaaagaggttacggacaacaatggagtcaagaccatcgccaataaccttaccagcacggtccaggtcttcacggactcggagtaccagttgccgtacgtcctcggctctgcgcaccagggctgcctccctccgttcccggcggacgtgttcatgattcctcagtacggctacctaacgctcaacaatggcagccaggcagtgggacggtcatccttttactgcctggaatatttcccatcgcagatgctgagaaccggcaacaacttccagtttacttacaccttcgaggacgtgcctttccacagcagctacgcgcacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaatacaatctgaatggaagaaattcattggctaatcctggcatcgctatggcctcacacaaagacgacaaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 8 - DNA, 2208 bp, Artificial
  LP2-20 nucleotide sequence
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaacgcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 9 - DNA, 2208 bp, Artificial
  LP3-19 nucleotide sequence
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccgtcagctcgacagcggagacaacccgtacctcaagtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcggacaataacgagggcgccgacggagtgggtaattcctcgggaaattggcattgcgattccacatggctgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaacaactttaccttcagctacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtttggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 10 - DNA, 2214 bp, Artificial
  LP4-7 nucleotide sequence
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtagttcctcgggaaattggcattgcgattcccaatggctgggggacagagtcatcaccaccagcacccgaacctgggccctgcccacctacaacaaccacctctacaagcaaatctccaacagcacatctggaggagccaccaacgacaacacctacttcggctacagcaccccctgggggtattttgactttaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcggagtaccagttgccgtacgtcctcggctctgcgcaccagggctgcctccctccgttcccggcggacgtgttcatgattcctcagtacgggtacctgactctgaacaacggcagtcaggccgtgggccgttcctccttctactgcctggagtactttccttctcaaatgctgagaacgggcaacaacttccagtttacttacaccttcgaggacgtgcctttccacagcagctacgctcacagccagagtctggaccgtctcatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcctcacacaaagacgacaaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttaccttacccgtcccctgtaa

SEQ ID NO: 11 - DNA, 2208 bp, Artificial
  LP4-15 nucleotide sequence
atggctgccgatggttatcttccagattggctcgaggacaaccttagtgaaggaattcgcgagtggtgggctttgaaacctggagccccgaagcccaaagccaaccagcaaaagcaggacgacggccggggtctggtgcttcctggctacaagtacctcggacccttcaacggactcgacaaaggagagcccgtcaacgcggcggatgcagcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaacgcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccattgcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 12 - DNA, 2211 bp, Artificial
  LP4-19 nucleotide sequence
atggctgccgatggttatcttccagattggctcgaggacaaccttagtgaaggaattcgcgagtggtgggctttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcggacaataacgagggcgccgacggagtgggtaattcctcgggaaattggcattgcgattccacatggctgggcgacagagtcatcaccaccagcacccgaacctgggccctgcccacctacaacaaccacctctacaaacaaatttccagcgcttcaacgggagcctcgaacgacaatcactactttggctacagcaccccttgggggtattttgacttcaacagattccactgccacttttcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcggagtaccagttgccgtacgtcctcggctctgcgcaccagggctgcctccctccgttcccggcggacgtgttcatgattccgcagtacggctacctgacactcaacaatggcagccaggcagtgggacggtcatccttttactgcctggaatatttcccatcgcagatgctgagaaccggcaacaacttccagtttacttacaccttcaaggacgtgcctttccacagcagctacgcgcacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtttggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacttccaactactacaaatctacaagtgtggactttgctgttaatacagaaggcgtgtactctgaaccccgccccattggcacccgttacctcacccgtcccctgtaa

SEQ ID NO: 13 - DNA, 2211 bp, Artificial
  LP4-21 nucleotide sequence
atggctgccgatggttatcttcctgattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcggacaataacgagggcgccgacggagtgggtaattcctcgggaaattggcattgcgattccacatggctgggcgacagagtcatcaccaccagcacccgaacctgggccctgcccacttacaacaaccatctctacaagcaaatctccagtgcttcaacgggggccagcaacgacaaccactacttcggctacagcaccccctgggggtattttgacttcaacagattccactgccacttctcaccacgtgactggcagcgactcatcaacaacaactggggattccggcctaagcgactcaacttcaaactcttcaacattcaggtcaaagaggttacggacaacaatggagtcaagaccatcgccaataaccttaccagcacggtccaggtcttcacggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaataacaatagcaactttgcctggactgctgggaccaaataccatctgaatggaagaaattcattggctaatcctggcatcgctatggcctcacacaaagacgacaaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtaatctgtaa

SEQ ID NO: 14 - DNA, 2211 bp, Artificial
  LP4-24 nucleotide sequence
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctggctacaagtacctcggacccttcaacggactcgacaagggggagcccgtcaacgcggcggatgcagcggccctcgagcacgacaaggcctacgaccggcagctcaaagcgggtgacaatccgtacctgcggtataaccacgccgacgcggagtttcaggagcgccttaaagaagatacgtcttttgggggcaacctcgggcgagcagtcttccaggccaagaagagggttctcgaaccttttggtctggttgaggaaggcgctaagacggctcctggaaagaagaggcctgtagagcagtcgccacaagagccagactcctcctcgggcattggcaagacaggccagcagcccgctaaaaagagactcaattttggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggcgacagagtcatcaccaccagcacccgaacctgggccctgcccacttacaacaaccatctctacaagcaaatctccagtgcttcaacgggggccagcaacgacaaccactacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcctcacacaaagacgacaaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 15 - protein, 736 aa, Artificial
  LP1-1 amino acid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMASHKDDKERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 16 - protein, 736 aa, Artificial
  LP1-11 amino acid sequence
MAADGYLPDWLEDTLSEGIRQWWKLKPGPPPPKPAERHKDDSRGLVLPGYKYLGPFNGLDKGEPVNAADAAALEHDKAYDQQLQAGDNPYLRYNHADAEFQERLQEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMASHKDDKERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 17 - protein, 737 aa, Artificial
  LP1-13 amino acid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEQSPQEPDSSSGIGKTGQQPAKKRLNFGQTGDSESVPDPQPLGEPPAAPSGVGPNTMAAGGGAPMADNNEGADGVGSSSGNWHCDSQWLGDRVITTSTRTWTLPTYNNHLYKQISNSTSGGATNDNTYFGYSTPWGYFDFNRFHCHFSPRDWQRLINNNWGFRPKRLNFKLFNIQVKEVTDNNGVKTIANNLTSTVQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 18 - protein, 736 aa, Artificial
  LP1-18 amino acid sequence
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGSSSGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSEYQLPYVLGSAHQGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDKERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 19 - protein, 736 aa, Artificial
  LP1-23 amino acid sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRLLEPLGLVEEAAKTAPGKKRPVEQSPQEPDSSAGIGKSGAQPAKKRLNFGQTGGTESVPDPQPIGEPPAAPSGVGSLTMASGGGAPVADNNEGADGVGSSSGNWHCDSQWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMASHKDDKERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 20 - protein, 736 aa, Artificial
  LP2-10 amino acid sequence
MAADGYLPDWLEDTLSEGIRQWWKLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRLLEPLGLVEEAAKTAPGKKRPVEQSPQEPDSSAGIGKSGAQPAKKRLNFGQTGGTESVPDPQPLGEPPAAPSGVGPNTMAAGGGAPMADNNEGADGVGSSSGNWHCDSTWLGDRVITTSTRTWALPTYNNHLYKQISSASTGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDKERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 21 - protein, 736 aa, Artificial
  LP2-19 amino acid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEGAKTAPGKKRPVEQSPQEPDSSAGIGKSGAQPAKKRLNFGQTGDSESVPDPQPLGEPPAAPSGVGPNTMASGGGAPVADNNEGADGVGSSSGNWHCDSQWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYNLNGRNSLANPGIAMASHKDDKERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 22 - protein, 735 aa, Artificial
  LP2-20 amino acid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSTLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 23 - protein, 735 aa, Artificial
  LP3-19 amino acid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNSSGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTDNNGVKTIANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 24 - protein, 737 aa, Artificial
  LP4-7 amino acid sequence
MAADGYLPDWLEDNLSEGIREWWDLKPGAPKPKANQQKQDDGRGLVLPGYKYLGPFNGLDKGEPVNAADAAALEHDKAYDRQLDSGDNPYLKYNHADAEFQERLKEDTSFGGNLGRAVFQAKKRLLEPLGLVEEAAKTAPGKKRPVEQSPQEPDSSSGIGKTGQQPAKKRLNFGQTGDSESVPDPQPLGEPPATPAAVGPTTMASGGGAPMADNNEGADGVGSSSGNWHCDSQWLGDRVITTSTRTWALPTYNNHLYKQISNSTSGGATNDNTYFGYSTPWGYFDFNRFHCHFSPRDWQRLINNNWGFRPKRLNFKLFNIQVKEVTDNNGVKTIANNLTSTVQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMASHKDDKERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 25 - protein, 735 aa, Artificial
  LP4-15 amino acid sequence
MAADGYLPDWLEDNLSEGIREWWALKPGAPKPKANQQKQDDGRGLVLPGYKYLGPFNGLDKGEPVNAADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSTLQFSQAGASDIRDQSRNWLPGPCYRQQRVSKTSADNNNSEYSWTGATKYHLNGRDSLVNPGPAIASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 26 - protein, 736 aa, Artificial
  LP4-19 amino acid sequence
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNSSGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSEYQLPYVLGSAHQGCLPPFPADVFMIPQYGYLTLNNGSQAVGRSSFYCLEYFPSQMLRTGNNFQFTYTFKDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRPL

SEQ ID NO: 27 - protein, 736 aa, Artificial
  LP4-21 amino acid sequence
MAADGYLPDWLEDTLSEGIRQWWKLKPGPPPPKPAERHKDDSRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNSSGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMASHKDDKERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRNL

SEQ ID NO: 28 - protein, 736 aa, Artificial
  LP4-24 amino acid sequence
MAADGYLPDWLEDTLSEGIRQWWKLKPGPPPPKPAERHKDDSRGLVLPGYKYLGPFNGLDKGEPVNAADAAALEHDKAYDRQLKAGDNPYLRYNHADAEFQERLKEDTSFGGNLGRAVFQAKKRVLEPFGLVEEGAKTAPGKKRPVEQSPQEPDSSSGIGKTGQQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMASHKDDKERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 29 - protein, 14 aa, Artificial
  AAV1 VR1 loop
QISSASTGASNDNH

SEQ ID NO: 30 - protein, 14 aa, Artificial
  AAV2 VR1 loop
QISSQSGASNDNHY

SEQ ID NO: 31 - protein, 14 aa, Artificial
  AAV3b VR1 loop
QISSQSGASNDNHY

SEQ ID NO: 32 - protein, 11 aa, Artificial
  AAV4 VR1 loop
RLGESLQSNTY

SEQ ID NO: 33 - protein, 10 aa, Artificial
  AAV5 VR1 loop
SVDGSNANAY

SEQ ID NO: 34 - protein, 14 aa, Artificial
  AAV6 VR1 loop
QISSASTGASNDNH

SEQ ID NO: 35 - protein, 14 aa, Artificial
  AAV7 VR1 loop
QISSETAGSTNDNT

SEQ ID NO: 36 - protein, 15 aa, Artificial
  AAV8 VR1 loop
QISNGTSGGATNDNT

SEQ ID NO: 37 - protein, 15 aa, Artificial
  AAV9 VR1 loop
QISNSTSGGSSNDNA

SEQ ID NO: 38 - protein, 15 aa, Artificial
  AAV10 VR1 loop
QISNGTSGGSTNDNT

SEQ ID NO: 39 - protein, 12 aa, Artificial
  AAV11 VR1 loop
LRLGTTSSSNTY

SEQ ID NO: 40 - protein, 11 aa, Artificial
  AAV12 VR1 loop
LRIGTTANSNT

SEQ ID NO: 41 - protein, 14 aa, Artificial
  AAV13 VR1 loop
QISSQSGATNDNHY

(19) United States
(12) Patent Application Publication
(10) Pub. No.: US 2023/0304033 A1
(43) Pub. Date: Sep. 28, 2023

(54) MODIFIED ADENO-ASSOCIATED VIRUS VECTORS THAT EVADE NEUTRALIZING ANTIBODIES AND USES THEREOF

(71) Applicant: The University of North Carolina at Chapel Hill, Chapel Hill, NC (US)

(72) Inventors: Chengwen Li, Chapel Hill, NC (US); Richard Jude Samulski, Hillsborough, NC (US)

(21) Appl. No.: 17/997,791

(22) PCT Filed: May 3, 2021
(86) PCT No.: PCT/US2021/030395

§ 371 Date: Nov. 2, 2022

Related U.S. Application Data
(60) Provisional application No. 63/019,658, filed on May 4, 2020.

(51) Int. Cl. C12N 15/86; C07K 14/005; A61K 48/00

(52) U.S. Cl. CPC C12N 15/86; C07K 14/005; A61K 48/0041; C12N 2750/14143; C12N 2750/14122; C12N 2750/14171

(57) ABSTRACT

The present invention is in the field of recombinant adeno-associated virus (AAV) vectors. In particular, the invention relates to AAV vectors comprising modified AAV capsid proteins that evade neutralizing antibodies but are still able to transduce hepatocytes. The invention further relates to methods of using the AAV vectors to deliver products to subjects and treat diseases.

This application is a 35 U.S.C. § 371 national phase application of PCT Application PCT/US2021/030395 filed May 3, 2021, which claims the benefit, under 35 U.S.C. § 119(e), of U.S. Provisional Application No. 63/019,658, filed on May 4, 2020, the entire contents of each of which are incorporated by reference herein.

### GOVERNMENT SUPPORT

This invention was made with government support under grant numbers HL012549, HL112761, and AI117408 awarded by the National Institutes of Health.

The government has certain rights in the invention.

### FIELD OF THE INVENTION

The present invention is in the field of recombinant adeno-associated virus (AAV) vectors. In particular, the invention relates to AAV vectors comprising modified AAV capsid proteins that evade neutralizing antibodies but are still able to transduce hepatocytes. The invention further relates to methods of using the AAV vectors to deliver products to subjects and treat diseases.

### BACKGROUND OF THE INVENTION

Adeno-associated virus (AAV) vector is a popular and effective transgene delivery vehicle and has been successfully applied in clinical trials in many diseases such as Leber hereditary amaurosis (Cwerman-Thibault et al., C R Biol. 337:193 (2014)) and hemophilia B (Nathwani et al., New Engl. J. Med. 365:2357 (2011); Tuddenham, Haemophilia 18 Suppl 4:13 (2012); George et al., New Engl. J. Med. 377:2215 (2017)). AAV is a single-stranded DNA virus which is composed of the rep gene and the cap gene flanked by the inverted terminal repeats. AAV rep proteins are responsible for viral life cycle and help virus genome encapsidation. The AAV capsid gene encoding three capsid proteins which play a major role for virus tissue tropism. Replacement of the AAV rep and cap genes with a therapeutic transgene cassette in a virion form an AAV vector. Although therapeutic level of clotting factors has been achieved after liver targeting of AAV vectors encoding clotting factors VIII (FVIII) or IX (FIX) via systemic administration, two major concerns are raised: the transduction efficiency of AAV vectors in human hepatocytes and high prevalence of AAV neutralizing antibodies (Nabs). In pre-clinical trials for hemophilia therapy with AAV vectors, much more efficient hepatocyte transduction was observed. Usually approximately 100 fold higher FIX in mice or 10-fold higher FIX in primates was achieved when compared to patients in clinical trials using similar doses of AAV vector (Hurlbut et al., Mol. Ther. 18:1983 (2010)). These results indicate the AAV transduction efficiency in current animal models may not be predictive for clinical studies. It is imperative to develop an authentic animal model to examine AAV transduction efficiency in guiding future clinical trials. Recently, a chimeric mouse xenografted with human hepatocytes has been used to study AAV vector tropism in human hepatocytes (Vercauteren et al., Mol. Ther. 24:1042 (2016); Wang et al., Mol. Ther. 23:1877 (2015); Paulk et al., Mol. Ther. 26:289 (2018)). To explore novel AAV vectors for enhanced transduction, genetic modification of AAV capsid is popular using rational design or directed evolution. These approaches have been used to develop novel AAV mutants with high human hepatocyte tropism in chimeric mice (Paulk et al., Mol. Ther. 26:289 (2018)) #. To overcome AAV Nabs, several lab and clinical approaches have been investigated including coating the AAV virion surface to avoid Nab recognition (Lee et al., Biotechnol. Bioeng. 92:24 (2005)), deletion of Nabs by plasma-apheresis and elimination of B cells with antibodies (Meliani et al., Nat. Commun. 9:4098 (2018)), utilization of AAV empty virions as a decoy (Mingozzi et al., Science Transl. Med. 5:194ra192 (2013); Gao, et al. Mol. Ther. 1:20139 (2014)), and genetic modification of AAV capsids to modify epitopes recognized by Nabs (Tse et al., Proc. Natl. Acad. Sci. USA 114:E4812 (2017); Louis Jeune et al., Human Gene Ther. Meth. 24:59 (2013)). Engineering AAV capsids represent a very promising strategies to develop novel AAV vectors with the ability to evade Nabs. Similar to genetic modification of AAV capsid for transduction enhancement, approaches with rational design and directed evolution have also been used to exploit AAV variants for Nab evasion (Tse et al., Proc. Natl. Acad. Sci. USA 114:E4812 (2017)).

The present invention overcomes the mentioned short-comings of current AAV vectors by providing mutant AAV vectors that evade Nabs and exhibit human hepatocyte tropism.

### SUMMARY OF THE INVENTION

The present invention is based on part on the creation of modified AAV vectors with the ability to evade Nabs and retain human hepatocyte tropism. The strategy of directed evolution with an AAV shuffled library was used in chimeric mice xenografted with human hepatocytes in the presence of human Nabs. After 4 cycles of selection in mice in the presence of human IVIG, mutants were isolated composed of synthetic capsid proteins derived from portions of AAV2, 6, 8 and 9.

Nab analysis showed the mutant AAVs had at least some ability to escape Nab activity not only from IVIG but also from sera of healthy subjects or hemophilia patients when compared to wild-type AAV serotypes. The modified vectors also showed at least some maintenance of human hepatocyte tropism. The modified capsid proteins can be used advantageously for delivery and expression of proteins or functional nucleic acids in subjects, e.g., in the liver of subjects.

Thus, one aspect of the invention relates to a nucleic acid encoding an AAV capsid protein, the nucleic acid comprising an AAV capsid protein coding sequence that is at least 90% identical to:
(a) the nucleotide sequence of any one of SEQ ID NOS:1-14; or
(b) a nucleotide sequence encoding any one of SEQ ID NOS:15-28.

Another aspect of the invention relates to a nucleic acid encoding an AAV capsid protein, the nucleic acid comprising an AAV capsid protein coding sequence that encodes a capsid protein comprising one or more of the following mutations: a) Q105K;
b) A135G;
c) T179S;
d) I188L;
e) S200P;
f) L201N;
g) D348E;
h) E360Q;
i) D383N;
j) E417K;
k) R459T;
l) H510N;
m) M523I;
n) T526S;
o) P724H;
p) R725H;
q) N735P;
   based on the AAV8 capsid protein sequence or the corresponding residue in another AAV capsid protein.

An additional aspect of the invention relates to a nucleic acid encoding an AAV capsid protein, the nucleic acid comprising an AAV capsid protein coding sequence that encodes a capsid protein comprising one or more mutations in the variable region 1 (VR1) loop and or a chimeric capsid protein in which the VR1 loop has been replaced by a VR1 loop from a capsid protein of a different AAV serotype, wherein an AAV particle comprising the encoded capsid protein has decreased susceptibility to neutralizing antibodies when administered to a subject relative to an AAV particle comprising a wild-type capsid protein, and wherein the VR1 loop corresponds to amino acid residues QISNGTSGGATNDNT (SEQ ID NO:36) in the AAV8 capsid protein and the corresponding amino acids in other serotypes.

A further aspect of the invention relates to a vector, cell, or virus particle comprising the nucleic acid of the invention.

An additional aspect of the invention relates to an AAV capsid protein comprising an amino acid sequence at least 90% identical to any one of SEQ ID NOS:15-28.

Another aspect of the invention relates to a AAV capsid protein comprising one or more of the following mutations:
a) Q105K;
b) A135G;
c) T179S;
d) I188L;
e) S200P;
f) L201N;
g) D348E;
h) E360Q;
i) D383N;
j) E417K;
k) R459T;
l) H510N;
m) M523I;
n) T526S;
o) P724H;
p) R725H;
q) N735P;
   based on the AAV8 capsid protein sequence or the corresponding residue in another AAV capsid protein.

A further aspect of the invention relates to a modified AAV capsid protein comprises one or more mutations in the variable region 1 (VR1) loop and or a chimeric capsid protein in which the VR1 loop has been replaced by a VR1 loop from a capsid protein of a different AAV serotype, wherein an AAV particle comprising the encoded capsid protein has decreased susceptibility to neutralizing antibodies when administered to a subject relative to an AAV particle comprising a wild-type capsid protein, and wherein the VR1 loop corresponds to amino acid residues QISNGTSGGATNDNT (SEQ ID NO:36) in the AAV8 capsid protein and the corresponding amino acids in other serotypes.

Another aspect of the invention relates to an AAV particle comprising an AAV vector genome; and the modified capsid protein of the invention, wherein the AAV capsid encapsidates the AAV vector genome.

A further aspect of the invention relates to a method of producing a recombinant AAV particle comprising an AAV capsid, the method comprising providing a cell in vitro with a nucleic acid according to the invention, an AAV rep coding sequence, an AAV vector genome comprising a heterologous nucleic acid, and helper functions for generating a productive AAV infection; and allowing assembly of the recombinant AAV particle comprising the AAV capsid and encapsidating the AAV vector genome.

An additional aspect of the invention relates to a pharmaceutical formulation comprising the modified capsid protein, nucleic acid, virus particle, or AAV particle of the invention in a pharmaceutically acceptable carrier.

Another aspect of the invention relates to a method of delivering a nucleic acid of interest to a cell, e.g., a hepatocyte, the method comprising contacting the cell with the AAV particle of the invention.

A further aspect of the invention relates to a method of delivering a nucleic acid of interest to a cell, e.g., a hepatocyte, in a mammalian subject, the method comprising administering an effective amount of the AAV particle or pharmaceutical formulation of the invention to a mammalian subject.

Another aspect of the invention relates to a method of treating a disorder in a mammalian subject in need thereof, wherein the disorder is treatable by expressing a product in the subject, e.g., in the liver of the subject, the method comprising administering a therapeutically effective amount of the AAV particle or pharmaceutical formulation of the invention to a mammalian subject.

An additional aspect of the invention relates to a method of decreasing the susceptibility of an AAV particle to neutralizing antibodies when administered to a subject, comprising preparing the AAV particle with the capsid protein of the invention.

These and other aspects of the invention are set forth in more detail in the description of the invention below.

### BRIEF DESCRIPTION OF THE DRAWINGS

FIG. 1 shows a schematic diagram for isolation of AAV mutants from hepatocytes of humanized mice. Humanized mice were first pretreated by injecting 30 mg of IVIG via retro-orbital vein and then administered 2×10¹¹ particles of AAV shuffling library viruses. Three days later, adenovirus d1309 was injected into the mice, and at day 5, hepatocytes in the mice were harvested and lysed. The AAV in the cell lysate from humanized liver was re-injected into other IVIG pretreated mice for the next round of AAV amplification as described above. The AAV amplification was repeated two more times. After the last round of AAV amplification, DNA was extracted from humanized liver, and the capsid DNA was amplified using capsid specific primers and then reconstructed into the pXR backbone for sequencing and AAV vector production.

FIG. 2 shows a schematic diagram for capsids of AAV mutants isolated from the livers of humanized mice. Different patterns were used to indicate the composition of capsids from AAV serotypes and point mutations are shown on the top of each clone diagram. The frequency indicates the number of capsids isolated from 25 available clones.

FIGS. 3A-3B show AAV package capacity and transduction efficiency with mutants AAV capsids. (A) Package capacity of AAV mutants. The production efficiency of mutants and wild type AAV vectors was normalized to AAV2. (B) Transduction efficiency in Huh 7 cells from mutant AAV vectors. Huh7 was infected with 1×104 particles/cell of AAV vectors encoding the luciferase gene in 96 well plates. 48 hours later, the transduction efficiency was measured by luciferase assay.

FIG. 4 shows Nab analysis of AAV mutants against human IVIG. 1×10⁹ particles of AAV/luc vectors were pre-incubated with different concentrations of IVIG for 1 hr at 4° C., and then the mixtures were added into the cell culture medium of Huh7 cells in a 48 well-plate. Forty-eight hours later, the luciferase expression was measured and the Nab titer was determined.

FIGS. 5A-5B show the prevalence of neutralizing antibodies against AAV mutants in healthy subjects. (A) The AAV Nab prevalence in healthy human subjects. (B) The comparison of Nab titers for mutant AAV LP2-10 and serotypes in healthy individuals. * p<0.05when compared to other serotypes for Nab titer of mutant AAV LP2-10. NS: no significance.

FIGS. 6A-6B show the prevalence of neutralizing antibodies against AAV mutants in healthy subjects and hemophilia patients. (A) The AAV Nab prevalence in hemophilia patients. (B) The comparison of Nab titers for mutant AAV LP2-10 and serotypes in hemophilia patients. * p<0.05when compared to other serotypes for Nab titer of mutant AAV LP2-10. NS: no significance.

FIG. 7 shows the transduction of mutant AAV LP2-10 in human hepatocytes of chimeric mice. 1×10¹¹ particles of scAAV/GFP (AAV8 or mutant AAV LP2-10) were injected into human hepatocyte xenografted mice via retro-orbital vein, and three weeks later, the liver was harvested and analyzed by IHC. Antibody against human albumin was used to identify human hepatocytes (green), and GFP expression was stained in red.

FIGS. 8A-8C show the phylogeny of AAV mutants and structural model of mutant AAV LP2-10. (A) A phylogenetic tree was employed to analyze the genetic distance between wild type AAVs and Nab escape AAV mutants isolated from the livers of humanized mice. (B) The fragments and point mutations on LP2-10 capsid subunit was structurally analyzed. Capsid domains derived from different AAV serotypes in the VP3 subunit of mutant AAV LP2-10 and point mutations were highlighted as follows: white (AAV6), gray (AAV8), and dark gray (point mutations at residues 531, 725 and 735 position of AAV8 VP1). (C) The assembled full mutant AAV LP2-10 virion (60 mer) was displayed based on the subunit of VP3 in FIG. 8B.

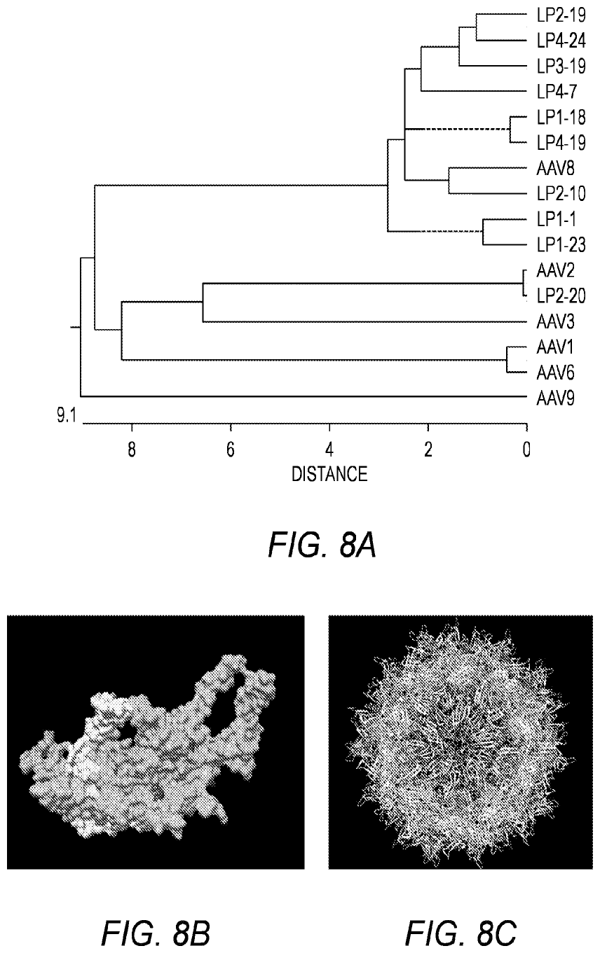

### DETAILED DESCRIPTION OF THE INVENTION

The present invention is based, in part, on the development of synthetic AAV capsid protein sequences that are capable of evading Nab while transducing hepatocytes in vivo and in vitro. The synthetic capsid proteins can be used to create AAV vectors for use in research or therapeutic applications, particularly where avoidance of Nab and vector inactivation is needed.

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for production of recombinant and synthetic polypeptides, antibodies or antigen-binding fragments thereof, manipulation of nucleic acid sequences, production of transformed cells, the construction of rAAV constructs, modified capsid proteins, packaging vectors expressing the AAV rep and/or cap sequences, and transiently and stably transfected packaging cells. Such techniques are known to those skilled in the art. See, e.g., SAMBROOK et al., MOLECULAR CLONING: A LABORATORY MANUAL 4th Ed. (Cold Spring Harbor, N Y, 2013); F. M. AUSUBEL et al. CURRENT PROTOCOLS IN MOLECULAR BIOLOGY (Green Publishing Associates, Inc. and John Wiley & Sons, Inc., New York).

All publications, patent applications, patents, nucleotide sequences, amino acid sequences and other references mentioned herein are incorporated by reference in their entirety.

### Definitions

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of+10%, ±5%, ±1%, +0.5%, or even ±0.1% of the specified amount.

The term “consisting essentially of” as used herein in connection with a nucleic acid, protein or capsid structure means that the nucleic acid, protein or capsid structure does not contain any element other than the recited element(s) that significantly alters (e.g., more than about 1%, 5% or 10%) the function of interest of the nucleic acid, protein or capsid structure, e.g., tropism profile of the protein or capsid or a protein or capsid encoded by the nucleic acid.

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence or between the two ends (e.g., between domains) such that the function of the polynucleotide or polypeptide is not materially altered. The total of ten or less additional nucleotides or amino acids includes the total number of additional nucleotides or amino acids added together.

The designation of all amino acid positions in the AAV capsid subunits in the description of the invention and the appended claims is with respect to VP1 capsid subunit numbering.

The term “adeno-associated virus” (AAV) in the context of the present invention includes without limitation AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV and any other AAV now known or later discovered. See, e.g., BERNARD N. FIELDS et al., VIROLOGY, volume 2, chapter 69 (4th ed., Lippincott-Raven Publishers). A number of additional AAV serotypes and clades have been identified (see, e.g., Gao et al., (2004) J. Virol. 78:6381-6388 and Table 1), which are also encompassed by the term “AAV.”

TABLE 1
AAV Genomes

| AAV | GenBank | AAV | GenBank | AAV | GenBank |
|---|---|---|---|---|---|
| Serotypes/ | Accession | Serotypes/ | Accession | Serotypes/ | Accession |
| Isolates | Number | Isolates | Number | Isolates | Number |
| Clonal Isolates | | Hu S17 | AY695376 | Cy3 | AY243019 |
| Avian AAV ATCC | AY186198, | Hu T88 | AY695375 | Cy5 | AY243017 |
| VR-865 | AY629583, | | | | |
| | NC_004828 | | | | |
| Avian AAV strain | NC_006263, | Hu T71 | AY695374 | Rh13 | AY243013 |
| DA-1 | AY629583 | | | | |
| Bovine AAV | NC_005889, | Hu T70 | AY695373 | | |
| | AY388617 | | | | |
| AAV4 | NC_001829 | Hu T40 | AY695372 | Clade E | |
| AAV5 | AY18065, | Hu T32 | AY695371 | Rh38 | AY530558 |
| | AF085716 | | | | |
| Rh34 | AY243001 | Hu T17 | AY695370 | Hu66 | AY530626 |
| Rh33 | AY243002 | Hu LG15 | AY695377 | Hu42 | AY530605 |
| Rh32 | AY243003 | | | Hu67 | AY530627 |
| AAV10 | AY631965 | Clade C | | Hu40 | AY530603 |
| AAV11 | AY631966 | AAV3 | NC_001729 | Hu41 | AY530604 |
| AAV12 | DQ813647 | AAV3B | NC_001863 | Hu37 | AY530600 |
| AAV13 | EU285562 | Hu9 | AY530629 | Rh40 | AY530559 |
| | | Hu10 | AY530576 | Rh2 | AY243007 |
| Clade A | | Hu11 | AY530577 | Bb1 | AY243023 |
| AAV1 | NC_002077 | Hu53 | AY530615 | Bb2 | AY243022 |
| | AF063497 | | | | |
| AAV6 | NC_001862 | Hu55 | AY530617 | Rh10 | AY243015 |
| Hu.48 | AY530611 | Hu54 | AY530616 | Hu17 | AY530582 |
| Hu 43 | AY530606 | Hu7 | AY530628 | Hu6 | AY530621 |
| Hu 44 | AY530607 | Hu18 | AY530583 | Rh25 | AY530557 |
| Hu 46 | AY530609 | Hu15 | AY530580 | Pi2 | AY530554 |
| | | Hu16 | AY530581 | Pi1 | AY530553 |
| Clade B | | Hu25 | AY530591 | Pi3 | AY530555 |
| Hu19 | AY530584 | Hu60 | AY530622 | Rh57 | AY530569 |
| Hu20 | AY530586 | Ch5 | AY243021 | Rh50 | AY530563 |
| Hu23 | AY530589 | Hu3 | AY530595 | Rh49 | AY530562 |
| Hu22 | AY530588 | Hu1 | AY530575 | Hu39 | AY530601 |
| Hu24 | AY530590 | Hu4 | AY530602 | Rh58 | AY530570 |
| Hu21 | AY530587 | Hu2 | AY530585 | Rh61 | AY530572 |
| Hu27 | AY530592 | Hu61 | AY530623 | Rh52 | AY530565 |
| Hu28 | AY530593 | | | Rh53 | AY530566 |
| Hu29 | AY530594 | Clade D | | Rh51 | AY530564 |
| Hu63 | AY530624 | Rh62 | AY530573 | Rh64 | AY530574 |
| Hu64 | AY530625 | Rh48 | AY530561 | Rh43 | AY530560 |
| Hu13 | AY530578 | Rh54 | AY530567 | AAV8 | AF513852 |
| Hu56 | AY530618 | Rh55 | AY530568 | Rh8 | AY242997 |
| Hu57 | AY530619 | Cy2 | AY243020 | Rh1 | AY530556 |
| Hu49 | AY530612 | AAV7 | AF513851 | | |
| Hu58 | AY530620 | Rh35 | AY243000 | Clade F | |
| Hu34 | AY530598 | Rh37 | AY242998 | AAV9 (Hu14) | AY530579 |
| Hu35 | AY530599 | Rh36 | AY242999 | Hu31 | AY530596 |
| AAV2 | NC_001401 | Cy6 | AY243016 | Hu32 | AY530597 |
| Hu45 | AY530608 | Cy4 | AY243018 | | |
| Hu47 | AY530610 | | | | |
| Hu51 | AY530613 | | | | |
| Hu52 | AY530614 | | | | |
| Hu T41 | AY695378 | | | | |

The genomic sequences of various AAV and autonomous parvoviruses, as well as the sequences of the ITRs, Rep proteins, and capsid subunits are known in the art. Such sequences may be found in the literature or in public databases such as the GenBank® database. See, e.g., GenBank® Accession Numbers NC 002077, NC 001401, NC 001729, NC 001863, NC 001829, NC 001862, NC 000883, NC 001701, NC 001510, AF063497, U89790, AF043303, AF028705, AF028704, J02275, J01901, J02275, X01457, AF288061, AH009962, AY028226, AY028223, NC 001358, NC 001540, AF513851, AF513852, AY530579, AY631965, AY631966; the disclosures of which are incorporated herein in their entirety. See also, e.g., Srivistava et al., (1983) J. Virol. 45:555; Chiorini et al., (1998) J. Virol. 71:6823; Chiorini et al., (1999) J. Virol. 73:1309; Bantel-Schaal et al., (1999) J. Virol. 73:939; Xiao et al., (1999) J. Virol. 73:3994; Muramatsu et al., (1996) Virology 221:208; Shade et al., (1986) J. Virol. 58:921; Gao et al., (2002) Proc. Nat. Acad. Sci. USA 99:11854; international patent publications WO 00/28061, WO 99/61601, WO 98/11244; U.S. Pat. No. 6,156,303; the disclosures of which are incorporated herein in their entirety. See also Table 1. An early description of the AAV1, AAV2 and AAV3 terminal repeat sequences is provided by Xiao, X., (1996), “Characterization of Adeno-associated virus (AAV) DNA replication and integration,” Ph.D. Dissertation, University of Pittsburgh, Pittsburgh, PA (incorporated herein it its entirety).

A “chimeric” AAV nucleic acid capsid coding sequence or AAV capsid protein is one that combines portions of two or more capsid sequences. A “chimeric” AAV virion or particle comprises a chimeric AAV capsid protein.

The term “tropism” as used herein refers to preferential entry of the virus into certain cell or tissue type(s) and/or preferential interaction with the cell surface that facilitates entry into certain cell or tissue types, optionally and preferably followed by expression (e.g., transcription and, optionally, translation) of sequences carried by the viral genome in the cell, e.g., for a recombinant virus, expression of the heterologous nucleotide sequence(s). Those skilled in the art will appreciate that transcription of a heterologous nucleic acid sequence from the viral genome may not be initiated in the absence of trans-acting factors, e.g., for an inducible promoter or otherwise regulated nucleic acid sequence. In the case of a rAAV genome, gene expression from the viral genome may be from a stably integrated provirus and/or from a non-integrated episome, as well as any other form which the virus nucleic acid may take within the cell.

The term “tropism profile” refers to the pattern of transduction of one or more target cells, tissues and/or organs. Representative examples of synthetic AAV capsids have a tropism profile characterized by efficient transduction of cells of the liver with only low transduction of other organs.

The term “specific for hepatocytes” as used herein refers to a viral vector that, when administered in vivo, preferentially transduces hepatocytes with minimal transduction of cells outside the liver. In some embodiments, at least about 80% of the transduced cells are hepatocytes, e.g., at least about 85%, 90%, 95%, 96%, 97%, 98%, 99% or more hepatocytes.

The term “disorder is treatable by expressing a product in the subject” or “in the liver” as used herein refers to a disease, disorder, or injury in which expression of a product (e.g., a protein or polynucleotide) in the subject, e.g., in the liver, provides an effective treatment or prevention of the disorder.

As used herein, “transduction” of a cell by a virus vector (e.g., an AAV vector) means entry of the vector into the cell and transfer of genetic material into the cell by the incorporation of nucleic acid into the virus vector and subsequent transfer into the cell via the virus vector.

Unless indicated otherwise, “efficient transduction” or “efficient tropism,” or similar terms, can be determined by reference to a suitable positive or negative control (e.g., at least about 50%, 60%, 70%, 80%, 85%, 90%, 95% or more of the transduction or tropism, respectively, of a positive control or at least about 110%, 120%, 150%, 200%, 300%, 500%, 1000% or more of the transduction or tropism, respectively, of a negative control).

Similarly, it can be determined if a virus “does not efficiently transduce” or “does not have efficient tropism” for a target tissue, or similar terms, by reference to a suitable control. In particular embodiments, the virus vector does not efficiently transduce (i.e., does not have efficient tropism for) tissues outside the liver, e.g., muscle, kidney, gonads and/or germ cells. In particular embodiments, undesirable transduction of tissue(s) (e.g., liver) is 20% or less, 10% or less, 5% or less, 1% or less, 0.1% or less of the level of transduction of the desired target tissue(s) (e.g., hepatocytes).

As used herein, the term “polypeptide” encompasses both peptides and proteins, unless indicated otherwise.

A “nucleic acid” or “nucleotide sequence” is a sequence of nucleotide bases, and may be RNA, DNA or DNA-RNA hybrid sequences (including both naturally occurring and non-naturally occurring nucleotide), but is preferably either single or double stranded DNA sequences.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

By the term “treat,” “treating,” or “treatment of” (or grammatically equivalent terms) it is meant that the severity of the subject's condition is reduced or at least partially improved or ameliorated and/or that some alleviation, mitigation or decrease in at least one clinical symptom is achieved and/or there is a delay in the progression of the condition and/or prevention or delay of the onset of a disease or disorder.

As used herein, the term “prevent,” “prevents,” or “prevention” (and grammatical equivalents thereof) refers to a delay in the onset of a disease or disorder or the lessening of symptoms upon onset of the disease or disorder. The terms are not meant to imply complete abolition of disease and encompasses any type of prophylactic treatment that reduces the incidence of the condition or delays the onset and/or progression of the condition.

An “effective” or “therapeutically effective” amount as used herein is an amount that is sufficient to provide some improvement or benefit to the subject. Alternatively stated, an “effective” or “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject.

A “heterologous nucleotide sequence” or “heterologous nucleic acid” is a sequence that is not naturally occurring in the virus. Generally, the heterologous nucleic acid or nucleotide sequence comprises an open reading frame that encodes a polypeptide and/or a nontranslated RNA.

A “therapeutic polypeptide” can be a polypeptide that can alleviate or reduce symptoms that result from an absence or defect in a protein in a cell or subject. In addition, a “therapeutic polypeptide” can be a polypeptide that otherwise confers a benefit to a subject, e.g., anti-cancer effects or improvement in transplant survivability.

As used herein, the term “vector,” “virus vector,” “delivery vector” (and similar terms) generally refers to a virus particle that functions as a nucleic acid delivery vehicle, and which comprises the viral nucleic acid (i.e., the vector genome) packaged within the virion. Virus vectors according to the present invention comprise a synthetic AAV capsid according to the invention and can package an AAV or rAAV genome or any other nucleic acid including viral nucleic acids. Alternatively, in some contexts, the term “vector,” “virus vector,” “delivery vector” (and similar terms) may be used to refer to the vector genome (e.g., vDNA) in the absence of the virion and/or to a viral capsid that acts as a transporter to deliver molecules tethered to the capsid or packaged within the capsid.

A “recombinant AAV vector genome” or “rAAV genome” is an AAV genome (i.e., vDNA) that comprises at least one inverted terminal repeat (e.g., one, two or three inverted terminal repeats) and one or more heterologous nucleotide sequences. rAAV vectors generally retain the 145 base terminal repeat(s) (TR(s)) in cis to generate virus; however, modified AAV TRs and non-AAV TRs including partially or completely synthetic sequences can also serve this purpose. All other viral sequences are dispensable and may be supplied in trans (Muzyczka, (1992) Curr. Topics Microbiol. Immunol. 158:97). The rAAV vector optionally comprises two TRs (e.g., AAV TRs), which generally will be at the 5′ and 3′ ends of the heterologous nucleotide sequence(s), but need not be contiguous thereto. The TRs can be the same or different from each other. The vector genome can also contain a single ITR at its 3′ or 5′ end.

The term “terminal repeat” or “TR” includes any viral terminal repeat or synthetic sequence that forms a hairpin structure and functions as a terminal repeat (i.e., mediates the desired functions such as replication, virus packaging, integration and/or provirus rescue, and the like). The TR can be an AAV TR or a non-AAV TR.

For example, a non-AAV TR sequence such as those of other parvoviruses (e.g., canine parvovirus (CPV), mouse parvovirus (MVM), human parvovirus B-19) or the SV40 hairpin that serves as the origin of SV40 replication can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

An “AAV inverted terminal repeat” or “AAV ITR” may be from any AAV, including but not limited to serotypes 1, 2, 3, 4, 5, 6, 7, 8, 9, 10, 11, 12, or 13 or any other AAV now known or later discovered (see, e.g., Table 1). An AAV ITR need not have the native ITR sequence (e.g., a native AAV ITR sequence may be altered by insertion, deletion, truncation and/or missense mutations), as long as the terminal repeat mediates the desired functions, e.g., replication, virus packaging, integration, and/or provirus rescue, and the like.

The terms “rAAV particle” and “rAAV virion” are used interchangeably here. A “rAAV particle” or “rAAV virion” comprises a rAAV vector genome packaged within an AAV capsid.

The AAV capsid structure is described in more detail in BERNARD N. FIELDS et al., VIROLOGY, volume 2, chapters 69 & 70 (4th ed., Lippincott-Raven Publishers).

By “substantially retain” a property, it is meant that at least about 75%, 85%, 90%, 95%, 97%, 98%, 99% or 100% of the property (e.g., activity or other measurable characteristic) is retained.

### Modified Capsid Proteins

The inventors have developed modified capsid proteins capable of evading Nab while providing transduction of human and other mammalian hepatocytes in vivo and in vitro. Thus, one aspect of the invention relates to an AAV capsid protein comprising, consisting essentially of, or consisting of an amino acid sequence at least 90% identical to any one of SEQ ID NOS:15-28. In some embodiments, the modified capsid protein comprises, consists essentially of, or consists of an amino acid sequence at least 91%, 92%, 93%, 94%, 95%, 96%, 97%, 98%, 99%, 99.5% identical to the amino acid sequence of any one of SEQ ID NOS:15-28. In some embodiments, the modified capsid protein comprises, consists essentially of, or consists of the amino acid sequence of any one of SEQ ID NOS:15-28. In some embodiments, the modified capsid protein comprises, consists essentially of, or consists of an amino acid sequence at least 91%, 92%, 93%, 94%, 95%, 96%, 97%, 98%, 99%, 99.5% identical to the amino acid sequence of any one of SEQ ID NO:20. In some embodiments, the modified capsid protein comprises, consists essentially of, or consists of the amino acid sequence of any one of SEQ ID NO:20.

Another aspect of the invention relates to a modified capsid protein comprising, consisting essentially of, or consisting of one or more of the following mutations:
a) Q105K;
b) A135G;
c) T179S;
d) I188L;
e) S200P;
f) L201N;
g) D348E;
h) E360Q;
i) D383N;
j) E417K;
k) R459T;
l) H510N;
m) M523I;
n) T526S;
o) P724H;
p) R725H;
q) N735P;
   based on the AAV8 capsid protein sequence or the corresponding residue in another AAV capsid protein. In some embodiments, the capsid protein, comprises 2, 3, 4, 5, or 6 or more of the listed mutations.

In some embodiments, the modified capsid protein further comprises an E531K mutation based on the AAV8 capsid protein sequence or the corresponding residue in another AAV capsid protein.

In some embodiments, the capsid protein may comprise one of the following combinations of mutations:
a) T526S, E531K, R725H, N735P;
b) T526S, E531K;
c) R725H, N735P;
d) D348E, E360Q, E531K;
e) D383N, T526S, E531K, R725H, N735P;
f) E531K, R725H, N735P;
g) A135G, T179S, I188L, S200P, L201N, D348E, E360Q, D383N, H51ON, E531K, P724H, N735P;
h) R459T;
i) E532K; R725H, N735P;
j) R459T, M523I;
k) E417K, N735P;
l) E531K, R725H; or
m) Q105K, T526S, E531K.

In some embodiments, the modified AAV capsid protein comprises one or more mutations in the variable region 1 (VR1) loop and or a chimeric capsid protein in which the VR1 loop has been replaced by a VR1 loop from a capsid protein of a different AAV serotype, wherein an AAV particle comprising the encoded capsid protein has decreased susceptibility to neutralizing antibodies when administered to a subject relative to an AAV particle comprising a wild-type capsid protein, and wherein the VR1 loop corresponds to amino acid residues QISNGTSGGATNDNT (SEQ ID NO:36) in the AAV8 capsid protein and the corresponding amino acids in other serotypes. VR1 loop sequences for several AAV serotypes are shown in Table 2. While specific residues are listed, it will be understood by one of skill in the art that the boundaries of the VR1 region are approximate and may vary by one or two residues on either end of the listed sequence.

TABLE 2

| Serotype | VR1 sequence | SEQ ID NO |
|---|---|---|
| AAV1 | QISSASTGASNDNH | 29 |
| AAV2 | QISSQSGASNDNHY | 30 |
| AAV3B | QISSQSGASNDNHY | 31 |
| AAV4 | RLGESLQSNTY | 32 |
| AAV5 | SVDGSNANAY | 33 |
| AAV6 | QISSASTGASNDNH | 34 |
| AAV7 | QISSETAGSTNDNT | 35 |
| AAV8 | QISNGTSGGATNDNT | 36 |
| AAV9 | QISNSTSGGSSNDNA | 37 |
| AAV10 | QISNGTSGGSTNDNT | 38 |
| AAV11 | LRLGTTSSSNTY | 39 |
| AAV12 | LRIGTTANSNT | 40 |
| AAV13 | QISSQSGATNDNHY | 41 |

In some embodiments, the modified capsid protein comprises most or all of the VP3 sequence of AAV8 at the C-terminus, e.g., 80%, 85%, 90%, 95%, or more of the AAV8 VP3 sequence counting from the C-terminus. In some embodiments, the AAV8 VP3 sequence in the modified capsid protein contains one or more of the mutations or combinations of mutations listed above. In some embodiments, the AAV8 VP3 sequence contains the mutations E532K; R725H, and N735P.

In some embodiments, the modified capsid protein comprises, consists essentially of, or consists of an amino acid sequence as shown in any one of SEQ ID NOS:15-28, wherein 1, 2 or fewer, 3 or fewer, 4 or fewer, 5 or fewer, 6 or fewer, 7 or fewer, 8 or fewer, 9 or fewer, 10 or fewer, 12 or fewer, 15 or fewer, 20 or fewer, 25 or fewer, 30 or fewer, 40 or fewer, or 50 or fewer of the amino acids is substituted by another amino acid (naturally occurring, modified and/or synthetic), optionally a conservative amino acid substitution, and/or 1, 2 or fewer, 3 or fewer, 4 or fewer, 5 or fewer, 6 or fewer, 7 or fewer, 8 or fewer, 9 or fewer, 10 or fewer, 12 or fewer, 15 or fewer, 20 or fewer, 25 or fewer, 30 or fewer, 40 or fewer, or 50 or fewer of the amino acids is deleted and/or there are insertions (including N-terminal and C-terminal extensions) of 1, 2 or fewer, 3 or fewer, 4 or fewer, 5 or fewer, 6 or fewer, 7 or fewer, 8 or fewer, 9 or fewer, 10 or fewer, 12 or fewer, 15 or fewer, 20 or fewer, 25 or fewer, 30 or fewer, 40 or fewer, or 50 or fewer amino acids or any combination of substitutions, deletions and/or insertions, wherein the substitutions, deletions and/or insertions do not unduly impair the structure and/or function of a virion (e.g., an AAV virion) comprising the variant capsid protein or capsid. For example, in representative embodiments of the invention, an AAV virion comprising the modified capsid protein substantially retains at least one property of a synthetic virion comprising a modified capsid protein as shown in one of SEQ ID NOS:15-28. For example, the virion comprising the modified capsid protein can substantially retain the Nab evasion and/or liver tropism profile of a virion comprising the modified AAV capsid protein as shown in SEQ ID NOS:15-28. Methods of evaluating biological properties such as virus transduction are well-known in the art (see, e.g., the Examples).

The designation of all amino acid positions in the description of the invention and the appended claims is with respect to VP1 numbering. Those skilled in the art will understand that the AAV capsid generally contains the smaller VP2 and VP3 capsid proteins as well. Due to the overlap of the coding sequences for the AAV capsid proteins, the nucleic acid coding sequences and amino acid sequences of the VP2 and VP3 capsid proteins will be apparent from the VP1 sequences shown in the disclosed sequences. In certain embodiments, isolated VP2 and VP3 capsid proteins comprising the sequences of the invention and isolated nucleic acids encoding the VP2 or VP3 proteins, or both, are contemplated. Also contemplated are chimeric capsid proteins comprising the VP2 or VP3 sequences of the invention.

Conservative amino acid substitutions are known in the art. In particular embodiments, a conservative amino acid substitution includes substitutions within one or more of the following groups: glycine, alanine; valine, isoleucine, leucine; aspartic acid, glutamic acid; asparagine, glutamine; serine, threonine; lysine, arginine; and/or phenylalanine, tyrosine.

It will be apparent to those skilled in the art that the amino acid sequences of the modified AAV capsid protein of SEQ ID NOS:15-28 can further be modified to incorporate other modifications as known in the art to impart desired properties. As nonlimiting possibilities, the capsid protein can be modified to incorporate targeting sequences (e.g., RGD) or sequences that facilitate purification and/or detection. For example, the capsid protein can be fused to all or a portion of glutathione-S-transferase, maltose-binding protein, a heparin/heparan sulfate binding domain, poly-His, a ligand, and/or a reporter protein (e.g., Green Fluorescent Protein, β-glucuronidase, β-galactosidase, luciferase, etc.), an immunoglobulin Fc fragment, a single-chain antibody, hemagglutinin, c-myc, FLAG epitope, and the like to form a fusion protein. Methods of inserting targeting peptides into the AAV capsid are known in the art (see, e.g., international patent publication WO 00/28004; Nicklin et al., (2001) Mol. Ther. 474-181; White et al., (2004) Circulation 109:513-319; Muller et al., (2003) Nature Biotech. 21:1040-1046.

The invention also provides AAV capsids comprising the modified capsid proteins of the invention and virus particles (i.e., virions) comprising the same, wherein the virus particle packages (i.e., encapsidates) a vector genome, optionally an AAV vector genome. In particular embodiments, the invention provides an AAV particle comprising an AAV capsid comprising an AAV capsid protein of the invention, wherein the AAV capsid packages an AAV vector genome. The invention also provides an AAV particle comprising an AAV capsid or AAV capsid protein encoded by the synthetic nucleic acid capsid coding sequences of the invention.

The viruses of the invention can further comprise a duplexed viral genome as described in international patent publication WO 01/92551 and U.S. Pat. No. 7,465,583.

In particular embodiments, the virion is a recombinant vector comprising a heterologous nucleic acid of interest, e.g., for delivery to a cell. Thus, the present invention is useful for the delivery of nucleic acids to cells in vitro, ex vivo, and in vivo. In representative embodiments, the recombinant vector of the invention can be advantageously employed to deliver or transfer nucleic acids to animal (e.g., mammalian) cells.

Any heterologous nucleotide sequence(s) may be delivered by a virus vector of the present invention. Nucleic acids of interest include nucleic acids encoding polypeptides, optionally therapeutic (e.g., for medical or veterinary uses) and/or immunogenic (e.g., for vaccines) polypeptides.

Therapeutic polypeptides include, but are not limited to, cystic fibrosis transmembrane regulator protein (CFTR), dystrophin (including the protein product of dystrophin mini-genes or micro-genes, see, e.g., Vincent et al., (1993) Nature Genetics 5:130; U.S. Patent Publication No. 2003017131; Wang et al., (2000) Proc. Natl. Acad. Sci. USA 97:13714-9 [mini-dystrophin]; Harper et al., (2002) Nature Med. 8:253-61 [micro-dystrophin]); mini-agrin, a laminin-α2, a sarcoglycan (α, β, γ or δ), Fukutin-related protein, myostatin pro-peptide, follistatin, dominant negative myostatin, an angiogenic factor (e.g., VEGF, angiopoietin-1 or 2), an anti-apoptotic factor (e.g., heme-oxygenase-1, TGF-β, inhibitors of pro-apoptotic signals such as caspases, proteases, kinases, death receptors [e.g., CD-095], modulators of cytochrome C release, inhibitors of mitochondrial pore opening and swelling); activin type II soluble receptor, anti-inflammatory polypeptides such as the Ikappa B dominant mutant, sarcospan, utrophin, mini-utrophin, antibodies or antibody fragments against myostatin or myostatin propeptide, cell cycle modulators, Rho kinase modulators such as Cethrin, which is a modified bacterial C3 exoenzyme [available from BioAxone Therapeutics, Inc., Saint-Lauren, Quebec, Canada], BCL-xL, BCL2, XIAP, FLICEc-s, dominant-negative caspase-8, dominant negative caspase-9, SPI-6 (see, e.g., U.S. Patent Application No. 20070026076), transcriptional factor PGC-α1, Pinch gene, ILK gene and thymosin P4 gene), clotting factors (e.g., Factor VIII, Factor IX, Factor X, etc.), erythropoietin, angiostatin, endostatin, catalase, tyrosine hydroxylase, an intracellular and/or extracellular superoxide dismutase, leptin, the LDL receptor, neprilysin, lipoprotein lipase, ornithine transcarbamylase, β-globin, α-globin, spectrin, α₁-antitrypsin, methyl cytosine binding protein 2, adenosine deaminase, hypoxanthine guanine phosphoribosyl transferase, β-glucocerebrosidase, sphingomyelinase, lysosomal hexosaminidase A, branched-chain keto acid dehydrogenase, RP65 protein, a cytokine (e.g., α-interferon, β-interferon, interferon-γ, interleukins-1 through −14, granulocyte-macrophage colony stimulating factor, lymphotoxin, and the like), peptide growth factors, neurotrophic factors and hormones (e.g., somatotropin, insulin, insulin-like growth factors including IGF-1 and IGF-2, GLβ-1, platelet derived growth factor, epidermal growth factor, fibroblast growth factor, nerve growth factor, neurotrophic factor −3 and −4, brain-derived neurotrophic factor, glial derived growth factor, transforming growth factor −α and −P, and the like), bone morphogenic proteins (including RANKL and VEGF), a lysosomal protein, a glutamate receptor, a lymphokine, soluble CD4, an Fc receptor, a T cell receptor, ApoE, ApoC, inhibitor 1 of protein phosphatase inhibitor 1 (I-1), phospholamban, serca2a, lysosomal acid α-glucosidase, α-galactosidase A, Barkct, β2-adrenergic receptor, β2-adrenergic receptor kinase (BARK), phosphoinositide-3 kinase (PI3 kinase), calsarcin, a receptor (e.g., the tumor necrosis growth factor-α soluble receptor), an anti-inflammatory factor such as IRAP, Pim-1, PGC-1α, SOD-1, SOD-2, ECF-SOD, kallikrein, thymosin-04, hypoxia-inducible transcription factor [HIF], an angiogenic factor, S100A1, parvalbumin, adenylyl cyclase type 6, a molecule that effects G-protein coupled receptor kinase type 2 knockdown such as a truncated constitutively active bARKct; phospholamban inhibitory or dominant-negative molecules such as phospholamban S16E, a monoclonal antibody (including single chain monoclonal antibodies) or a suicide gene product (e.g., thymidine kinase, cytosine deaminase, diphtheria toxin, and tumor necrosis factors such as TNF-α), and any other polypeptide that has a therapeutic effect in a subject in need thereof.

Heterologous nucleotide sequences encoding polypeptides include those encoding reporter polypeptides (e.g., an enzyme). Reporter polypeptides are known in the art and include, but are not limited to, a fluorescent protein (e.g., EGFP, GFP, RFP, BFP, YFP, or dsRED2), an enzyme that produces a detectable product, such as luciferase (e.g., from Gaussia, Renilla, or Photinus), β-galactosidase, β-glucuronidase, alkaline phosphatase, and chloramphenicol acetyltransferase gene, or proteins that can be directly detected. Virtually any protein can be directly detected by using, for example, specific antibodies to the protein. Additional markers (and associated antibiotics) that are suitable for either positive or negative selection of eukaryotic cells are disclosed in Sambrook et al., Molecular Cloning, A Laboratory Manual (4th Ed. 2013) (Cold Spring Harbor Laboratory), and Ausubel et al. (1992), Current Protocols in Molecular Biology, John Wiley & Sons, including periodic updates.

Alternatively, the heterologous nucleic acid may encode a functional RNA, e.g., an antisense oligonucleotide, a ribozyme (e.g., as described in U.S. Pat. No. 5,877,022), RNAs that effect spliceosome-mediated trans-splicing (see, Puttaraju et al., (1999) Nature Biotech. 17:246; U.S. Pat. Nos. 6,013,487and 6,083,702), interfering RNAs (RNAi) including small interfering RNAs (siRNA) that mediate gene silencing (see, Sharp et al., (2000) Science 287:2431), microRNA, or other non-translated “functional” RNAs, such as “guide” RNAs (Gorman et al., (1998) Proc. Nat. Acad. Sci. USA 95:4929; U.S. Pat. No. 5,869,248 to Yuan et al.), and the like. Exemplary untranslated RNAs include RNAi or antisense RNA against the multiple drug resistance (MDR) gene product (e.g., to treat tumors and/or for administration to the heart to prevent damage by chemotherapy), RNAi or antisense RNA against myostatin (Duchenne or Becker muscular dystrophy), RNAi or antisense RNA against VEGF or a tumor immunogen including but not limited to those tumor immunogens specifically described herein (to treat tumors), RNAi or antisense oligonucleotides targeting mutated dystrophins (Duchenne or Becker muscular dystrophy), RNAi or antisense RNA against the hepatitis B surface antigen gene (to prevent and/or treat hepatitis B infection), RNAi or antisense RNA against the HIV tat and/or rev genes (to prevent and/or treat HIV) and/or RNAi or antisense RNA against any other immunogen from a pathogen (to protect a subject from the pathogen) or a defective gene product (to prevent or treat disease). RNAi or antisense RNA against the targets described above or any other target can also be employed as a research reagent.

As is known in the art, anti-sense nucleic acids (e.g., DNA or RNA) and inhibitory RNA (e.g., microRNA and RNAi such as siRNA or shRNA) sequences can be used to induce “exon skipping” in patients with muscular dystrophy arising from defects in the dystrophin gene. Thus, the heterologous nucleic acid can encode an antisense nucleic acid or inhibitory RNA that induces appropriate exon skipping. Those skilled in the art will appreciate that the particular approach to exon skipping depends upon the nature of the underlying defect in the dystrophin gene, and numerous such strategies are known in the art. Exemplary antisense nucleic acids and inhibitory RNA sequences target the upstream branch point and/or downstream donor splice site and/or internal splicing enhancer sequence of one or more of the dystrophin exons (e.g., exons 19 or 23). For example, in particular embodiments, the heterologous nucleic acid encodes an antisense nucleic acid or inhibitory RNA directed against the upstream branch point and downstream splice donor site of exon 19 or 23 of the dystrophin gene. Such sequences can be incorporated into an AAV vector delivering a modified U7 snRNA and the antisense nucleic acid or inhibitory RNA (see, e.g., Goyenvalle et al., (2004) Science 306:1796-1799). As another strategy, a modified U1 snRNA can be incorporated into an AAV vector along with siRNA, microRNA or antisense RNA complementary to the upstream and downstream splice sites of a dystrophin exon (e.g., exon 19 or 23) (see, e.g., Denti et al., (2006) Proc. Nat. Acad. Sci. USA 103:3758-3763). Further, antisense nucleic acids and inhibitory RNA can target the splicing enhancer sequences within exons 19, 43, 45 or 53 (see, e.g., U.S. Pat. Nos. 6,653,467; 6,727,355; and 6,653,466).

The recombinant virus vector may also comprise a heterologous nucleotide sequence that shares homology with and recombines with a locus on the host chromosome. This approach may be utilized to correct a genetic defect in the host cell.

The present invention also provides recombinant virus vectors that express an immunogenic polypeptide, e.g., for vaccination. The heterologous nucleic acid may encode any immunogen of interest known in the art including, but are not limited to, immunogens from human immunodeficiency virus, influenza virus, gag proteins, tumor antigens, cancer antigens, bacterial antigens, viral antigens, and the like. Alternatively, the immunogen can be presented in the virus capsid (e.g., incorporated therein) or tethered to the virus capsid (e.g., by covalent modification).

The use of parvoviruses as vaccines is known in the art (see, e.g., Miyamura et al., (1994) Proc. Nat. Acad. Sci. USA 91:8507; U.S. Pat. No. 5,916,563 to Young et al., U.S. Pat. No. 5,905,040 to Mazzara et al., U.S. Pat. Nos. 5,882,652and 5,863,541 to Samulski et al.; the disclosures of which are incorporated herein in their entireties by reference). The antigen may be presented in the virus capsid. Alternatively, the antigen may be expressed from a heterologous nucleic acid introduced into a recombinant vector genome.

An immunogenic polypeptide, or immunogen, may be any polypeptide suitable for protecting the subject against a disease, including but not limited to microbial, bacterial, protozoal, parasitic, fungal and viral diseases. For example, the immunogen may be an orthomyxovirus immunogen (e.g., an influenza virus immunogen, such as the influenza virus hemagglutinin (HA) surface protein or the influenza virus nucleoprotein gene, or an equine influenza virus immunogen), or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SIV matrix/capsid proteins, and the HIV or SIV gag, pol and env genes products). The immunogen may also be an arenavirus immunogen (e.g., Lassa fever virus immunogen, such as the Lassa fever virus nucleocapsid protein gene and the Lassa fever envelope glycoprotein gene), a poxvirus immunogen (e.g., vaccinia, such as the vaccinia L1 or L8 genes), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP genes), a bunyavirus immunogen (e.g., RVFV, CCHF, and SFS viruses), or a coronavirus immunogen (e.g., an infectious human coronavirus immunogen, such as the human coronavirus envelope glycoprotein gene, or a porcine transmissible gastroenteritis virus immunogen, or an avian infectious bronchitis virus immunogen, or a severe acute respiratory syndrome (SARS) immunogen such as a S [S1 or S2], M, E, or N protein or an immunogenic fragment thereof). The immunogen may further be a polio immunogen, herpes immunogen (e.g., CMV, EBV, HSV immunogens) mumps immunogen, measles immunogen, rubella immunogen, diphtheria toxin or other diphtheria immunogen, pertussis antigen, hepatitis (e.g., hepatitis A, hepatitis B or hepatitis C) immunogen, or any other vaccine immunogen known in the art.

Alternatively, the immunogen may be any tumor or cancer cell antigen. Optionally, the tumor or cancer antigen is expressed on the surface of the cancer cell. Exemplary cancer and tumor cell antigens are described in S. A. Rosenberg, (1999) Immunity 10:281). Illustrative cancer and tumor antigens include, but are not limited to: BRCA1 gene product, BRCA2 gene product, gp100, tyrosinase, GAGE-1/2, BAGE, RAGE, NY-ESO-1, CDK-4, 0-catenin, MUM-1, Caspase-8, KIAA0205, HPVE, SART-1, PRAME, p15, melanoma tumor antigens (Kawakami et al., (1994) Proc. Natl. Acad. Sci. USA 91:3515; Kawakami et al., (1994) J. Exp. Med., 180:347; Kawakami et al., (1994) Cancer Res. 54:3124) including MART-1 (Coulie et al., (1991) J. Exp. Med. 180:35), gp100 (Wick et al., (1988) J. Cutan. Pathol. 4:201) and MAGE antigen (MAGE-1, MAGE-2 and MAGE-3) (Van der Bruggen et al., (1991) Science, 254:1643), CEA, TRβ-1; TRβ-2; β-15 and tyrosinase (Brichard et al., (1993) J. Exp. Med. 178:489); HER-2/neu gene product (U.S. Pat. No. 4,968,603); CA 125; HE4; LK26; FB5 (endosialin); TAG 72; AFP; CA19-9; NSE; DU-PAN-2; CA50; Span-1; CA72-4; HCG; STN (sialyl Tn antigen); c-erbB-2 proteins; PSA; L-CanAg; estrogen receptor; milk fat globulin; p53 tumor suppressor protein (Levine, (1993) Ann. Rev. Biochem. 62:623); mucin antigens (international patent publication WO 90/05142); telomerases; nuclear matrix proteins; prostatic acid phosphatase; papilloma virus antigens; and antigens associated with the following cancers: melanomas, adenocarcinoma, thymoma, sarcoma, lung cancer, liver cancer, colorectal cancer, non-Hodgkin's lymphoma, Hodgkin's lymphoma, leukemias, uterine cancer, breast cancer, prostate cancer, ovarian cancer, cervical cancer, bladder cancer, kidney cancer, pancreatic cancer, brain cancer, kidney cancer, stomach cancer, esophageal cancer, head and neck cancer and others (see, e.g., Rosenberg, (1996) Annu. Rev. Med. 47:481-91).

Alternatively, the heterologous nucleotide sequence may encode any polypeptide that is desirably produced in a cell in vitro, ex vivo, or in vivo. For example, the virus vectors may be introduced into cultured cells and the expressed protein product isolated therefrom.

It will be understood by those skilled in the art that the heterologous nucleic acid(s) of interest may be operably associated with appropriate control sequences. For example, the heterologous nucleic acid may be operably associated with expression control elements, such as transcription/translation control signals, origins of replication, polyadenylation signals, internal ribosome entry sites (IRES), promoters, enhancers, and the like.

Those skilled in the art will further appreciate that a variety of promoter/enhancer elements may be used depending on the level and tissue-specific expression desired. The promoter/enhancer may be constitutive or inducible, depending on the pattern of expression desired. The promoter/enhancer may be native or foreign and can be a natural or a synthetic sequence. By foreign, it is intended that the transcriptional initiation region is not found in the wild-type host into which the transcriptional initiation region is introduced.

Promoter/enhancer elements can be native to the target cell or subject to be treated and/or native to the heterologous nucleic acid sequence. The promoter/enhancer element is generally chosen so that it will function in the target cell(s) of interest. In representative embodiments, the promoter/enhancer element is a mammalian promoter/enhancer element. The promoter/enhance element may be an RNA polymerase II-based promoter or an RNA polymerase III-based promoter. The promoter/enhance element may be constitutive or inducible.

Inducible expression control elements are generally used in those applications in which it is desirable to provide regulation over expression of the heterologous nucleic acid sequence(s). Inducible promoters/enhancer elements for gene delivery can be tissue-specific or tissue-preferred promoter/enhancer elements, and include muscle specific or preferred (including cardiac, skeletal and/or smooth muscle), neural tissue specific or preferred (including brain-specific), eye (including retina-specific and cornea-specific), liver specific or preferred, bone marrow specific or preferred, pancreatic specific or preferred, spleen specific or preferred, and lung specific or preferred promoter/enhancer elements. In one embodiment, a hepatocyte-specific or hepatocyte-preferred promoter is used. Examples of hepatocyte-specific or preferred promoters include, without limitation, apolipoprotein AII, albumin, alpha 1-antitrypsin, thyroxine-binding globulin, cytochrome P450 CYP3A4, or microRNA122 or a synthetic liver-specific regulatory sequence. Use of a hepatocyte-specific or preferred promoter can increase the specificity achieved by the synthetic AAV vector by further limiting expression of the heterologous nucleic acid to the liver. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the heterologous nucleic acid sequence(s) is transcribed and then translated in the target cells, specific initiation signals are generally employed for efficient translation of inserted protein coding sequences. These exogenous translational control sequences, which may include the ATG initiation codon and adjacent sequences, can be of a variety of origins, both natural and synthetic.

The invention also provides synthetic AAV particles comprising an AAV capsid and an AAV genome, wherein the AAV genome “corresponds to” (i.e., encodes) the AAV capsid. Also provided are collections or libraries of such chimeric AAV particles, wherein the collection or library comprises 2 or more, 10 or more, 50 or more, 100 or more, 1000 or more, 104 or more, 105 or more, or 10⁶ or more distinct sequences.

The present invention further encompasses “empty” capsid particles (i.e., in the absence of a vector genome) comprising, consisting of, or consisting essentially of the modified capsid proteins of the invention. The synthetic AAV capsids of the invention can be used as “capsid vehicles,” as has been described in U.S. Pat. No. 5,863,541. Molecules that can be covalently linked, bound to or packaged by the virus capsids and transferred into a cell include DNA, RNA, a lipid, a carbohydrate, a polypeptide, a small organic molecule, or combinations of the same. Further, molecules can be associated with (e.g., “tethered to”) the outside of the virus capsid for transfer of the molecules into host target cells. In one embodiment of the invention the molecule is covalently linked (i.e., conjugated or chemically coupled) to the capsid proteins. Methods of covalently linking molecules are known by those skilled in the art.

The virus capsids of the invention also find use in raising antibodies against the novel capsid structures. As a further alternative, an exogenous amino acid sequence may be inserted into the virus capsid for antigen presentation to a cell, e.g., for administration to a subject to produce an immune response to the exogenous amino acid sequence.

The invention also provides nucleic acids (e.g., isolated nucleic acids) encoding the modified capsid proteins of the invention. Further provided are vectors comprising the nucleic acids, and cells (in vivo or in culture) comprising the nucleic acids and/or vectors of the invention. Such nucleic acids, vectors and cells can be used, for example, as reagents (e.g., helper constructs or packaging cells) for the production of virus vectors as described herein.

In exemplary embodiments, the invention provides nucleic acid sequences encoding the modified capsid of SEQ ID NOS:15-28 or an amino acid sequence at least 90% identical thereto. In some embodiments, the nucleic acid comprises, consists essentially of, or consists of a nucleotide sequence at least 90% identical to the nucleotide sequence of any one of SEQ ID NOS:1-14, e.g., at least 91%, 92%, 93%, 94%, 95%, 96%, 97%, 98%, 99%, 99.5% identical. In some embodiments, the nucleic acid comprises, consists essentially of, or consists of the nucleotide sequence of any one of SEQ ID NOS:1-14.

In some embodiments, the nucleic acid encodes a capsid protein comprising one or more of the following mutations:
a) Q105K;
b) A135G;
c) T179S;
d) I188L;
e) S200P;
f) L201N;
g) D348E;
h) E360Q;
i) D383N;
j) E417K;
k) R459T;
l) H510N;
m) M523I;
n) T526S;
o) P724H;
p) R725H;
q) N735P;
   based on the AAV8 capsid protein sequence or the corresponding residue in another AAV capsid protein.

In some embodiments, the nucleic acid encodes an AAV capsid protein, the nucleic acid comprising an AAV capsid protein coding sequence that encodes a capsid protein comprising one or more mutations in the variable region 1 (VR1) loop and or a chimeric capsid protein in which the VR1 loop has been replaced by a VR1 loop from a capsid protein of a different AAV serotype, wherein an AAV particle comprising the encoded capsid protein has decreased susceptibility to neutralizing antibodies when administered to a subject relative to an AAV particle comprising a wild-type capsid protein, and wherein the VR1 loop corresponds to amino acid residues QISNGTSGGATNDNT (SEQ ID NO:36) in the AAV8 capsid protein and the corresponding amino acids in other serotypes.

In some embodiments, the nucleic acid encodes a capsid protein comprising an E531K mutation.

The invention also provides nucleic acids encoding the capsid protein variants and fusion proteins as described above. In particular embodiments, the nucleic acid hybridizes to the complement of the nucleic acid sequences specifically disclosed herein under standard conditions as known by those skilled in the art and encodes a variant capsid and/or capsid protein. Optionally, the variant capsid or capsid protein substantially retains at least one property of the capsid and/or capsid or capsid protein encoded by the nucleic acid sequence of SEQ ID NOS:1-14. For example, a virus particle comprising the variant capsid or variant capsid protein can substantially retain the Nab evasion and/or the liver tropism profile of a virus particle comprising a capsid or capsid protein encoded by a nucleic acid coding sequence of SEQ ID NO:1-14.

For example, hybridization of such sequences may be carried out under conditions of reduced stringency, medium stringency or even stringent conditions. Exemplary conditions for reduced, medium and stringent hybridization are as follows: (e.g., conditions represented by a wash stringency of 35-40% Formamide with 5×Denhardt's solution, 0.5% SDS and 1×SSPE at 37° C.; conditions represented by a wash stringency of 40-45% Formamide with 5×Denhardt's solution, 0.5% SDS, and 1×SSPE at 42° C.; and conditions represented by a wash stringency of 50% Formamide with 5×Denhardt's solution, 0.5% SDS and 1×SSPE at 42° C., respectively). See, e.g., Sambrook et al., Molecular Cloning, A Laboratory Manual (4th Ed. 2013) (Cold Spring Harbor Laboratory).

In other embodiments, nucleic acid sequences encoding a variant capsid or capsid protein of the invention have at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%, or higher sequence identity with the nucleic acid sequence of SEQ ID NO:1-14 and optionally encode a variant capsid or capsid protein that substantially retains at least one property of the capsid or capsid protein encoded by a nucleic acid of SEQ ID NO:1-14.

As is known in the art, a number of different programs can be used to identify whether a nucleic acid or polypeptide has sequence identity to a known sequence. Percent identity as used herein means that a nucleic acid or fragment thereof shares a specified percent identity to another nucleic acid, when optimally aligned (with appropriate nucleotide insertions or deletions) with the other nucleic acid (or its complementary strand), using BLASTN. To determine percent identity between two different nucleic acids, the percent identity is to be determined using the BLASTN program “BLAST 2 sequences”. This program is available for public use from the National Center for Biotechnology Information (NCBI) over the Internet (Altschul et al., (1997) Nucleic Acids Res. 25(17):3389-3402). The parameters to be used are whatever combination of the following yields the highest calculated percent identity (as calculated below) with the default parameters shown in parentheses: Program—blastn Matrix—0 BLOSUM62 Reward for a match—0 or 1 (1) Penalty for a mismatch—−0, −1, −2 or −3 (−2) Open gap penalty—−0, 1, 2, 3, 4 or 5 (5) Extension gap penalty—−0 or 1 (1) Gap x_dropoff—−0 or 50 (50) Expect—−10.

Percent identity or similarity when referring to polypeptides, indicates that the polypeptide in question exhibits a specified percent identity or similarity when compared with another protein or a portion thereof over the common lengths as determined using BLASTP. This program is also available for public use from the National Center for Biotechnology Information (NCBI) over the Internet (Altschul et al., (1997) Nucleic Acids Res. 25(17):3389-3402). Percent identity or similarity for polypeptides is typically measured using sequence analysis software. See, e.g., the Sequence Analysis Software Package of the Genetics Computer Group, University of Wisconsin Biotechnology Center, 910 University Avenue, Madison, Wis. 53705.

Protein analysis software matches similar sequences using measures of homology assigned to various substitutions, deletions and other modifications. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine.

In particular embodiments, the nucleic acid can comprise, consist essentially of, or consist of a vector including but not limited to a plasmid, phage, viral vector (e.g., AAV vector, an adenovirus vector, a herpesvirus vector, or a baculovirus vector), bacterial artificial chromosome (BAC), or yeast artificial chromosome (YAC). For example, the nucleic acid can comprise, consist of, or consist essentially of an AAV vector comprising a 5′ and/or 3′ terminal repeat (e.g., 5′ and/or 3′ AAV terminal repeat).

In some embodiments, the nucleic acid encoding the synthetic AAV capsid protein further comprises an AAV rep coding sequence. For example, the nucleic acid can be a helper construct for producing viral stocks.

The invention also provides packaging cells stably comprising a nucleic acid of the invention. For example, the nucleic acid can be stably incorporated into the genome of the cell or can be stably maintained in an episomal form (e.g., an “EBV based nuclear episome”).

The nucleic acid can be incorporated into a delivery vector, such as a viral delivery vector. To illustrate, the nucleic acid of the invention can be packaged in an AAV particle, an adenovirus particle, a herpesvirus particle, a baculovirus particle, or any other suitable virus particle.

Moreover, the nucleic acid can be operably associated with a promoter element. Promoter elements are described in more detail herein.

The present invention further provides methods of producing the virus vectors of the invention. In a representative embodiment, the present invention provides a method of producing a recombinant virus vector, the method comprising providing to a cell in vitro, (a) a template comprising (i) a heterologous nucleic acid, and (ii) packaging signal sequences sufficient for the encapsidation of the AAV template into virus particles (e.g., one or more (e.g., two) terminal repeats, such as AAV terminal repeats), and (b) AAV sequences sufficient for replication and encapsidation of the template into viral particles (e.g., the AAV rep and AAV cap sequences encoding an AAV capsid of the invention). The template and AAV replication and capsid sequences are provided under conditions such that recombinant virus particles comprising the template packaged within the capsid are produced in the cell. The method can further comprise the step of collecting the virus particles from the cell. Virus particles may be collected from the medium and/or by lysing the cells.

In one illustrative embodiment, the invention provides a method of producing a rAAV particle comprising an AAV capsid, the method comprising: providing a cell in vitro with a nucleic acid encoding a modified capsid of the invention, an AAV rep coding sequence, an AAV vector genome comprising a heterologous nucleic acid, and helper functions for generating a productive AAV infection; and allowing assembly of the AAV particles comprising the AAV capsid and encapsidating the AAV vector genome.

The cell is typically a cell that is permissive for AAV viral replication. Any suitable cell known in the art may be employed, such as mammalian cells. Also suitable are trans-complementing packaging cell lines that provide functions deleted from a replication-defective helper virus, e.g., 293 cells or other Ela trans-complementing cells.

The AAV replication and capsid sequences may be provided by any method known in the art. Current protocols typically express the AAV rep/cap genes on a single plasmid. The AAV replication and packaging sequences need not be provided together, although it may be convenient to do so. The AAV rep and/or cap sequences may be provided by any viral or non-viral vector. For example, the rep/cap sequences may be provided by a hybrid adenovirus or herpesvirus vector (e.g., inserted into the Ela or E3 regions of a deleted adenovirus vector). EBV vectors may also be employed to express the AAV cap and rep genes. One advantage of this method is that EBV vectors are episomal, yet will maintain a high copy number throughout successive cell divisions (i.e., are stably integrated into the cell as extra-chromosomal elements, designated as an EBV based nuclear episome.

As a further alternative, the rep/cap sequences may be stably carried (episomal or integrated) within a cell.

Typically, the AAV rep/cap sequences will not be flanked by the AAV packaging sequences (e.g., AAV ITRs), to prevent rescue and/or packaging of these sequences.

The template (e.g., an rAAV vector genome) can be provided to the cell using any method known in the art. For example, the template may be supplied by a non-viral (e.g., plasmid) or viral vector. In particular embodiments, the template is supplied by a herpesvirus or adenovirus vector (e.g., inserted into the Ela or E3 regions of a deleted adenovirus). As another illustration, Palombo et al., (1998) J. Virol. 72:5025, describe a baculovirus vector carrying a reporter gene flanked by the AAV ITRs. EBV vectors may also be employed to deliver the template, as described above with respect to the rep/cap genes.

In another representative embodiment, the template is provided by a replicating rAAV virus. In still other embodiments, an AAV provirus is stably integrated into the chromosome of the cell.

To obtain maximal virus titers, helper virus functions (e.g., adenovirus or herpesvirus) essential for a productive AAV infection are generally provided to the cell. Helper virus sequences necessary for AAV replication are known in the art. Typically, these sequences are provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes required for efficient AAV production as described by Ferrari et al., (1997) Nature Med. 3:1295, and U.S. Pat. Nos. 6,040,183 and 6,093,570.

Further, the helper virus functions may be provided by a packaging cell with the helper genes integrated in the chromosome or maintained as a stable extrachromosomal element. In representative embodiments, the helper virus sequences cannot be packaged into AAV virions, e.g., are not flanked by AAV ITRs.

Those skilled in the art will appreciate that it may be advantageous to provide the AAV replication and capsid sequences and the helper virus sequences (e.g., adenovirus sequences) on a single helper construct. This helper construct may be a non-viral or viral construct, but is optionally a hybrid adenovirus or hybrid herpesvirus comprising the AAV rep/cap genes.

In one particular embodiment, the AAV rep/cap sequences and the adenovirus helper sequences are supplied by a single adenovirus helper vector. This vector further contains the rAAV template. The AAV rep/cap sequences and/or the rAAV template may be inserted into a deleted region (e.g., the Ela or E3 regions) of the adenovirus.

In a further embodiment, the AAV rep/cap sequences and the adenovirus helper sequences are supplied by a single adenovirus helper vector. The rAAV template is provided as a plasmid template.

In another illustrative embodiment, the AAV rep/cap sequences and adenovirus helper sequences are provided by a single adenovirus helper vector, and the rAAV template is integrated into the cell as a provirus. Alternatively, the rAAV template is provided by an EBV vector that is maintained within the cell as an extrachromosomal element (e.g., as a “EBV based nuclear episome,” see Margolski, (1992) Curr. Top. Microbiol. Immun. 158:67).

In a further exemplary embodiment, the AAV rep cap sequences and adenovirus helper sequences are provided by a single adenovirus helper. The rAAV template is provided as a separate replicating viral vector. For example, the rAAV template may be provided by a rAAV particle or a second recombinant adenovirus particle.

According to the foregoing methods, the hybrid adenovirus vector typically comprises the adenovirus 5′ and 3′ cis sequences sufficient for adenovirus replication and packaging (i.e., the adenovirus terminal repeats and PAC sequence). The AAV rep cap sequences and, if present, the rAAV template are embedded in the adenovirus backbone and are flanked by the 5′ and 3′ cis sequences, so that these sequences may be packaged into adenovirus capsids. As described above, in representative embodiments, the adenovirus helper sequences and the AAV rep cap sequences are not flanked by the AAV packaging sequences (e.g., the AAV ITRs), so that these sequences are not packaged into the AAV virions.

Herpesvirus may also be used as a helper virus in AAV packaging methods. Hybrid herpesviruses encoding the AAV rep protein(s) may advantageously facilitate for more scalable AAV vector production schemes. A hybrid herpes simplex virus type I (HSV-1) vector expressing the AAV-2 rep and cap genes has been described (Conway et al., (1999) Gene Therapy 6:986 and WO 00/17377, the disclosures of which are incorporated herein in their entireties).

As a further alternative, the virus vectors of the invention can be produced in insect cells using baculovirus vectors to deliver the rep/cap genes and rAAV template as described by Urabe et al., (2002) Human Gene Therapy 13:1935-43.

Other methods of producing AAV use stably transformed packaging cells (see, e.g., U.S. Pat. No. 5,658,785).

AAV vector stocks free of contaminating helper virus may be obtained by any method known in the art. For example, AAV and helper virus may be readily differentiated based on size. AAV may also be separated away from helper virus based on affinity for a heparin substrate (Zolotukhin et al., (1999) Gene Therapy 6:973). In representative embodiments, deleted replication-defective helper viruses are used so that any contaminating helper virus is not replication competent. As a further alternative, an adenovirus helper lacking late gene expression may be employed, as only adenovirus early gene expression is required to mediate packaging of AAV virus. Adenovirus mutants defective for late gene expression are known in the art (e.g., ts100K and ts149 adenovirus mutants).

The inventive packaging methods may be employed to produce high titer stocks of virus particles. In particular embodiments, the virus stock has a titer of at least about 105 transducing units (tu)/ml, at least about 10⁶ tu/ml, at least about 10⁷ tu/ml, at least about 10⁸ tu/ml, at least about 109 tu/ml, or at least about 10¹⁰ tu/ml.

The novel capsid protein and capsid structures find use in raising antibodies, for example, for diagnostic or therapeutic uses or as a research reagent. Thus, the invention also provides antibodies against the novel capsid proteins and capsids of the invention.

The term “antibody” or “antibodies” as used herein refers to all types of immunoglobulins, including IgG, IgM, IgA, IgD, and IgE. The antibody can be monoclonal or polyclonal and can be of any species of origin, including (for example) mouse, rat, rabbit, horse, goat, sheep or human, or can be a chimeric antibody. See, e.g., Walker et al., Mol. Immunol. 26, 403-11 (1989). The antibodies can be recombinant monoclonal antibodies, for example, produced according to the methods disclosed in U.S. Pat. No. 4,474,893 or U.S. Pat. No. 4,816,567. The antibodies can also be chemically constructed, for example, according to the method disclosed in U.S. Pat. No. 4,676,980.

Antibody fragments included within the scope of the present invention include, for example, Fab, F(ab′)2, and Fc fragments, and the corresponding fragments obtained from antibodies other than IgG. Such fragments can be produced by known techniques. For example, F(ab′)2 fragments can be produced by pepsin digestion of the antibody molecule, and Fab fragments can be generated by reducing the disulfide bridges of the F(ab′)2 fragments. Alternatively, Fab expression libraries can be constructed to allow rapid and easy identification of monoclonal Fab fragments with the desired specificity (Huse et al., (1989) Science 254, 1275-1281).

Polyclonal antibodies can be produced by immunizing a suitable animal (e.g., rabbit, goat, etc.) with an antigen to which a monoclonal antibody to the target binds, collecting immune serum from the animal, and separating the polyclonal antibodies from the immune serum, in accordance with known procedures.

Monoclonal antibodies can be produced in a hybridoma cell line according to the technique of Kohler and Milstein, (1975) Nature 265, 495-97. For example, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in E. coli by recombinant techniques known to those skilled in the art. See, e.g., W. Huse, (1989) Science 246, 1275-81.

Antibodies specific to a target polypeptide can also be obtained by phage display techniques known in the art.

Various immunoassays can be used for screening to identify antibodies having the desired specificity. Numerous protocols for competitive binding or immunoradiometric assays using either polyclonal or monoclonal antibodies with established specificity are well known in the art. Such immunoassays typically involve the measurement of complex formation between an antigen and its specific antibody (e.g., antigen/antibody complex formation). A two-site, monoclonal-based immunoassay utilizing monoclonal antibodies reactive to two non-interfering epitopes can be used as well as a competitive binding assay.

Antibodies can be conjugated to a solid support (e.g., beads, plates, slides or wells formed from materials such as latex or polystyrene) in accordance with known techniques. Antibodies can likewise be directly or indirectly conjugated to detectable groups such as radiolabels (e.g., ³⁵S, ¹²⁵I, ¹³¹I), enzyme labels (e.g., horseradish peroxidase, alkaline phosphatase), and fluorescence labels (e.g., fluorescein) in accordance with known techniques. Determination of the formation of an antibody/antigen complex in the methods of this invention can be by detection of, for example, precipitation, agglutination, flocculation, radioactivity, color development or change, fluorescence, luminescence, etc., as is well known in the art.

### Methods of Using Modified Capsids

The present invention also relates to methods for delivering heterologous nucleotide sequences into a subject, e.g., into the liver, while exhibiting low seroreactivity. The present invention may overcome the presence of Nab in many subjects, one of the major hurdles for AAV-based gene therapy.

In one aspect, the invention relates to a method of decreasing the susceptibility of an AAV particle to neutralizing antibodies when administered to a subject, comprising preparing the AAV particle with the modified capsid protein of the invention.

The virus vectors of the invention may be employed to deliver a nucleotide sequence of interest to a hepatocyte or other cell in vitro, e.g., to produce a polypeptide or nucleic acid in vitro or for ex vivo gene therapy. The vectors are additionally useful in a method of delivering a nucleotide sequence to a subject in need thereof, e.g., to express a therapeutic or immunogenic polypeptide or nucleic acid. In this manner, the polypeptide or nucleic acid may thus be produced in vivo in the subject. The subject may be in need of the polypeptide or nucleic acid because the subject has a deficiency of the polypeptide, or because the production of the polypeptide or nucleic acid in the subject may impart some therapeutic effect, as a method of treatment or otherwise, and as explained further below.

In particular embodiments, the vectors are useful to express a polypeptide or nucleic acid that provides a beneficial effect to the subject in general. In other embodiments, the vectors are useful to express a polypeptide or nucleic acid that provides a beneficial effect to cells in the liver (e.g., hepatocytes).

Thus, one aspect of the invention relates to a method of delivering a nucleic acid of interest to a cell, e.g., a hepatocyte, the method comprising contacting the cell, e.g., hepatocyte, with the AAV particle of the invention.

In another aspect, the invention relates to a method of delivering a nucleic acid of interest to a cell, e.g., a hepatocyte, in a mammalian subject, the method comprising administering an effective amount of the AAV particle or pharmaceutical formulation of the invention to a mammalian subject, thereby delivering the nucleic acid of interest to a cell, e.g., hepatocyte, in the mammalian subject.

A further aspect of the invention relates to a method of treating a disorder in a mammalian subject in need thereof, wherein the disorder is treatable by expressing a product in the subject, e.g., in the liver of the subject, the method comprising administering a therapeutically effective amount of the AAV particle of the invention to the subject, wherein the product is expressed, thereby treating the disorder.

In general, the virus vectors of the invention may be employed to deliver any foreign nucleic acid with a biological effect to treat or ameliorate the symptoms associated with any disorder related to gene expression. Further, the invention can be used to treat any disease state for which it is beneficial to deliver a therapeutic polypeptide. Illustrative disease states include, but are not limited to: cystic fibrosis (cystic fibrosis transmembrane regulator protein) and other diseases of the lung, hemophilia A (Factor VIII), hemophilia B (Factor IX), thalassemia (β-globin), anemia (erythropoietin) and other blood disorders, Alzheimer's disease (GDF; neprilysin), multiple sclerosis (β-interferon), Parkinson's disease (glial-cell line derived neurotrophic factor [GDNF]), Huntington's disease (inhibitory RNA including without limitation RNAi such as siRNA or shRNA, antisense RNA or microRNA to remove repeats), amyotrophic lateral sclerosis, epilepsy (galanin, neurotrophic factors), and other neurological disorders, cancer (endostatin, angiostatin, TRAIL, FAS-ligand, cytokines including interferons; inhibitory RNA including without limitation RNAi (such as siRNA or shRNA), antisense RNA and microRNA including inhibitory RNA against VEGF, the multiple drug resistance gene product or a cancer immunogen), diabetes mellitus (insulin, PGC-α1, GLβ-1, myostatin pro-peptide, glucose transporter 4), muscular dystrophies including Duchenne and Becker (e.g., dystrophin, mini-dystrophin, micro-dystrophin, insulin-like growth factor I, a sarcoglycan [e.g., α, β, γ], Inhibitory RNA [e.g., RNAi, antisense RNA or microRNA]against myostatin or myostatin propeptide, laminin-alpha2, Fukutin-related protein, dominant negative myostatin, follistatin, activin type II soluble receptor, anti-inflammatory polypeptides such as the Ikappa B dominant mutant, sarcospan, utrophin, mini-utrophin, inhibitory RNA [e.g., RNAi, antisense RNA or microRNA]against splice junctions in the dystrophin gene to induce exon skipping [see, e.g., WO/2003/095647], inhibitory RNA [e.g., RNAi, antisense RNA or micro RNA]against U7 snRNAs to induce exon skipping [see, e.g., WO/2006/021724], and antibodies or antibody fragments against myostatin or myostatin propeptide), Gaucher disease (glucocerebrosidase), Hurler's disease (α-L-iduronidase), adenosine deaminase deficiency (adenosine deaminase), glycogen storage diseases (e.g., Fabry disease [α-galactosidase] and Pompe disease [lysosomal acid α-glucosidase]) and other metabolic defects including other lysosomal storage disorders and glycogen storage disorders, congenital emphysema (al-antitrypsin), Lesch-Nyhan Syndrome (hypoxanthine guanine phosphoribosyl transferase), Niemann-Pick disease (sphingomyelinase), Maple Syrup Urine Disease (branched-chain keto acid dehydrogenase), retinal degenerative diseases (and other diseases of the eye and retina; e.g., PDGF, endostatin and/or angiostatin for macular degeneration), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver (RNAi such as siRNA or shRNA, microRNA or antisense RNA for hepatitis B and/or hepatitis C genes), kidney, heart including congestive heart failure or peripheral artery disease (PAD) (e.g., by delivering protein phosphatase inhibitor I [I-1], phospholamban, sarcoplasmic endoreticulum Ca-ATPase [serca2a], zinc finger proteins that regulate the phospholamban gene, Pim-1, PGC-1α, SOD-1, SOD-2, ECF-SOD, kallikrein, thymosin-β4, hypoxia-inducible transcription factor [HIF], Parkct, β2-adrenergic receptor, β2-adrenergic receptor kinase [PARK], phosphoinositide-3 kinase [PI3 kinase], calsarcin, an angiogenic factor, S100A1, parvalbumin, adenylyl cyclase type 6, a molecule that effects G-protein coupled receptor kinase type 2 knockdown such as a truncated constitutively active bARKct, an inhibitory RNA [e.g., RNAi, antisense RNA or microRNA] against phospholamban; phospholamban inhibitory or dominant-negative molecules such as phospholamban S16E, etc.), arthritis (insulin-like growth factors), joint disorders (insulin-like growth factors), intimal hyperplasia (e.g., by delivering enos, inos), improve survival of heart transplants (superoxide dismutase), AIDS (soluble CD4), muscle wasting (insulin-like growth factor I, myostatin pro-peptide, an anti-apoptotic factor, follistatin), limb ischemia (VEGF, FGF, PGC-1α, EC-SOD, HIF), kidney deficiency (erythropoietin), anemia (erythropoietin), arthritis (anti-inflammatory factors such as IRAP and TNFα soluble receptor), hepatitis (α-interferon), LDL receptor deficiency (LDL receptor), hyperammonemia (ornithine transcarbamylase), spinal cerebral ataxias including SCA1, SCA2 and SCA3, phenylketonuria (phenylalanine hydroxylase), autoimmune diseases, and the like. The invention can further be used following organ transplantation to increase the success of the transplant and/or to reduce the negative side effects of organ transplantation or adjunct therapies (e.g., by administering immunosuppressant agents or inhibitory nucleic acids to block cytokine production). As another example, bone morphogenic proteins (including RANKL and/or VEGF) can be administered with a bone allograft, for example, following a break or surgical removal in a cancer patient.

Exemplary lysosomal storage diseases that can be treated according to the present invention include without limitation: Hurler's Syndrome (MPS IH), Scheie's Syndrome (MPS IS), and Hurler-Scheie Syndrome (MPS IH/S) (α-L-iduronidase); Hunter's Syndrome (MPS II) (iduronate sulfate sulfatase); Sanfilippo A Syndrome (MPS IIIA) (Heparan-S-sulfate sulfaminidase), Sanfilippo B Syndrome (MPS IIIB) (N-acetyl-D-glucosaminidase), Sanfilippo C Syndrome (MPS IIIC) (Acetyl-CoA-glucosaminide N-acetyltransferase), Sanfilippo D Syndrome (MPS IIID) (N-acetyl-glucosaminine-6-sulfate sulfatase); Morquio A disease (MPS IVA) (Galactosamine-6-sulfate sulfatase), Morquio B disease (MPS IV B) (β-Galactosidase); Maroteaux-1may disease (MPS VI) (arylsulfatase B); Sly Syndrome (MPS VII) (0-glucuronidase); hyaluronidase deficiency (MPS IX) (hyaluronidase); sialidosis (mucolipidosis I), mucolipidosis II (I-Cell disease) (N-actylglucos-aminyl-1-phosphotransferase catalytic subunit), mucolipidosis III (pseudo-Hurler polydystrophy) (N-acetylglucos-aminyl-1-phosphotransferase; type IIIA [catalytic subunit] and type IIIC [substrate recognition subunit]); GM1 gangliosidosis (ganglioside β-galactosidase), GM2 gangliosidosis Type I (Tay-Sachs disease) (p-hexaminidase A), GM2 gangliosidosis type II (Sandhoff's disease) (p-hexosaminidase B); Niemann-Pick disease (Types A and B) (sphingomyelinase); Gaucher's disease (glucocerebrosidase); Farber's disease (ceraminidase); Fabry's disease (α-galactosidase A); Krabbe's disease (galactosylceramide β-galactosidase); metachromatic leukodystrophy (arylsulfatase A); lysosomal acid lipase deficiency including Wolman's disease (lysosomal acid lipase); Batten disease (juvenile neuronal ceroid lipofuscinosis) (lysosomal trans-membrane CLN3 protein) sialidosis (neuraminidase 1); galactosialidosis (Goldberg's syndrome) (protective protein/cathepsin A); α-mannosidosis (α-D-mannosidase); 0-mannosidosis (β-D-mannosidosis); fucosidosis (α-D-fucosidase); aspartylglucosaminuria (N-Aspartylglucosaminidase); and sialuria (Na phosphate cotransporter).

Exemplary glycogen storage diseases that can be treated according to the present invention include, but are not limited to, Type Ia GSD (von Gierke disease) (glucose-6-phosphatase), Type Ib GSD (glucose-6-phosphate translocase), Type Ic GSD (microsomal phosphate or pyrophosphate transporter), Type Id GSD (microsomal glucose transporter), Type II GSD including Pompe disease or infantile Type IIa GSD (lysosomal acid α-glucosidase) and Type IIb (Danon) (lysosomal membrane protein-2), Type IIIa and IIIb GSD (Debrancher enzyme; amyloglucosidase and oligoglucanotransferase), Type IV GSD (Andersen's disease) (branching enzyme), Type V GSD (McArdle disease) (muscle phosphorylase), Type VI GSD (Hers' disease) (liver phosphorylase), Type VII GSD (Tarui's disease) (phosphofructokinase), GSD Type VIII/IXa (X-linked phosphorylase kinase), GSD Type IXb (Liver and muscle phosphorylase kinase), GSD Type IXc (liver phosphorylase kinase), GSD Type IXd (muscle phosphorylase kinase), GSD O (glycogen synthase), Fanconi-Bickel syndrome (glucose transporter-2), phosphoglucoisomerase deficiency, muscle phosphoglycerate kinase deficiency, phosphoglycerate mutase deficiency, fructose 1,6-diphosphatase deficiency, phosphoenolpyruvate carboxykinase deficiency, and lactate dehydrogenase deficiency.

Gene transfer has substantial potential use in understanding and providing therapy for disease states. There are a number of inherited diseases in which defective genes are known and have been cloned. In general, the above disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using inhibitory RNA such as RNAi (e.g., siRNA or shRNA), microRNA or antisense RNA. For unbalanced disease states, gene transfer can be used to create a disease state in a model system, which can then be used in efforts to counteract the disease state. Thus, the virus vectors according to the present invention permit the treatment of genetic diseases. As used herein, a disease state is treated by partially or wholly remedying the deficiency or imbalance that causes the disease or makes it more severe. The use of site-specific recombination of nucleic sequences to cause mutations or to correct defects is also possible.

The virus vectors according to the present invention may also be employed to provide an antisense nucleic acid or inhibitory RNA (e.g., microRNA or RNAi such as a siRNA or shRNA) to a cell in vitro or in vivo. Expression of the inhibitory RNA in the target cell diminishes expression of a particular protein(s) by the cell. Accordingly, inhibitory RNA may be administered to decrease expression of a particular protein in a subject in need thereof. Inhibitory RNA may also be administered to cells in vitro to regulate cell physiology, e.g., to optimize cell or tissue culture systems.

As a further aspect, the virus vectors of the present invention may be used to produce an immune response in a subject. According to this embodiment, a virus vector comprising a nucleic acid encoding an immunogen may be administered to a subject, and an active immune response (optionally, a protective immune response) is mounted by the subject against the immunogen. Immunogens are as described hereinabove.

Alternatively, the virus vector may be administered to a cell ex vivo and the altered cell is administered to the subject. The heterologous nucleic acid is introduced into the cell, and the cell is administered to the subject, where the heterologous nucleic acid encoding the immunogen is optionally expressed and induces an immune response in the subject against the immunogen. In particular embodiments, the cell is an antigen-presenting cell (e.g., a dendritic cell).

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to immunogens by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “protective” immune response or “protective” immunity as used herein indicates that the immune response confers some benefit to the subject in that it prevents or reduces the incidence of disease. Alternatively, a protective immune response or protective immunity may be useful in the treatment of disease, in particular cancer or tumors (e.g., by causing regression of a cancer or tumor and/or by preventing metastasis and/or by preventing growth of metastatic nodules). The protective effects may be complete or partial, as long as the benefits of the treatment outweigh any disadvantages thereof.

The virus vectors of the present invention may also be administered for cancer immunotherapy by administration of a viral vector expressing a cancer cell antigen (or an immunologically similar molecule) or any other immunogen that produces an immune response against a cancer cell. To illustrate, an immune response may be produced against a cancer cell antigen in a subject by administering a viral vector comprising a heterologous nucleotide sequence encoding the cancer cell antigen, for example to treat a patient with cancer. The virus vector may be administered to a subject in vivo or by using ex vivo methods, as described herein.

As used herein, the term “cancer” encompasses tumor-forming cancers. Likewise, the term “cancerous tissue” encompasses tumors. A “cancer cell antigen” encompasses tumor antigens.

The term “cancer” has its understood meaning in the art, for example, an uncontrolled growth of tissue that has the potential to spread to distant sites of the body (i.e., metastasize). Exemplary cancers include, but are not limited to, leukemia, lymphoma (e.g., Hodgkin and non-Hodgkin lymphomas), colorectal cancer, renal cancer, liver cancer, breast cancer, lung cancer, prostate cancer, testicular cancer, ovarian cancer, uterine cancer, cervical cancer, brain cancer (e.g., gliomas and glioblastoma), bone cancer, sarcoma, melanoma, head and neck cancer, esophageal cancer, thyroid cancer, and the like. In embodiments of the invention, the invention is practiced to treat and/or prevent tumor-forming cancers.

The term “tumor” is also understood in the art, for example, as an abnormal mass of undifferentiated cells within a multicellular organism. Tumors can be malignant or benign. In representative embodiments, the methods disclosed herein are used to prevent and treat malignant tumors.

Cancer cell antigens have been described hereinabove. By the terms “treating cancer” or “treatment of cancer,” it is intended that the severity of the cancer is reduced or the cancer is prevented or at least partially eliminated. For example, in particular contexts, these terms indicate that metastasis of the cancer is prevented or reduced or at least partially eliminated. In further representative embodiments these terms indicate that growth of metastatic nodules (e.g., after surgical removal of a primary tumor) is prevented or reduced or at least partially eliminated. By the terms “prevention of cancer” or “preventing cancer” it is intended that the methods at least partially eliminate or reduce the incidence or onset of cancer. Alternatively stated, the onset or progression of cancer in the subject may be slowed, controlled, decreased in likelihood or probability, or delayed.

In particular embodiments, cells may be removed from a subject with cancer and contacted with a virus vector according to the present invention. The modified cell is then administered to the subject, whereby an immune response against the cancer cell antigen is elicited. This method is particularly advantageously employed with immunocompromised subjects that cannot mount a sufficient immune response in vivo (i.e., cannot produce enhancing antibodies in sufficient quantities).

It is known in the art that immune responses may be enhanced by immunomodulatory cytokines (e.g., α-interferon, 0-interferon, y-interferon, co-interferon, τ-interferon, interleukin-1α, interleukin-1β, interleukin-2, interleukin-3, interleukin-4, interleukin 5, interleukin-6, interleukin-7, interleukin-8, interleukin-9, interleukin-10, interleukin-11, interleukin 12, interleukin-13, interleukin-14, interleukin-18, B cell Growth factor, CD40 Ligand, tumor necrosis factor-α, tumor necrosis factor-β, monocyte chemoattractant protein-1, granulocyte-macrophage colony stimulating factor, and lymphotoxin). Accordingly, immunomodulatory cytokines (e.g., CTL inductive cytokines) may be administered to a subject in conjunction with the virus vectors.

Cytokines may be administered by any method known in the art. Exogenous cytokines may be administered to the subject, or alternatively, a nucleotide sequence encoding a cytokine may be delivered to the subject using a suitable vector, and the cytokine produced in vivo.

The viral vectors are further useful for targeting liver cells for research purposes, e.g., for study of liver function in vitro or in animals or for use in creating and/or studying animal models of disease. For example, the vectors can be used to deliver heterologous nucleic acids to hepatocytes in animal models of liver injury, e.g., fibrosis or cirrhosis or animal models of liver diseases such as viral infections (e.g., hepatitis viruses).

Further, the virus vectors according to the present invention find further use in diagnostic and screening methods, whereby a gene of interest is transiently or stably expressed in a cell culture system, or alternatively, a transgenic animal model. The invention can also be practiced to deliver a nucleic acid for the purposes of protein production, e.g., for laboratory, industrial or commercial purposes.

Recombinant virus vectors according to the present invention find use in both veterinary and medical applications. Suitable subjects include both avians and mammals. The term “avian” as used herein includes, but is not limited to, chickens, ducks, geese, quail, turkeys, pheasant, parrots, parakeets. The term “mammal” as used herein includes, but is not limited to, humans, primates non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. Optionally, the subject is “in need of” the methods of the present invention, e.g., because the subject has or is believed at risk for a disorder including those described herein or that would benefit from the delivery of a nucleic acid including those described herein. As a further option, the subject can be a laboratory animal and/or an animal model of disease.

In particular embodiments, the present invention provides a pharmaceutical composition comprising a virus vector of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and will preferably be in solid or liquid particulate form.

By “pharmaceutically acceptable” it is meant a material that is not toxic or otherwise undesirable, i.e., the material may be administered to a subject without causing any undesirable biological effects.

One aspect of the present invention is a method of transferring a nucleic acid to a cell in vitro. The virus vector may be introduced to the cells at the appropriate multiplicity of infection according to standard transduction methods appropriate for the particular target cells. Titers of the virus vector or capsid to administer can vary, depending upon the target cell type and number, and the particular virus vector or capsid, and can be determined by those of skill in the art without undue experimentation. In particular embodiments, at least about 10³ infectious units, more preferably at least about 105 infectious units are introduced to the cell.

The cell(s) into which the virus vector can be introduced may be of any type, including but not limited to neural cells (including cells of the peripheral and central nervous systems, in particular, brain cells such as neurons, oligodendrocytes, glial cells, astrocytes), lung cells, cells of the eye (including retinal cells, retinal pigment epithelium, and corneal cells), epithelial cells (e.g., gut and respiratory epithelial cells), skeletal muscle cells (including myoblasts, myotubes and myofibers), diaphragm muscle cells, dendritic cells, pancreatic cells (including islet cells), hepatic cells, a cell of the gastrointestinal tract (including smooth muscle cells, epithelial cells), heart cells (including cardiomyocytes), bone cells (e.g., bone marrow stem cells), hematopoietic stem cells, spleen cells, keratinocytes, fibroblasts, endothelial cells, prostate cells, joint cells (including, e.g., cartilage, meniscus, synovium and bone marrow), germ cells, and the like. Alternatively, the cell may be any progenitor cell. As a further alternative, the cell can be a stem cell (e.g., neural stem cell, liver stem cell). As still a further alternative, the cell may be a cancer or tumor cell (cancers and tumors are described above). Moreover, the cells can be from any species of origin, as indicated above.

The virus vectors may be introduced to cells in vitro for the purpose of administering the modified cell to a subject. In particular embodiments, the cells have been removed from a subject, the virus vector is introduced therein, and the cells are then replaced back into the subject. Methods of removing cells from subject for treatment ex vivo, followed by introduction back into the subject are known in the art (see, e.g., U.S. Pat. No. 5,399,346). Alternatively, the recombinant virus vector is introduced into cells from another subject, into cultured cells, or into cells from any other suitable source, and the cells are administered to a subject in need thereof.

Suitable cells for ex vivo gene therapy are as described above. Dosages of the cells to administer to a subject will vary upon the age, condition and species of the subject, the type of cell, the nucleic acid being expressed by the cell, the mode of administration, and the like. Typically, at least about 10² to about 10⁸ or about 10³ to about 10⁶ cells will be administered per dose in a pharmaceutically acceptable carrier. In particular embodiments, the cells transduced with the virus vector are administered to the subject in an effective amount in combination with a pharmaceutical carrier.

In some embodiments, cells that have been transduced with the virus vector may be administered to elicit an immunogenic response against the delivered polypeptide (e.g., expressed as a transgene or in the capsid). Typically, a quantity of cells expressing an effective amount of the polypeptide in combination with a pharmaceutically acceptable carrier is administered. Optionally, the dosage is sufficient to produce a protective immune response (as defined above). The degree of protection conferred need not be complete or permanent, as long as the benefits of administering the immunogenic polypeptide outweigh any disadvantages thereof.

A further aspect of the invention is a method of administering the virus vectors or capsids of the invention to subjects. In particular embodiments, the method comprises a method of delivering a nucleic acid of interest to an animal subject, the method comprising: administering an effective amount of a virus vector according to the invention to an animal subject. Administration of the virus vectors of the present invention to a human subject or an animal in need thereof can be by any means known in the art. Optionally, the virus vector is delivered in an effective dose in a pharmaceutically acceptable carrier.

The virus vectors of the invention can further be administered to a subject to elicit an immunogenic response (e.g., as a vaccine). Typically, vaccines of the present invention comprise an effective amount of virus in combination with a pharmaceutically acceptable carrier. Optionally, the dosage is sufficient to produce a protective immune response (as defined above). The degree of protection conferred need not be complete or permanent, as long as the benefits of administering the immunogenic polypeptide outweigh any disadvantages thereof. Subjects and immunogens are as described above.

Dosages of the virus vectors to be administered to a subject will depend upon the mode of administration, the disease or condition to be treated, the individual subject's condition, the particular virus vector, and the nucleic acid to be delivered, and can be determined in a routine manner. Exemplary doses for achieving therapeutic effects are virus titers of at least about 10⁵, 10⁶, 107, 10⁸, 10⁹, 10¹⁰, 10¹¹, 10¹², 10¹³, 10¹⁴, 10¹⁵, 10¹⁶, 10¹⁷, 10¹⁸ transducing units or more, preferably about 10⁷ or 10⁸, 10⁹, 10¹⁰, 10¹¹, 10¹², 10¹³, 10¹⁴ or 10¹⁵ transducing units, yet more preferably about 10¹² to 10¹⁴ transducing units.

In particular embodiments, more than one administration (e.g., two, three, four or more administrations) may be employed to achieve the desired level of gene expression over a period of various intervals, e.g., daily, weekly, monthly, yearly, etc.

Exemplary modes of administration include oral, rectal, transmucosal, topical, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intro-lymphatic, and the like, as well as direct tissue or organ injection (e.g., to liver, skeletal muscle, cardiac muscle, diaphragm muscle or brain). Administration can also be to a tumor (e.g., in or a near a tumor or a lymph node). The most suitable route in any given case will depend on the nature and severity of the condition being treated and on the nature of the particular vector that is being used.

In some embodiments, the viral vector is administered directly to the liver. Direct administration can result in high specificity of transduction of hepatocytes, e.g., wherein at least 80%, 85%, 90%, 95% or more of the transduced cells are hepatocytes. Any method known in the art to administer vectors directly to the liver can be used. The vector may be introduced by direct injection into the liver or injection into an artery or vein feeding the liver, e.g., intraportal delivery.

Typically, the viral vector will be administered in a liquid formulation by systemic delivery or direct injection to the desired region or compartment in the liver. In some embodiments, the vector can be delivered via a reservoir and/or pump. In other embodiments, the vector may be provided by topical application to the desired region or by intra-nasal administration of an aerosol formulation. Administration to the eye or into the ear, may be by topical application of liquid droplets. As a further alternative, the vector may be administered as a solid, slow-release formulation. Controlled release of parvovirus and AAV vectors is described by international patent publication WO 01/91803.

Delivery to any of these tissues can also be achieved by delivering a depot comprising the virus vector, which can be implanted into the tissue or the tissue can be contacted with a film or other matrix comprising the virus vector. Examples of such implantable matrices or substrates are described in U.S. Pat. No. 7,201,898).

Injectables can be prepared in conventional forms, either as liquid solutions or suspensions, solid forms suitable for solution or suspension in liquid prior to injection, or as emulsions. Alternatively, one may administer the virus vector in a local rather than systemic manner, for example, in a depot or sustained-release formulation. Further, the virus vector can be delivered dried to a surgically implantable matrix such as a bone graft substitute, a suture, a stent, and the like (e.g., as described in U.S. Pat. No. 7,201,898).

Pharmaceutical compositions suitable for oral administration can be presented in discrete units, such as capsules, cachets, lozenges, or tablets, each containing a predetermined amount of the composition of this invention; as a powder or granules; as a solution or a suspension in an aqueous or non-aqueous liquid; or as an oil-in-water or water-in-oil emulsion. Oral delivery can be performed by complexing a virus vector of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the composition and a suitable carrier (which may contain one or more accessory ingredients as noted above). In general, the pharmaceutical composition according to embodiments of the present invention are prepared by uniformly and intimately admixing the composition with a liquid or finely divided solid carrier, or both, and then, if necessary, shaping the resulting mixture. For example, a tablet can be prepared by compressing or molding a powder or granules containing the composition, optionally with one or more accessory ingredients. Compressed tablets are prepared by compressing, in a suitable machine, the composition in a free-flowing form, such as a powder or granules optionally mixed with a binder, lubricant, inert diluent, and/or surface active/dispersing agent(s). Molded tablets are made by molding, in a suitable machine, the powdered compound moistened with an inert liquid binder.

Pharmaceutical compositions suitable for buccal (sub-lingual) administration include lozenges comprising the composition of this invention in a flavored base, usually sucrose and acacia or tragacanth; and pastilles comprising the composition in an inert base such as gelatin and glycerin or sucrose and acacia.

Pharmaceutical compositions suitable for parenteral administration can comprise sterile aqueous and non-aqueous injection solutions of the composition of this invention, which preparations are optionally isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The compositions can be presented in unit/dose or multi-dose containers, for example, in sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, an injectable, stable, sterile composition of this invention in a unit dosage form in a sealed container can be provided. The composition can be provided in the form of a lyophilizate, which can be reconstituted with a suitable pharmaceutically acceptable carrier to form a liquid composition suitable for injection into a subject. The unit dosage form can be from about 1 μg to about 10 grams of the composition of this invention. When the composition is substantially water-insoluble, a sufficient amount of emulsifying agent, which is physiologically acceptable, can be included in sufficient quantity to emulsify the composition in an aqueous carrier. One such useful emulsifying agent is phosphatidyl choline.

Pharmaceutical compositions suitable for rectal administration can be presented as unit dose suppositories. These can be prepared by admixing the composition with one or more conventional solid carriers, such as for example, cocoa butter and then shaping the resulting mixture.

Pharmaceutical compositions of this invention suitable for topical application to the skin can take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical composition of the present invention with a lipophilic reagent (e.g., DMSO) that is capable of passing into the skin.

Pharmaceutical compositions suitable for transdermal administration can be in the form of discrete patches adapted to remain in intimate contact with the epidermis of the subject for a prolonged period of time. Compositions suitable for transdermal administration can also be delivered by iontophoresis (see, for example, Pharm. Res. 3:318 (1986)) and typically take the form of an optionally buffered aqueous solution of the composition of this invention. Suitable formulations can comprise citrate or bis\tris buffer (pH 6) or ethanol/water and can contain from 0.1 to 0.2M active ingredient.

The virus vectors disclosed herein may be administered to the lungs of a subject by any suitable means, for example, by administering an aerosol suspension of respirable particles comprised of the virus vectors, which the subject inhales. The respirable particles may be liquid or solid. Aerosols of liquid particles comprising the virus vectors may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the virus vectors may likewise be produced with any solid particulate medicament aerosol generator, by techniques known in the pharmaceutical art.

Having described the present invention, the same will be explained in greater detail in the following examples, which are included herein for illustration purposes only, and which are not intended to be limiting to the invention.

### Example 1

### Methods and Materials

Human sera: Normal human serum samples were purchased from Valley Biomedical (Winchester, VA). The sera from hemophilia patients were collected in The Hemophilia Treatment Center of School of Medicine at UNC-Chapel Hill (UNC IRB #15-2126). Human IVIG was purchased from Grifols Therapeutics Inc. (Research Triangle Park, NC). All the sera and IVIG solution were aliquoted and stored in −80° C.

Cells and mice: HEK293 and Huh7 cell lines were incubated at 37° C. in 5% CO2 in Dulbecco's modified Eagle's medium (DMEM) supplemented with 10% fetal bovine serum (FBS) and penicillin-streptomycin.

Chimeric mice xenografted with human hepatocytes with 70% repopulation were purchased from Yecuris (Tualatin, OR). Mice were maintained in a specific pathogen-free facility at the University of North Carolina at Chapel Hill. All procedures were approved by the University of North Carolina Institutional Animal Care and Use Committee. 4-6-month-old female mice were used.

AAV vector production: AAV vector was produced using triple plasmid transfection. Briefly, HEK-293 cells were transfected with an AAV transgene plasmid (single-stranded (ss) pTR-CBA-Luciferase, or self-complementary (sc) pTR-CBh-GFP), an AAV helper plasmid (the rep and cap genes), and the adenovirus helper plasmid pXX6-80. Forty-eight hours after transfection, HEK-293 cells were harvested, lysed, and AAV vector purification was performed by cesium chloride (CsCl) gradient density centrifugation. The virus titer was determined by Q-PCR.

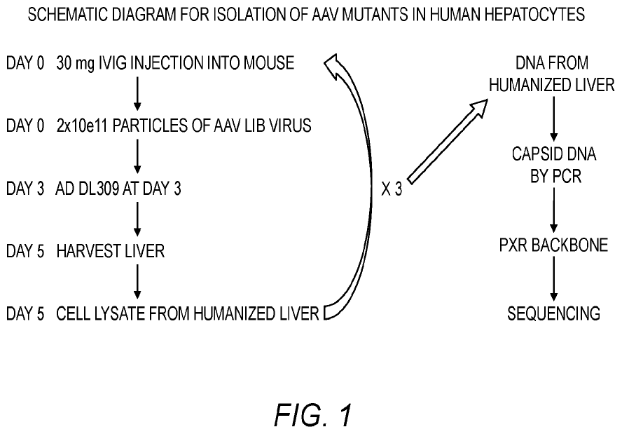

Isolation of AAV mutants in human hepatocytes: The AAV library which consisted of shuffled capsids from serotypes 1-6, 8, 9, and an AAV8 with E531K mutation was produced as described previously (Gray et al., Mol. Ther. 18:570 (2010); Bartel et al., Gene Ther. 19:694 (2012); Chai et al., Bio. Protoc. 8 (2018)). The humanized mice were administered 30 mg human IVIG via retro-orbital injection. Four hours later, 2×10¹¹ particles of AAV shuffled library were intravenously injected. At 3 days after AAV injection, Ad virus d1309 was intravenously injected at 2×10⁹ MOI. Two days later, mice were euthanized and the livers were harvested and lysed. The cell lysate was generated by three freeze-thaw cycles, heated to 56° C. for 30 min to inactivate adenovirus, and then clarified by centrifugation to remove cellular debris. The pooled supernatant of cell lysate was used to infect human hepatocytes in other humanized mice treated with 30 mg human IVIG, then Ad d1309 viruses were supplied for further amplification of AAV capsids. This process was repeated for two more cycles (FIG. 1). Following the final cycle, capsid DNA sequences from isolated liver DNA were amplified through PCR and cloned into the pXR shuttle vector. Individual clones, each potentially representing a chimeric capsid DNA sequence, were sequenced and recombinant vectors packaged with GFP and luciferase were generated for in vitro and in vivo characterization.

Neutralizing antibody assay: Huh7 cells were plated with 1×10⁵ cells per well in a 48 well plate. 1×10⁹ particles of AAV/LUC vectors were incubated with IVIG or sera from healthy subjects or hemophilia patients at different dilutions for 1 hr at 4° C., then added into Huh7 cell culture medium. After culture for 48 hr, luciferase activity in cell lysate was measured with a Wallac-1420 Victor 2 automated plate reader.

Human hepatocyte transduction with AAV vectors in chimeric mice: All mice received retro-orbital injection of 1×10¹¹ particles of self-complementary AAV/GFP vectors. Three weeks later, mice were euthanized and livers were harvested. Part of liver tissues was fixed in 10% formalin for immunohistochemistry staining. Another part of liver tissues was used to prepare single hepatocytes for flow cytometry analysis.

Immunohistochemistry: As described before (Shao et al., Gene Ther. 26:504 (2019)), after fixation, liver tissues were embedded in paraffin and sectioned. The liver sections were rehydrated with serial concentrations of ethanol, and then antigen was retrieved. After blocking, liver tissues were incubated with a primary antibody goat anti-human albumin and then the Alexa Flour 488-conjugated secondary antibody. After washing, liver tissues were stained with chicken anti-GFP antibody followed by the Alexa Flour 594-conjugated secondary antibody. Finally, the tissues were stained with DAPI.

Flow cytometry analysis: Single-cell suspensions from liver tissues were fixed and permeabilized with Fixation/Permeabilization solution (BD), and then incubated with goat anti human albumin antibody (Bethyl) for 30 min. After washing with cold PBS, cells were stained with APC conjugated donkey anti goat IgG antibody (R&D systerm). After washing with PBS, cells were analyzed by flow cytometry.

Molecular modeling: Wild type AAV cryo-structures were obtained from PDB (Protein Data Bank), and PDBs for AAV2, 6, 8 and 9 were 6IHB, 3SHM, 3RA2 and 6NXE, respectively. After aligning with the wild type capsids, the capsid amino acid sequence of AAV LP2-10 which was composed of fragments from AAV2, 6, 8 and 9 was re-assembled according to the DNA sequencing result by Chimera (UCSF).

Phylogenetic analysis: ClustalW was used to align the entire capsid aa sequences of all AAV mutants and wild serotypes (Thompson et al., Nucleic Acids Res. 22:4673 (1994)), and the MEGAv5.05 was applied to generate phylogenetic trees (Tamura et al., Mol. Biol. Evolution 28:2731 (2011)).

Statistics: The Student's t-test (paired) was used to perform statistical analysis. P<0.05 was considered a statistically significant difference. * indicated p<0.05, ** indicated p<0.01; *** indicated p<0.005.

### Example 2

### Characterization of Isolated AAV Mutants from Human Hepatocytes

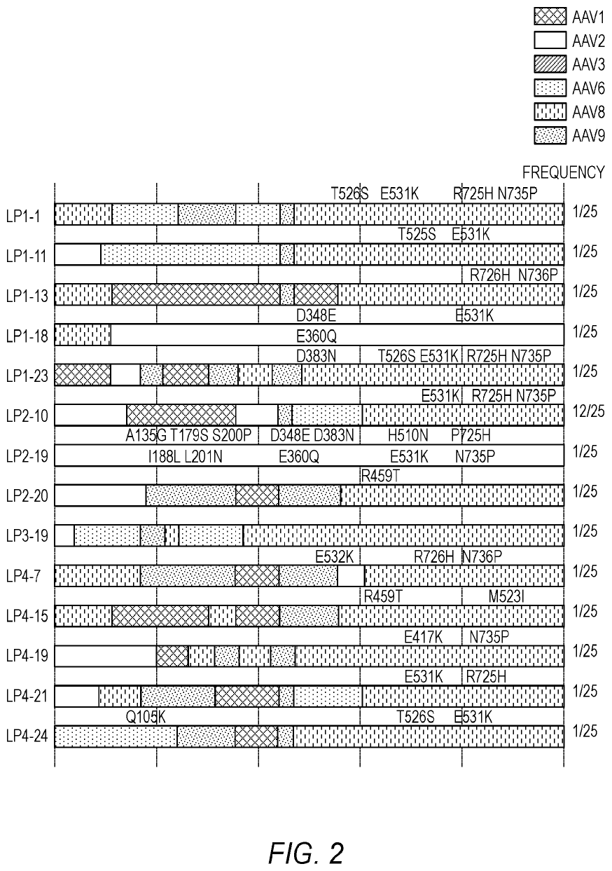

To isolate AAV variants with the ability to escape Nabs and remain human hepatocyte tropism, an in vivo selection was performed in the presence of Nabs in chimeric humanized mice xenografted with human hepatocytes (FIG. 1). After adoptively transferring 30 mg of human IVIG in mice to generate Nabs in circulation, mice were administered with 2×10¹¹ particles of AAV shuffled library, then adenovirus d1309 was applied to increase AAV genome replication and virion assembly in human hepatocytes. Three days later, mouse livers were collected for generation of cell lysate. The cell lysate was used to infect mice with pre-IVIG treatment for second cycle of AAV amplification in human hepatocytes as described above. Such amplification was repeated two more cycles, and then livers were harvested for DNA extraction. PCR for AAV capsid DNA was performed and its products were cloned into pXR plasmids. The capsid DNA in different clones was sequenced. A total of 25 validated clones were available; one capsid AAV LP2-10 was found in 12 clones, the other 13 capsids were only detected in single clones. Except for two capsids (AAV LP2-20 and AAV LP4-15) with the c-terminus from AAV2, all other capsid mutants had C-terminus derived from AAV8 with different point mutations (FIG. 2). Seven mutants had the AAV2 N-terminus, 5 mutants had AAV8 N-terminal sequences, and the N-terminus of the other 2 mutants were derived from AAV1 and AAV3. Specific for mutant AAV LP2-10, the capsid was composed of domains from AAV2, AAV6, AAV8 and AAV9. Its VP3 was from AAV8 with swap of AAV6 from residue 261 to residue 272 (FIG. 2). The mutation E531K on AAV8 capsid was reserved in most clones.

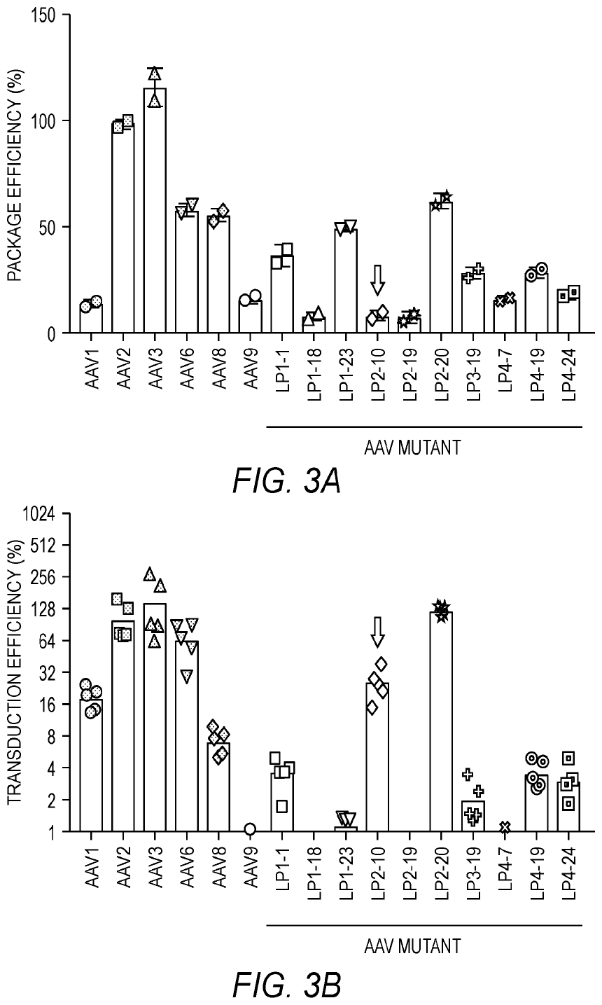

Next, the virus production efficiency from these mutants was studied. As shown in FIGS. 3A-3B, four mutants (AAV LP1-11, LP1-13, LP4-15 and LP4-21) rarely made AAV vectors. When compared to AAV2 virus yield, the virus titer was a little lower from some mutants including AAV LP2-10, but similar to AAV1 and AAV9 (FIG. 3A). Next, these viruses were used to transduce Huh 7 cells. Generally, most viruses had lower transduction than AAV2, 3 and 6, but similar to or higher than AAV9. Except for 3 mutants (AAV LP1-18, LP2-19 and LP4-7), other selected capsids induced a similar transduction to AAV8 (FIG. 3B).

### Example 3

### Nab Evasion Capacity of AAV Mutants for Human IVIG

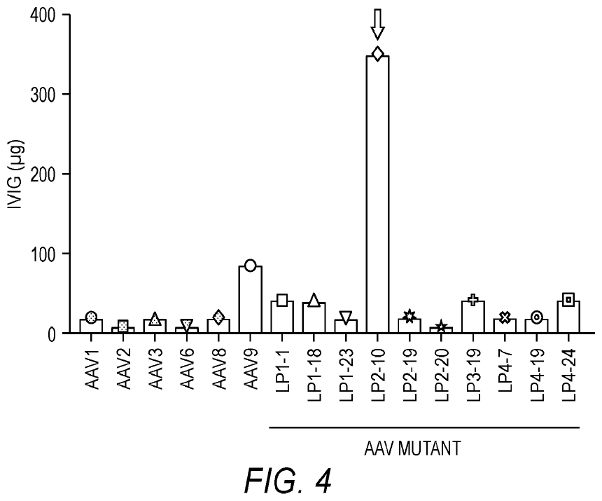

Since these mutants were isolated from the liver of humanized mice in the presence of human IVIG, we then studied the pattern of neutralizing activity of IVIG against these mutant capsids. Not surprisingly, a very low concentration of IVIG was able to inhibit transduction from AAV2 and AAV6 followed by AAV1, AAV3 and AAV8, more IVIG was needed to block AAV9 transduction (FIG. 4). Most of the mutants had a similar Nab profile to AAV8. Mutant AAV LP2-20 had the lowest capacity to evade IVIG Nab activity similar to AAV2. Strikingly, mutant AAV LP2-10 showed the highest ability to escape IVIG neutralizing activity, needing much more IVIG to block its transduction than AAV9 (FIG. 4). This result indicates that AAV mutants can be isolated from humanized mouse livers in the presence of Nabs and are able to escape the cognate Nab activity.

### Example 4

### The Profile of Nabs from Normal Human Serum Against AAV Mutants Isolated from Chimeric Mouse Livers

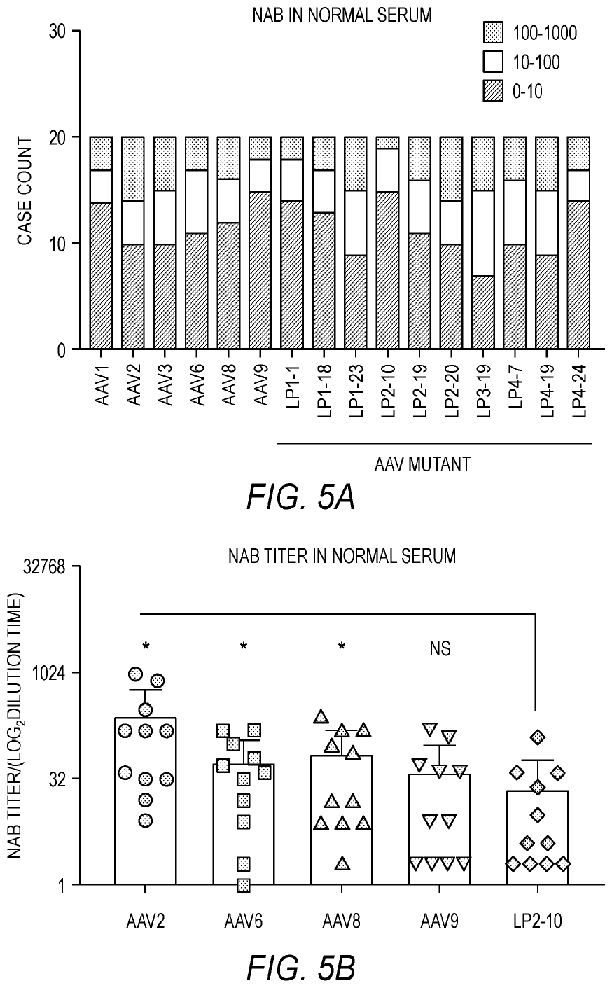

The results described above demonstrated that mutant AAV LP2-10 had a significant capability to resist the neutralizing antibody activity of human IVIG. Next, it was investigated whether these mutants isolated from the liver of humanized mice had different Nab profiles for individual healthy subjects. Twenty serum samples from the healthy population were tested. Generally, the prevalence of Nabs to AAV2 and AAV3 was high (FIG. 5A and Table 3). The titer of Nabs to AAV9 was the lowest among the tested AAV serotypes. The pattern of Nabs for some AAV mutants were similar to AAV9. The Nab titer for AAV LP2-10 was lower than 1:20 dilution in the majority of subjects. A Nab titer over 1:100 was only observed in one subject (FIG. 5A). Since mutant AAV LP2-10 had the capsids derived from AAV2, 6, 8, and 9, the prevalence of Nabs in healthy human subjects was compared for mutant AAV LP2-10 and these four parental AAV serotypes. As shown in FIG. 5B, when compared to AAV2, 6, and 8, mutant AAV LP2-10 has significantly lower Nab titer in healthy individuals (p<0.05). However, mutant AAV LP2-10 had similar low Nab titer to AAV9 (FIG. 5B).

### Example 5

### The Profile of Nabs from Hemophilia Patients' Serum Against AAV Mutants Isolated from Chimeric Mouse Livers

AAV vector mediated gene delivery has been successfully applied in patients with hemophilia via liver targeting. To explore the profile of Nabs against AAV mutants isolated from human hepatocytes in the presence of human IVIG, the Nab analysis was performed in 26 patients with hemophilia (FIG. 5C and Table 4). Similar to healthy subjects, Nabs with high titers (over 1:20) were observed in AAV2 and AAV3 followed by AAV6 and AAV8. For some AAV mutants (AAV LP1-1, LP1-18, LP2-10, LP4-7 and LP4-24), no Nabs with titer over 1:100 were found. Specific for AAV LP2-10, only 8 patients had Nabs (>1:2) to LP2-10. The case number with Nabs was increased to 8, 19, 24 and 14 for AAV1, AAV2, AAV8 and AAV9, respectively. Similar to the result in healthy subjects, generally, the significant low titer of Nabs for mutant AAV LP2-10 was detected when compared to AAV serotypes except for AAV9 (FIG. 5D). These studies from Nab analysis for IVIG or sera of humans indicate that AAV mutants with the capacity to evade Nabs can be isolated from human hepatocytes of chimeric mice.

### Example 6

### Human Hepatocyte Transduction with Mutant AAV LP2-10 in Chimeric Mice

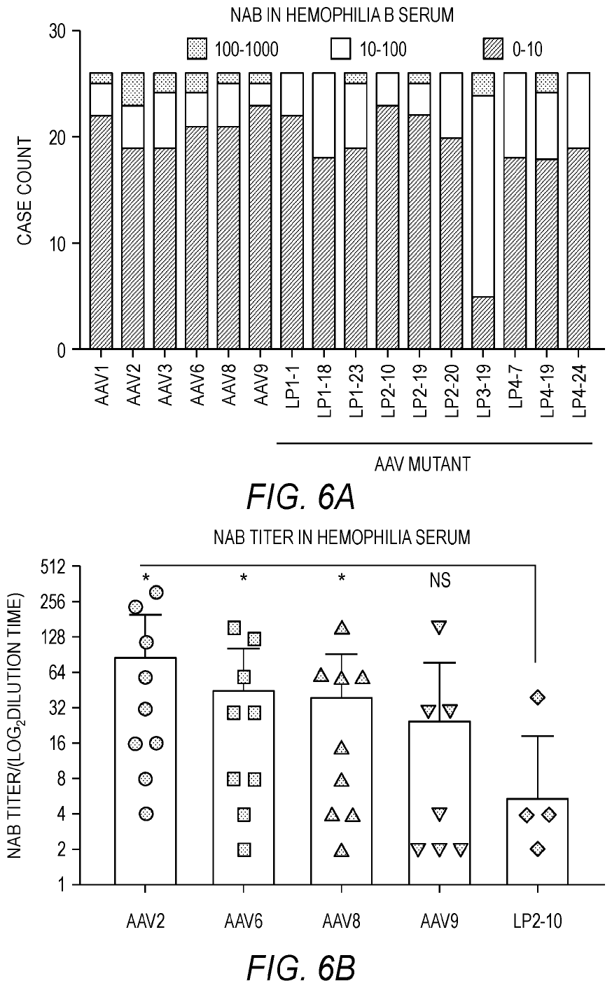

Based on the results from Nab analysis, mutant AAV LP2-10 demonstrated the ability to resist neutralization not only from IVIG but also from sera of individual human subjects. Next, the transduction efficiency of AAV LP2-10 was examined in human hepatocytes in vivo. As VP3 of AAV LP2-10 was composed of capsid from AAV6 and AAV8, and previous studies have demonstrated that AAV8 had higher human hepatocyte tropism than AAV6 in mice xenografted with human hepatocytes (Shao et al., Gene Ther. 26:504 (2019)), therefore, AAV8 was included as a control for comparison. Self-complementary AAV vectors encoding GFP at a dose of 1×10¹¹ particles were injected into humanized mice via retro-orbital vein. At week 3 post AAV injection, the livers from the mice were collected. Human hepatocyte transduction was assessed by immunohistochemistry staining (FIG. 6A) and flow cytometry (Table 5). Although mutant AAV LP2-10 showed high ability to evade Nabs as described above, the human hepatocyte transduction was not improved when compared to AAV8 in xenograft mice. Actually, AAV LP2-10 induced a little lower human hepatocytes transduction than AAV8 or at most similar to AAV8 in two separate experiments in mice xenografted from human hepatocytes of two different donors.

Previous studies from this laboratory have demonstrated that AAV8 had a relative high human hepatocyte tropism in xenografted mice when compared to other serotypes except from AAV7 (Shao et al., Gene Ther. 26:504 (2019)). In this study, several AAV mutants were successfully isolated from the livers of humanized mice in the presence of IVIG using a directed evolution approach with an AAV shuffled library. The majority of the mutants (9/10) had a C-terminus derived from AAV8, which may support the high human hepatocyte transduction of AAV8. Several mutants demonstrated the high ability to evade Nabs from IVIG and sera of healthy subjects and hemophilia patients when compared to AAV serotypes. Especially, the mutant LP2-10, the popular isolate in which the majority of VP3 subunit is derived from AAV8, had the highest Nab escape capacity to IVIG than any other mutants and serotypes. Human hepatocyte transduction with LP2-10 was similar to or a little lower than that with AAV8.

Overcoming Nab activity is the major challenge for AAV gene therapy in clinical trials, especially for patients who need systemic administration of AAV vectors. Approximately 50% of the human population possesses Nabs and cross-reactivity of AAV Nabs exists among serotypes. The existence of Nabs prevents patients from benefiting from effective AAV mediated gene therapy. Several approaches have been explored to evade AAV Nabs including clinical methods and lab approaches. Utilization of B cell depletion (Velazquez et al., Mol. Ther. Methods Clin. Dev. 4:159 (2017); Corti et al., Mol. Ther. Methods Clin. Dev. 1: 14033-. doi: 10.1038/mtm.2014.33 (2014)), plasma-apheresis (Monteilhet et al., Mol. Ther. 19:2084 (2011)), empty particles as a decoy (Manno et al., Nature Med. 12:342 (2006)) has been used in clinical trial, but these methods require either long-term administration or multiple cycles or competition for AAV transduction/increase of AAV capsid antigen load. Additionally, these methods show low efficacy and cause unwanted side effects or complications. More attention has been paid on genetic engineering of AAV capsid to evade AAV Nabs, including rational design and directed evolution. Rational design needs detailed information of AAV epitopes and the knowledge of AAV three dimension structure. However, directed evolution does not need this information and knowledge. Most isolated AAV mutants with Nab evasion were selected either from animal models in the absence of AAV Nabs or from cell lines in vitro in the presence of Nabs. These mutants were tested for their transduction in animal models. It is well known that the result generated from mice is not necessarily translatable to larger animals and human clinical trials with AAV vector gene therapy. For example, in hemophilia B AAV gene therapy, over 100 fold lower FIX expression was detected in patients than that in a mouse model when a similar dose of AAV vector is used (George et al., New Engl. J. Med. 377:2215 (2017)). Therefore, it is imperative to use authentic animal models to isolate AAV mutants in the presence of Nabs for human clinical applications. The mutants isolated from these systems may have human tissue specific tropism and the ability to resist Nabs. Humanized mice have been used to test AAV transduction efficiency and isolate AAV variants with specific tissue tropism. Especially, mice xenografted with human hepatocytes have been widely used to develop novel AAV mutants for high transduction efficiency. Some mutants have been isolated from the humanized mice and showed high human hepatocyte transduction and substantial high ability to evade Nabs when compared to parental serotypes using AAV shuffling library (Paulk et al., Mol. Ther. 26:289 (2018)). In this study, several AAV variants were successfully isolated from the liver of chimeric mice xenografted with human hepatocytes in the presence of human Nabs. Some of these mutants had a better IVIG evasion ability than AAV8, however, all mutants, except for the mutant AAV LP2-10, did not show a higher capacity for IVIG evasion when compared to AAV9. AAV9 has the most potential to resist Nab activity from IVIG among serotypes. It is interesting to note that the popular mutant AAVLP2-10 needs 4 more fold of IVIG than AAV9 for neutralization. This result strongly suggests that it is possible to isolate AAV mutants with higher potential to evade Nabs than parental AAV serotypes using directed evolution strategy with an AAV shuffled library. However, when the human samples from healthy subjects or hemophilia patients were tested, the profile of Nab escape for isolated mutants is different among individuals. Mutants including AAV/LP2-10 did not show Nab evasion potential in most samples equivalent to AAV9. This is consistent to our previous findings, AAV mutants isolated from muscles in the presence of patient serum had a better ability to escape the Nab from cognate serum (Li et al., Mol. Ther 24:53 (2016)). This consistent result may provide insight about the limitation of the selection of AAV mutants for Nabs evasion using directed evolution approach in the presence of Nabs.

Based on the interaction of monoclonal antibody with AAV virion, it has been identified that residue 265 on AAV2 can be recognized by A20 antibody, which is able to bind to intact AAV2 virions and block AAV2 transduction (Lochrie et al., J Virol. 80:821 (2006); Li et al., J. Virol. 86:7752 (2012)). The inventors' previous studies have demonstrated that modification of residue 265 demonstrated the decreased ability to bind to A20 and resisted A20 neutralizing activity (Li et al., J Virol. 86:7752 (2012)). In support of this finding, in this study, the domain covering residue 265 in most mutants was swapped from other serotypes although they had a C-terminus from AAV8. For example, the mutant LP2-10 had a domain from aa 261-272 of AAV6, which may suggest that the domain replacement with AAV6 which involves residue 265 may offer high resistance of mutant AAV LP2-10 to IVIG when compared to AAV8 and AAV6.

The point mutation E531K on the AAV8 C-terminus may also play a role to help the mutants resist neutralizing antibodies (Bartel et al., Gene Ther. 19:694 (2012)). Among 14 AAV mutants, only 5 mutants, including AAV LP3-19 and AAV LP4-19, did not have an E531K point mutation. Comparing to AAV8, it was found that a similar neutralizing antibody profile was observed in both mutants AAV LP3-19 and AAV LP4-19 in sera from humans, especially in the sera of hemophilia B patients. According to previous reports, E531K is responsible for the heparin binding ability of AAV6, which might have influenced the virus titer, transduction efficiency, and penetration of the blood brain barrier (Boye et al., J. Virol. 90:4215 (2016)). Additionally, the E531K mutation in AAV capsids could also affect neutralizing antibody resistance due to E531K being located in a basic cluster near the spikes that surround the icosahedral threefold axes of the AAV capsid (Li et al., J. Virol. 86:7752 (2012)). Several novel mutations were also identified on AAV8 capsid in isolated mutants including E417K, H51ON, T526S, R725H and N735P. Most of the AAV mutants isolated from this study contained a high viable composition of N-terminal sequence of the capsid from different serotypes. The novel mutations and chimeric N-terminus may play a role in these mutants in Nab resistance.

Similar to the previous finding from this laboratory that mutants isolated from mouse muscle in the presence of Nabs did not induce higher transduction than AAV6¹², the best serotype for mouse muscle tropism, the mutant AAV LP2-10 induced a similar or lower human hepatocyte transduction than AAV8 in a xenograft mouse model. Again, the low transduction of human hepatocyte from mutant AAV LP2-10 may result from substitution of the AAV8 domain from AAV6 on VP3 subunits. Previous studies have demonstrated that modification of residue 265 of VP1 impacts AAV transduction (Bowles et al., Mol. Ther. 20:443 (2012)), and a recent study showed that swapping the 265 domain from AAVrh10, which crosses the blood brain barrier in mice, into AAV1 enabled AAV1 to transport across the BBB (Albright et al., Mol. Ther. 26:510 (2018)). These studies demonstrated that the domain involving residue 265 plays a role in AAV fundamental function in addition to Nab recognition. It has been demonstrated that AAV6 induces much lower human hepatocyte transduction than AAV8 in chimeric humanized mice (Shao et al., Gene Ther. 26:504 (2019)). In combination with this observation and the low transduction of mutant AAV LP2-10 in human hepatocytes, it may be concluded that the domain around residue 265 may confer high AAV8 human hepatocyte tropism.

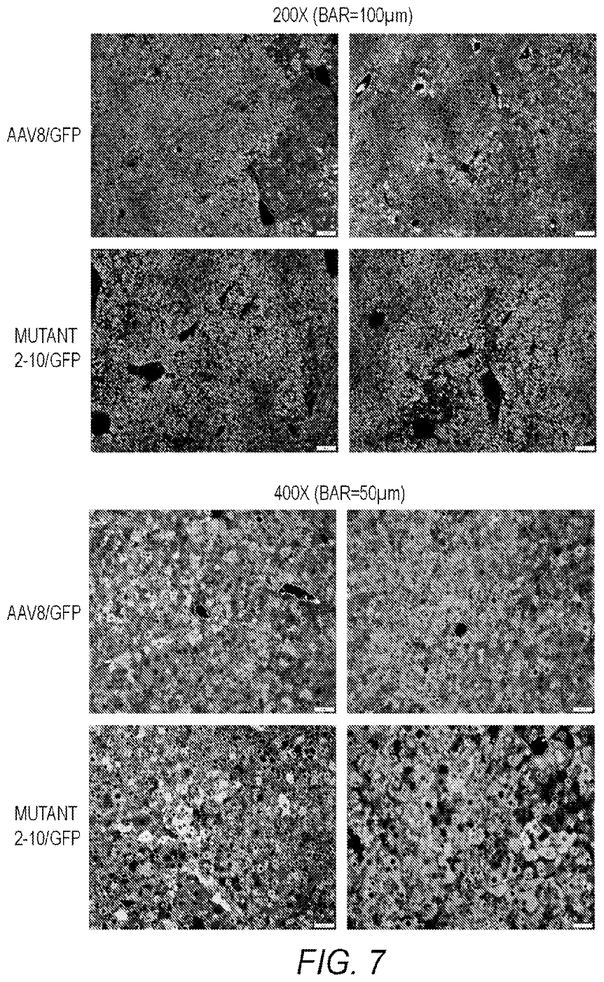

Structural studies have demonstrated the role of different domains on the AAV surface in AAV transduction efficiency and tissue tropism. These domains are also the sites for Nab recognition. The homology between wild-type AAV capsid sequences and the modified capsid sequences is shown in Table 6. A phylogenetic analysis was performed of the AAV mutants isolated from the liver of humanized mice and their parental serotypes, and it was found that most mutants were located in the same clade as AAV8. It is interesting to note that the most popular and effective Nab evasion mutant, AAV LP2-10, is more phylogenetically close to AAV8 when compared to other mutants (FIG. 7A). Finally, structural modeling of mutant AAV LP2-10 was performed to determine the relationship of structure-function (FIGS. 7B-7C). The domain of mutant AAV LP2-10 varied from the AAV8 capsid sequence between aa residues 261 and 272 which is derived from AAV1/6. This domain is covered by variable region (VR) I of AAV virions. It has been implicated that VRI is involved in AAV transduction and Nab recognition (Govindasamy et al., J. Virol. 80:11556 (2006)). The results from this study support that VRI on the AAV8 capsid plays a role in high human hepatocyte transduction and Nab recognition, and genetic modification of VRI may change transduction efficiency and evade Nabs.

In summary, the inventors were able to isolate AAV mutants with the ability to evade AAV neutralizing antibodies in humanized mice in the presence of Nabs, their transduction is comparable to the best AAV serotypes.

The foregoing is illustrative of the present invention, and is not to be construed as limiting thereof. The invention is defined by the following claims, with equivalents of the claims to be included therein.

TABLE 3
Nab titer in serum from normal human subjects

| | | | | | | | | LP1- | LP1- | LP2- | LP2- | LP2- | LP3- | LP4- | LP4- | LP4- |
|---|---|---|---|---|---|---|---|---|---|---|---|---|---|---|---|---|
| | AAV1 | AAV2 | AAV3 | AAV6 | AAV8 | AAV9 | LP1-1 | 18 | 23 | 10 | 19 | 20 | 19 | 7 | 19 | 24 |
| S1 | 100 | 800 | 400 | 50 | 100 | 50 | 100 | 200 | 200 | 25 | 200 | 800 | 100 | 100 | 100 | 25 |
| S2 | 250 | 1000 | 500 | 100 | 250 | 125 | 150 | 500 | 250 | 125 | 500 | 500 | 250 | 250 | 250 | 250 |
| S3 | 4 | 320 | 80 | 1 | 2 | 2 | 4 | 1 | 40 | 2 | 40 | 80 | 10 | 4 | 2 | 2 |
| S4 | 20 | 40 | 20 | 2 | 8 | 2 | 10 | — | 10 | 2 | 20 | 40 | 10 | 10 | 10 | 8 |
| S5 | — | 1 | — | — | — | — | — | — | — | — | — | 1 | — | — | — | — |
| S6 | — | — | — | — | — | — | — | — | — | — | — | — | — | — | — | — |
| S7 | 20 | 160 | 160 | 40 | 80 | 40 | 40 | 80 | 80 | 10 | 80 | 320 | 80 | 40 | 40 | 40 |
| S8 | — | 2 | 1 | — | — | — | — | 4 | — | — | — | 2 | — | — | 4 | — |
| S9 | 2 | 32 | 16 | 8 | 8 | — | 8 | 8 | 16 | 2 | 8 | 64 | 32 | 8 | 16 | 8 |
| S10 | 80 | 160 | 160 | 160 | 160 | 40 | 160 | 80 | 320 | 80 | 160 | 320 | 80 | 320 | 160 | 160 |
| S11 | 8 | 16 | 32 | 32 | 16 | 2 | 8 | 16 | 32 | 4 | 16 | 32 | 128 | 32 | 32 | 8 |
| S12 | — | 1 | 1 | — | 1 | — | 1 | 1 | 2 | — | — | — | — | 2 | 2 | — |
| S13 | 320 | 160 | 320 | 160 | 160 | 160 | 302 | 160 | 320 | 80 | 320 | 320 | 320 | 320 | 160 | 160 |
| S14 | 4 | 8 | 8 | 16 | 8 | 2 | 8 | 4 | 8 | 2 | 8 | 8 | 32 | 16 | 32 | 8 |
| S15 | 2 | — | 2 | 1 | 1 | — | 4 | 2 | 2 | 2 | — | 1 | 4 | 1 | 1 | 1 |
| S16 | — | — | — | 1 | — | — | 1 | 1 | 1 | 1 | 1 | — | 1 | 1 | 1 | — |
| S17 | — | — | 1 | 1 | — | — | — | 1 | — | — | — | — | — | — | 1 | — |
| S18 | — | 2 | 2 | 8 | 8 | — | 2 | 8 | 8 | 2 | 2 | 4 | >16 | 1 | 8 | — |
| S19 | 4 | 32 | 32 | 64 | 16 | — | 8 | 64 | 64 | 4 | 32 | 64 | 128 | 16 | 128 | 32 |
| S20 | 4 | 4 | 8 | 32 | 32 | 2 | 8 | 8 | 32 | 2 | 8 | 8 | 64 | 32 | 32 | 4 |

TABLE 4
Nab titer in serum from hemophilia patients

| | | | | | | | | LP1- | LP1- | LP2- | LP2- | LP2- | LP3- | LP4- | LP4- | LP4- |
|---|---|---|---|---|---|---|---|---|---|---|---|---|---|---|---|---|
| | AAV1 | AAV2 | AAV3 | AAV6 | AAV8 | AAV9 | LP1-1 | 18 | 23 | 10 | 19 | 20 | 19 | 7 | 19 | 24 |
| P1 | — | 2 | — | — | 2 | — | 2 | 2 | 2 | — | 2 | 2 | 8 | — | 2 | — |
| P2 | 60 | 120 | 60 | 30 | 60 | 30 | 60 | 60 | 60 | — | 60 | 60 | 60 | 60 | 30 | 30 |
| P3 | 4 | 16 | 16 | 2 | 4 | 2 | 32 | 64 | 64 | 32 | 16 | 16 | 32 | 32 | 128 | 32 |
| P4 | — | — | — | 2 | 2 | — | — | 2 | 2 | 2 | 4 | 4 | 8 | 2 | — | — |
| P5 | — | 2 | 2 | — | 4 | — | — | 2 | 4 | 2 | 2 | 2 | 16 | 2 | 2 | — |
| P6 | — | — | — | 2 | — | — | 2 | — | — | — | — | 2 | 16 | 2 | — | — |
| P7 | — | — | — | — | 2 | — | — | 2 | 2 | — | 2 | 2 | 32 | — | 4 | — |
| P8 | — | — | — | — | 2 | 2 | — | — | 2 | — | 4 | — | 64 | 2 | 2 | — |
| P9 | — | — | 2 | — | — | — | — | 64 | 2 | — | 2 | — | 32 | — | 2 | 2 |
| P10 | — | 16 | 16 | 2 | 2 | — | — | 64 | 32 | — | 8 | 4 | 32 | 64 | 16 | 8 |
| P11 | 30 | 240 | 120 | 30 | 15 | — | 15 | 60 | 60 | — | — | 30 | 60 | 60 | 60 | 30 |
| P12 | — | 4 | 2 | — | 2 | — | 4 | 2 | 2 | 2 | 2 | 2 | 4 | 8 | 8 | 4 |
| P22 | — | 4 | 4 | — | 2 | 2 | 4 | 2 | — | 2 | 2 | 2 | 32 | 4 | 4 | 8 |
| P23 | — | 2 | 2 | 2 | 2 | — | 2 | 2 | 2 | — | 2 | 2 | 64 | 4 | 2 | 2 |
| P26 | — | — | — | — | 2 | 2 | — | — | 2 | — | — | 2 | 64 | — | 2 | — |
| P27 | — | 2 | — | — | 2 | — | — | 2 | 2 | — | — | — | 8 | — | 16 | — |
| P28 | 8 | 32 | 32 | 8 | 8 | 2 | 8 | 2 | 4 | — | 8 | 16 | 8 | 8 | 4 | 4 |
| P29 | 4 | 4 | 4 | 128 | 64 | 4 | 4 | 64 | 128 | 32 | 2 | 8 | 128 | 32 | 128 | 64 |
| P30 | — | 2 | 2 | — | 2 | 2 | 2 | 2 | 8 | — | 8 | 2 | 32 | — | 2 | — |
| P31 | 4 | 8 | 8 | 4 | 4 | 2 | 4 | 4 | 8 | 4 | 4 | 2 | 32 | 16 | 4 | 16 |
| P32 | — | 2 | 2 | 2 | 2 | 2 | 2 | 2 | — | — | 8 | — | 32 | 8 | 2 | — |
| P33 | — | 2 | 2 | 2 | 2 | 2 | 4 | 2 | 2 | — | 2 | — | 32 | 2 | 2 | — |
| P34 | — | — | — | — | 2 | 2 | — | 2 | — | — | — | 2 | 32 | 2 | 2 | — |
| P35 | 320 | 320 | 320 | 320 | 160 | 160 | 80 | 80 | 80 | 40 | 160 | 80 | 160 | 80 | 80 | 40 |
| P36 | 30 | 60 | 60 | 30 | 60 | 30 | — | 60 | 30 | — | 30 | 15 | 60 | 60 | 60 | 15 |
| P37 | — | 2 | 2 | — | 2 | — | — | 2 | 2 | — | 2 | 2 | 16 | 2 | 2 | — |

TABLE 5
Human hepatocyte transduction with
mutant AAV LP2-10 in xenograft mice

| Donor | Mouse | | IHC Method | Flow Method | |
|---|---|---|---|---|---|
| # | ID | AAV | Transduction (%) | Transduction (%) | MFI |
| 1 | 2905 | 8 | 9.7 ± 2.7 | n.d. | n.d. |
| 1 | 2906 | 8 | 7.7 ± 2.7 | n.d. | n.d. |
| 1 | 2911 | LP2-10 | 4.5 ± 2.5 | n.d. | n.d. |
| 1 | 2912 | LP2-10 | 6.6 ± 5.4 | n.d. | n.d. |
| 2 | 39864 | 8 | 7.4 ± 2.7 | 15.6 | 26.1 |
| 2 | 39976 | 8 | 7.5 ± 2.0 | 4.5 | 27.1 |
| 2 | 39888 | LP2-10 | 5.0 ± 2.0 | 5.8 | 27.4 |
| 2 | 39893 | LP2-10 | 4.5 ± 2.0 | 6.4 | 41.8 |
| 2 | 39897 | LP2-10 | 4.0 ± 2.1 | 4.7 | 22.8 |

TABLE 6
Homology analysis of wild-type and modified capsid protein sequences

| | AAV1 | AAV2 | AAV3 | AAV4 | AAV5 | AAV6 | AAV7 | AAV8 | AAV9 | AAV10 | AAV11 | LP1-1 | LP1-11 |
|---|---|---|---|---|---|---|---|---|---|---|---|---|---|
| AAV1 | 100 | 83.29 | 86.7 | 63 | 57.95 | 99.18 | 85.09 | 83.88 | 82.63 | 84.69 | 66.26 | 85.6 | 83.7 |
| AAV2 | | 100 | 87.91 | 59.95 | 57.55 | 83.42 | 82.38 | 82.79 | 81.68 | 83.74 | 62.72 | 82.74 | 84.65 |
| AAV3 | | | 100 | 62.19 | 58.33 | 86.84 | 84.82 | 85.66 | 83.6 | 85.79 | 64.52 | 85.89 | 85.35 |
| AAV4 | | | | 100 | 52.62 | 62.87 | 63.09 | 63.32 | 62.33 | 63.45 | 81.22 | 62.95 | 61.48 |
| AAV5 | | | | | 100 | 58.09 | 57.93 | 57.93 | 56.8 | 57.2 | 53.04 | 57.55 | 56.74 |
| AAV6 | | | | | | 100 | 85.23 | 84.01 | 82.23 | 84.82 | 66.13 | 86.28 | 84.1 |
| AAV7 | | | | | | | 100 | 87.96 | 81.73 | 88.36 | 66.53 | 86.04 | 84.55 |
| AAV8 | | | | | | | | 100 | 85.23 | 93.36 | 65.01 | 95.66 | 94.17 |
| AAV9 | | | | | | | | | 100 | 85.77 | 63.27 | 86.84 | 86.7 |
| AAV10 | | | | | | | | | | 100 | 65.95 | 90.51 | 89.3 |
| AAV11 | | | | | | | | | | | 100 | 64.92 | 63.17 |
| LP1-1 | | | | | | | | | | | | 100 | 96.74 |
| LP1-11 | | | | | | | | | | | | | 100 |
| LP1-13 | | | | | | | | | | | | | |
| LP1-18 | | | | | | | | | | | | | |
| LP1-23 | | | | | | | | | | | | | |
| LP2-10 | | | | | | | | | | | | | |
| LP2-19 | | | | | | | | | | | | | |
| LP2-20 | | | | | | | | | | | | | |
| LP3-19 | | | | | | | | | | | | | |
| LP4-7 | | | | | | | | | | | | | |
| LP4-15 | | | | | | | | | | | | | |
| LP4-19 | | | | | | | | | | | | | |
| LP4-21 | | | | | | | | | | | | | |
| LP4-24 | | | | | | | | | | | | | |
| | | LP1-13 | LP1-18 | LP1-23 | LP2-10 | LP2-19 | LP2-20 | LP3-19 | LP4-7 | LP4-15 | LP4-19 | LP4-21 | LP4-24 |
| | AAV1 | 82.36 | 86.55 | 84.65 | 84.38 | 83.42 | 83.29 | 84.65 | 84.94 | 84.78 | 86.96 | 85.19 | 83.7 |
| | AAV2 | 85.21 | 82.74 | 82.2 | 83.02 | 85.19 | 99.86 | 86.12 | 82.63 | 97.96 | 82.88 | 84.51 | 84.78 |
| | AAV3 | 84.28 | 86.16 | 85.75 | 85.89 | 85.07 | 87.77 | 85.75 | 85.23 | 88.45 | 86.3 | 85.21 | 84.94 |
| | AAV4 | 61.8 | 63.27 | 62.68 | 62.82 | 61.34 | 60.08 | 61.34 | 62.12 | 61.29 | 63.27 | 61.8 | 61.74 |
| | AAV5 | 56.8 | 57.68 | 57.22 | 57.49 | 57.09 | 57.55 | 57.28 | 56.8 | 57.95 | 57.82 | 57.09 | 56.82 |
| | AAV6 | 82.5 | 86.96 | 85.05 | 84.78 | 83.83 | 83.42 | 84.92 | 85.35 | 84.92 | 87.09 | 85.87 | 84.38 |
| | AAV7 | 84.17 | 86.31 | 85.09 | 85.64 | 83.6 | 82.38 | 84.82 | 85.39 | 83.88 | 86.45 | 84.82 | 84.42 |
| | AAV8 | 95.12 | 96.48 | 94.99 | 96.61 | 93.9 | 82.93 | 94.17 | 95.93 | 95.93 | 96.34 | 94.44 | 94.17 |
| | AAV9 | 84.94 | 86.16 | 87.79 | 85.62 | 85.62 | 81.82 | 84.26 | 86.3 | 82.9 | 85.75 | 84.67 | 86.16 |
| | AAV10 | 89.16 | 90.65 | 90.11 | 91.06 | 87.8 | 83.74 | 88.75 | 90.51 | 85.23 | 90.51 | 88.75 | 88.89 |
| | AAV11 | 62.68 | 65.64 | 65.1 | 64.52 | 62.95 | 62.72 | 64.03 | 64.16 | 64.2 | 65.32 | 63.89 | 63.17 |
| | LP1-1 | 94.98 | 97.96 | 98.23 | 97.01 | 96.33 | 82.88 | 94.97 | 96.88 | 84.24 | 97.83 | 96.88 | 97.83 |
| | LP1-11 | 95.52 | 95.92 | 97.55 | 96.47 | 97.55 | 84.92 | 96.88 | 94.57 | 83.29 | 95.52 | 97.42 | 98.37 |
| | LP1-13 | 100 | 94.84 | 93.89 | 94.84 | 97.56 | 85.35 | 97.01 | 96.74 | 94.98 | 94.98 | 96.34 | 96.61 |
| | LP1-18 | | 100 | 96.74 | 96.88 | 95.65 | 83.02 | 96.74 | 97.29 | 84.65 | 99.32 | 97.69 | 96.47 |
| | LP1-23 | | | 100 | 97.28 | 95.92 | 82.47 | 94.43 | 96.07 | 84.1 | 96.6 | 95.24 | 96.2 |
| | LP2-10 | | | | 100 | 96.06 | 83.15 | 94.7 | 95.66 | 84.1 | 96.74 | 95.92 | 95.65 |
| | LP2-19 | | | | | 100 | 85.33 | 97.28 | 95.79 | 83.42 | 95.52 | 97.42 | 97.96 |
| | LP2-20 | | | | | | 100 | 86.26 | 82.77 | 98.1 | 83.15 | 84.78 | 85.05 |
| | LP3-19 | | | | | | | 100 | 95.79 | 84.35 | 96.88 | 98.23 | 97.01 |
| | LP4-7 | | | | | | | | 100 | 84.26 | 97.15 | 95.93 | 95.25 |
| | LP4-15 | | | | | | | | | 100 | 84.65 | 83.15 | 83.42 |
| | LP4-19 | | | | | | | | | | 100 | 97.55 | 96.06 |
| | LP4-21 | | | | | | | | | | | 100 | 98.23 |
| | LP4-24 | | | | | | | | | | | | 100 |

Sequences
LP1-1 nucleotide sequence
(SEQ ID NO: 1)
ATGGCTGCCGATGGTTATCTTCCAGATTGGCTCGAGGACAACCTCTCTGAGGGCATTCGCGAGTGGT
GGGACTTGAAACCTGGAGCCCCGAAACCCAAAGCCAACCAGCAAAAGCAGGACGACGGCCGGGGTCT
GGTGCTTCCTGGCTACAAGTACCTCGGACCCTTCAACGGACTCGACAAGGGGGAGCCCGTCAACGCG
GCGGATGCAGCGGCCCTCGAGCACGACAAGGCCTACGACCAGCAGCTCAAAGCGGGTGACAATCCGT
ACCTGCGGTATAACCACGCCGACGCCGAGTTTCAGGAGCGTCTGCAAGAAGATACGTCTTTTGGGGG
CAACCTCGGGCGAGCAGTCTTCCAGGCCAAGAAGAGGGTTCTCGAACCTTTTGGTCTGGTTGAGGAA
GGTGCTAAGACGGCTCCTGGAAAGAAACGTCCGGTAGAGCAGTCGCCACAAGAGCCAGACTCCTCCT
CGGGCATTGGCAAGACAGGCCAGCAGCCCGCTAAAAAGAGACTCAATTTTGGTCAGACTGGCGACAC
AGAGTCAGTCCCAGACCCTCAACCAATCGGAGAACCTCCCGCAGCCCCCTCAGGTGTGGGATCTCTT
ACAATGGCTTCAGGTGGTGGCGCACCAGTGGCAGACAATAACGAAGGTGCCGATGGAGTGGGTAGTT
CCTCGGGAAATTGGCATTGCGATTCCCAATGGCTGGGCGACAGAGTCATCACCACCAGCACCCGAAC
CTGGGCCCTGCCCACTTACAACAACCATCTCTACAAGCAAATCTCCAGTGCTTCAACGGGGGCCAGC
AACGACAACCACTACTTCGGCTACAGCACCCCCTGGGGGTATTTTGACTTCAACAGATTCCACTGCC
ACTTCTCACCACGTGACTGGCAGCGACTCATCAACAACAACTGGGGATTCCGGCCTAAGCGACTCAA
CTTCAAACTCTTCAACATTCAGGTCAAAGAGGTTACGGACAACAATGGAGTCAAGACCATCGCCAAT
AACCTTACCAGCACGGTCCAGGTCTTCACGGACTCGGAGTACCAGTTGCCGTACGTCCTCGGCTCTG
CGCACCAGGGCTGCCTCCCTCCGTTCCCGGCGGACGTGTTCATGATTCCGCAGTACGGCTACCTAAC
GCTTAACAATGGCAGCCAGGCAGTGGGACGGTCATCCTTCTACTGCCTGGAATACTTTCCTTCGCAG
ATGCTGAGAACCGGCAACAACTTCCAGTTTACTTACACCTTCGAGGACGTGCCTTTCCACAGCAGCT
ACGCCCACAGCCAGAGCTTGGACCGGCTGATGAATCCTCTGATTGACCAGTACCTGTACTACTTGTC
TCGGACTCAAACAACAGGAGGCACGGCAAATACGCAGACTCTGGGCTTCAGCCAAGGTGGGCCTAAT
ACAATGGCCAATCAGGCAAAGAACTGGCTGCCAGGACCCTGTTACCGCCAACAACGCGTCTCAACGA
CAACCGGGCAAAACAACAATAGCAACTTTGCCTGGACTGCTGGGACCAAATACCATCTGAATGGAAG
AAATTCATTGGCTAATCCTGGCATCGCTATGGCCTCACACAAAGACGACAAGGAGCGTTTTTTTCCC
AGTAACGGGATCCTGATTTTTGGCAAACAAAATGCTGCCAGAGACAATGCGGATTACAGCGATGTCA
TGCTCACCAGCGAGGAAGAAATCAAAACCACTAACCCTGTGGCTACAGAGGAATACGGTATCGTGGC
AGATAACTTGCAGCAGCAAAACACGGCTCCTCAAATTGGAACTGTCAACAGCCAGGGGGCCTTACCC
GGTATGGTCTGGCAGAACCGGGACGTGTACCTGCAGGGTCCCATCTGGGCCAAGATTCCTCACACGG
ACGGCAACTTCCACCCGTCTCCGCTGATGGGCGGCTTTGGCCTGAAACATCCTCCGCCTCAGATCCT
GATCAAGAACACGCCTGTACCTGCGGATCCTCCGACCACCTTCAACCAGTCAAAGCTGAACTCTTTC
ATCACGCAATACAGCACCGGACAGGTCAGCGTGGAAATTGAATGGGAGCTGCAGAAGGAAAACAGCA
AGCGCTGGAACCCCGAGATCCAGTACACCTCCAACTACTACAAATCTACAAGTGTGGACTTTGCTGT
TAATACAGAAGGCGTGTACTCTGAACCCCACCCCATTGGCACCCGTTACCTCACCCGTCCCCTGTAA
LP1-11 nucleotide sequence
(SEQ ID NO: 2)
ATGGCTGCCGATGGTTATCTTCCAGATTGGCTCGAGGACACTCTCTCTGAAGGAATAAGACAGTGGT
GGAAGCTCAAACCTGGCCCACCACCACCAAAGCCCGCAGAGCGGCATAAGGACGACAGCAGGGGTCT
TGTGCTTCCTGGGTACAAGTACCTCGGACCCTTCAACGGACTCGACAAGGGAGAGCCGGTCAACGCG
GCGGACGCAGCGGCCCTCGAGCACGACAAGGCCTACGACCAGCAGCTGCAGGCGGGTGACAATCCGT
ACCTGCGGTATAACCACGCCGACGCCGAGTTTCAGGAGCGTCTGCAAGAAGATACGTCTTTTGGGGG
CAACCTCGGGCGAGCAGTCTTCCAGGCCAAAAAGAGGCTTCTTGAACCTCTTGGTCTGGTTGAGGAA
GCGGCTAAGACGGCTCCTGGAAAGAAGAGGCCTGTAGAGCAGTCTCCTCAGGAACCGGACTCCTCCG
CGGGTATTGGCAAATCGGGTGCACAGCCCGCTAAAAAGAGACTCAATTTCGGTCAGACTGGCGACAC
AGAGTCAGTCCCAGACCCTCAACCAATCGGAGAACCTCCCGCAGCCCCCTCAGGTGTGGGATCTCTT
ACAATGGCTTCAGGTGGTGGCGCACCAGTGGCAGACAATAACGAAGGCGCCGACGGAGTGGGTAGTT
CCTCAGGAAATTGGCATTGCGATTCCACATGGCTGGGCGACAGAGTCATCACCACCAGCACCAGAAC
CTGGGCCCTGCCCACCTACAACAACCACCTCTACAAGCAAATCTCCAGTGCTTCAACGGGGGCCAGC
AACGACAACCACTACTTCGGCTACAGCACCCCTTGGGGGTATTTTGACTTCAACAGATTCCACTGCC
ACTTTTCACCACGTGACTGGCAAAGACTCATCAACAACAACTGGGGATTCCGACCCAAGAGACTCAA
CTTCAAGCTCTTTAACATCCAGGTCAAAGAGGTTACGGACAACAATGGAGTCAAGACCATCGCCAAT
AACCTTACCAGCACGGTCCAGGTCTTCACGGACTCGGAGTACCAGTTGCCGTACGTCCTCGGCTCTG
CGCACCAGGGCTGCCTCCCTCCGTTCCCGGCGGACGTGTTCATGATTCCGCAGTACGGCTACCTAAC
GCTCAACAATGGCAGCCAGGCAGTGGGACGGTCATCCTTTTACTGCCTGGAATATTTCCCATCGCAG
ATGCTGAGAACGGGCAATAACTTTACCTTCAGCTACACCTTCGAGGACGTGCCTTTCCACAGCAGCT
ACGCCCACAGCCAGAGCTTGGACCGGCTGATGAATCCTCTGATTGACCAGTACCTGTACTACTTGTC
TCGGACTCAAACAACAGGAGGCACGGCAAATACGCAGACTCTGGGCTTCAGCCAAGGTGGGCCTAAT
ACAATGGCCAATCAGGCAAAGAACTGGCTGCCAGGACCCTGTTACCGCCAACAACGCGTCTCAACGA
CAACCGGGCAAAACAACAATAGCAACTTTGCCTGGACTGCTGGGACCAAATACCATCTGAATGGAAG
AAATTCATTGGCTAATCCTGGCATCGCTATGGCCTCACACAAAGACGACAAGGAGCGTTTTTTTCCC
AGTAACGGGATCCTGATTTTTGGCAAACAAAATGCTGCCAGAGACAATGCGGATTACAGCGATGTCA
TGCTCACCAGCGAGGAAGAAATCAAAACCACTAACCCTGTGGCTACAGAGGAATACGGTATCGTGGC
AGATAACTTGCAGCAGCAAAACACGGCTCCTCAAATTGGAACTGTCAACAGCCAGGGGGCCTTACCC
GGTATGGTCTGGCAGAACCGGGACGTGTACCTGCAGGGTCCCATCTGGGCCAAGATTCCTCACACGG
ACGGCAACTTCCACCCGTCTCCGCTGATGGGCGGCTTTGGCCTGAAACATCCTCCGCCTCAGATCCT
GATCAAGAACACGCCTGTACCTGCGGATCCTCCGACCACCTTCAACCAGTCAAAGCTGAACTCTTTC
ATCACGCAATACAGCACCGGACAGGTCAGCGTGGAAATTGAATGGGAGCTGCAGAAGGAAAACAGCA
AGCGCTGGAACCCCGAGATCCAGTACACCTCCAACTACTACAAATCTACAAGTGTGGACTTTGCTGT
TAATACAGAAGGCGTGTACTCTGAACCCCGCCCCATTGGCACCCGTTACCTCACCCGTAATCTGTAA
LP1-13 nucleotide sequence
(SEQ ID NO: 3)
ATGGCTGCCGATGGTTATCTTCCAGATTGGCTCGAGGACACTCTCTCTGAAGGAATAAGACAGTGGT
GGAAGCTCAAACCTGGCCCACCACCACCAAAGCCCGCAGAGCGGCATAAGGACGACAGCAGGGGTCT
TGTGCTTCCTGGGTACAAGTACCTCGGACCCTTCAACGGACTCGACAAGGGAGAGCCGGTCAACGAG
GCAGACGCCGCGGCCCTCGAGCACGACAAAGCCTACGACCGGCAGCTCGACAGCGGAGACAACCCGT
ACCTCAAGTACAACCACGCCGACGCGGAGTTTCAGGAGCGCCTTAAAGAAGATACGTCTTTTGGGGG
CAACCTCGGACGAGCAGTCTTCCAGGCGAAAAAGAGGGTTCTTGAACCTCTGGGCCTGGTTGAGGAA
CCTGTTAAGACGGCTCCGGGAAAAAAGAGGCCGGTAGAGCAGTCGCCACAAGAGCCAGACTCCTCCT
CGGGCATCGGCAAGACAGGCCAGCAGCCCGCTAAAAAGAGACTCAATTTTGGTCAGACTGGCGACTC
AGAGTCAGTCCCCGACCCACAACCTCTCGGAGAACCTCCAGCAGCGCCCTCTGGTGTGGGACCTAAT
ACAATGGCTGCAGGCGGTGGCGCACCAATGGCAGACAATAACGAAGGTGCCGATGGAGTGGGTAGTT
CCTCGGGAAATTGGCATTGCGATTCCCAATGGCTGGGGGACAGAGTCATCACCACCAGCACCCGAAC
CTGGACCCTGCCCACCTACAACAATCACCTCTACAAGCAAATCTCCAACAGCACATCTGGAGGAGCC
ACCAACGACAACACCTACTTCGGCTACAGCACCCCCTGGGGGTATTTTGACTTCAACAGATTCCACT
GCCACTTCTCACCACGTGACTGGCAGCGACTCATCAACAACAACTGGGGATTCCGGCCTAAGCGACT
CAACTTCAAGCTCTTCAACATTCAGGTCAAAGAGGTTACGGACAACAATGGAGTCAAGACCATCGCC
AATAACCTTACCAGCACGGTCCAGGTCTTCACGGACTCGGAGTACCAGTTGCCGTACGTCCTCGGCT
CTGCGCACCAGGGCTGCCTCCCTCCGTTCCCGGCGGACGTGTTCATGATTCCGCAGTACGGCTACCT
AACGCTCAACAATGGCAGCCAGGCAGTGGGACGGTCATCCTTTTACTGCCTGGAATATTTCCCATCG
CAGATGCTGAGAACCGGCAACAACTTCCAGTTTACTTACACCTTCGAGGACGTGCCTTTCCACAGCA
GCTACGCGCACAGCCAGAGCTTGGACCGGCTGATGAATCCTCTGATTGACCAGTACCTGTACTACTT
GTCTCGGACTCAAACAACAGGAGGCACGGCAAATACGCAGACTCTGGGCTTCAGCCAAGGTGGGCCT
AATACAATGGCCAATCAGGCAAAGAACTGGCTGCCAGGACCCTGTTACCGCCAACAACGCGTCTCAA
CGACAACCGGGCAAAACAACAATAGCAACTTTGCCTGGACTGCTGGGACCAAATACCATCTGAATGG
AAGAAATTCATTGGCTAATCCTGGCATCGCTATGGCAACACACAAAGACGACGAGGAGCGTTTTTTT
CCCAGTAACGGGATCCTGATTTTTGGCAAACAAAATGCTGCCAGAGACAATGCGGATTACAGCGATG
TCATGCTCACCAGCGAGGAAGAAATCAAAACCACTAACCCTGTGGCTACAGAGGAATACGGTATCGT
GGCAGATAACTTGCAGCAGCAAAACACGGCTCCTCAAATTGGAACTGTCAACAGCCAGGGGGCCTTA
CCCGGTATGGTCTGGCAGAACCGGGACGTGTACCTGCAGGGTCCCATCTGGGCCAAGATTCCTCACA
CGGACGGCAACTTCCACCCGTCTCCGCTGATGGGCGGCTTTGGCCTGAAACATCCTCCGCCTCAGAT
CCTGATCAAGAACACGCCTGTACCTGCGGATCCTCCGACCACCTTCAACCAGTCAAAGCTGAACTCT
TTCATCACGCAATACAGCACCGGACAGGTCAGCGTGGAAATTGAATGGGAGCTGCAGAAGGAAAACA
GCAAGCGCTGGAACCCCGAGATCCAGTACACCTCCAACTACTACAAATCTACAAGTGTGGACTTTGC
TGTTAATACAGAAGGCGTGTACTCTGAACCCCACCCCATTGGCACCCGTTACCTCACCCGTCCCCTG
TAA
LP1-18 nucleotide sequence
(SEQ ID NO: 4)
ATGGCTGCCGATGGTTATCTTCCAGATTGGCTCGAGGACAACCTCTCTGAGGGCATTCGCGAGTGGT
GGGCGCTGAAACCTGGAGCCCCGAAGCCCAAAGCCAACCAGCAAAAGCAGGACGACGGCCGGGGTCT
GGTGCTTCCTGGCTACAAGTACCTCGGACCCTTCAACGGACTCGACAAGGGGGAGCCCGTCAACGCG
GCGGACGCAGCGGCCCTCGAGCACGACAAGGCCTACGACCAGCAGCTCAAAGCGGGTGACAATCCGT
ACCTGCGGTATAACCACGCCGACGCCGAGTTTCAGGAGCGTCTGCAAGAAGATACGTCTTTTGGGGG
CAACCTCGGGCGAGCAGTCTTCCAGGCCAAGAAGCGGGTTCTCGAACCTCTCGGTCTGGTTGAGGAA
GGCGCTAAGACGGCTCCTGGAAAGAAACGTCCGGTAGAGCAGTCGCCACAAGAGCCAGACTCCTCCT
CGGGCATCGGCAAGACAGGCCAGCAGCCCGCTAAAAAGAGACTCAATTTTGGTCAGACTGGCGACTC
AGAGTCAGTCCCCGACCCACAACCTCTCGGAGAACCTCCAGCAACCCCCGCTGCTGTGGGACCTACT
ACAATGGCTTCAGGCGGTGGCGCACCAATGGCAGACAATAACGAAGGCGCCGACGGAGTGGGTAGTT
CCTCGGGAAATTGGCATTGCGATTCCACATGGCTGGGCGACAGAGTCATCACCACCAGCACCCGAAC
ATGGGCCTTGCCCACCTATAACAACCACCTCTACAAGCAAATCTCCAGTGCTTCAACGGGGGCCAGC
AACGACAACCACTACTTCGGCTACAGCACCCCCTGGGGGTATTTTGACTTCAACAGATTCCACTGCC
ACTTCTCACCACGTGACTGGCAGCGACTCATCAACAACAACTGGGGATTCCGGCCTAAGCGACTCAA
CTTCAAGCTCTTCAACATTCAGGTCAAAGAGGTTACGGACAACAATGGAGTCAAGACCATCGCCAAT
AACCTTACCAGCACGGTCCAGGTCTTCACGGACTCGGAGTACCAGTTGCCGTACGTCCTCGGCTCTG
CGCACCAGGGCTGCCTCCCTCCGTTCCCGGCGGACGTGTTCATGATTCCTCAGTACGGGTATCTGAC
GCTTAATGATGGAAGCCAGGCCGTGGGACGCTCCTCCTTCTACTGCCTGGAATACTTTCCTTCGCAG
ATGCTGAGAACCGGCAACAACTTCCAGTTTACTTACACCTTCGAGGACGTGCCTTTCCACAGCAGCT
ACGCCCACAGCCAGAGCTTGGACCGGCTGATGAATCCTCTGATTGACCAGTACCTGTACTACTTGTC
TCGGACTCAAACAACAGGAGGCACGGCAAATACGCAGACTCTGGGCTTCAGCCAAGGTGGGCCTAAT
ACAATGGCCAATCAGGCAAAGAACTGGCTGCCAGGACCCTGTTACCGCCAACAACGCGTCTCAACGA
CAACCGGGCAAAACAACAATAGCAACTTTGCCTGGACTGCTGGGACCAAATACCATCTGAATGGAAG
AAATTCATTGGCTAATCCTGGCATCGCTATGGCAACACACAAAGACGACAAGGAGCGTTTTTTTCCC
AGTAACGGGATCCTGATTTTTGGCAAACAAAATGCTGCCAGAGACAATGCGGATTACAGCGATGTCA
TGCTCACCAGCGAGGAAGAAATCAAAACCACTAACCCTGTGGCTACAGAGGAATACGGTATCGTGGC
AGATAACTTGCAGCAGCAAAACACGGCTCCTCAAATTGGAACTGTCAACAGCCAGGGGGCCTTACCC
GGTATGGTCTGGCAGAACCGGGACGTGTACCTGCAGGGTCCCATCTGGGCCAAGATTCCTCACACGG
ACGGCAACTTCCACCCGTCTCCGCTGATGGGCGGCTTTGGCCTGAAACATCCTCCGCCTCAGATCCT
GATCAAGAACACGCCTGTACCTGCGGATCCTCCGACCACCTTCAACCAGTCAAAGCTGAACTCTTTC
ATCACGCAATACAGCACCGGACAGGTCAGCGTGGAAATTGAATGGGAGCTGCAGAAGGAAAACAGCA
AGCGCTGGAACCCCGAGATCCAGTACACCTCCAACTACTACAAATCTACAAGTGTGGACTTTGCTGT
TAATACAGAAGGCGTGTACTCTGAACCCCGCCCCATTGGCACCCGTTACCTCACCCGTAATCTGTAA
LP1-23 nucleotide sequence
(SEQ ID NO: 5)
ATGGCTGCCGATGGTTATCTTCCAGATTGGCTCGAGGACAACCTCTCTGAGGGCATTCGCGAGTGGT
GGGCGCTGAAACCTGGAGCCCCGAAGCCCAAAGCCAACCAGCAAAAGCAGGACGACGGCCGGGGTCT
GGTGCTTCCTGGCTACAAGTACCTCGGACCCTTCAACGGACTCGACAAGGGGGAGCCCGTCAACGCG
GCGGATGCAGCGGCCCTCGAGCACGACAAGGCCTACGACCAGCAGCTCCAAGCGGGTGACAATCCGT
ACCTGCGGTATAACCACGCCGACGCCGAGTTTCAGGAGCGTCTTCAAGAAGATACGTCTTTTGGGGG
CAACCTCGGGCGAGCAGTCTTCCAGGCCAAAAAGAGGCTTCTTGAACCTCTTGGTCTGGTTGAGGAA
GCGGCTAAGACGGCTCCTGGAAAGAAGAGGCCTGTAGAGCAGTCTCCTCAGGAACCGGACTCCTCCG
CGGGTATTGGCAAATCGGGTGCACAGCCCGCCAAAAAGAGACTCAATTTCGGTCAGACTGGCGGCAC
AGAGTCAGTTCCAGACCCTCAACCAATCGGAGAACCTCCCGCAGCCCCCTCAGGTGTGGGATCTCTT
ACAATGGCTTCAGGTGGTGGCGCACCAGTGGCAGACAATAACGAAGGTGCCGATGGAGTGGGTAGTT
CCTCGGGAAATTGGCATTGCGATTCCCAATGGCTGGGGGACAGAGTCATCACCACCAGCACCCGAAC
CTGGGCCCTGCCCACCTACAACAATCACCTCTACAAGCAAATCTCCAGTGCTTCAACGGGGGCCAGC
AACGACAACCACTACTTCGGCTACAGCACCCCCTGGGGGTATTTTGACTTCAACCGCTTCCACTGCC
ACTTCTCACCACGTGACTGGCAGCGACTCATCAACAACAACTGGGGATTCCGGCCTAAGCGACTCAA
CTTCAAGCTCTTCAACATTCAGGTCAAAGAGGTTACGGACAACAATGGAGTCAAGACCATCGCCAAT
AACCTTACCAGCACGGTCCAGGTCTTCACGGACTCGGACTATCAGCTCCCGTACGTGCTCGGGTCGG
CTCACGAGGGCTGCCTCCCTCCGTTCCCGGCGGACGTGTTCATGATTCCGCAGTACGGCTACCTAAC
GCTCAACAATGGCAGCCAGGCAGTGGGACGGTCATCCTTTTACTGCCTGGAATATTTCCCATCGCAG
ATGCTGAGAACGGGCAACAACTTTACCTTCAGCTACACCTTTGAGGACGTTCCTTTCCACAGCAGCT
ACGCTCACAGCCAGAGTCTGGACCGGCTGATGAATCCTCTGATTGACCAGTACCTGTACTACTTGTC
TCGGACTCAAACAACAGGAGGTACGGCAAATACGCAGACTCTGGGCTTCAGCCAAGGTGGGCCTAAT
ACAATGGCCAATCAGGCAAAGAACTGGCTGCCAGGACCCTGTTACCGCCAACAACGCGTCTCAACGA
CAACCGGGCAAAACAACAATAGCAACTTTGCCTGGACTGCTGGGACCAAATACCATCTGAATGGAAG
AAATTCATTGGCTAATCCTGGCATCGCTATGGCCTCACACAAAGACGACAAGGAGCGTTTTTTTCCC
AGTAACGGGATCCTGATTTTTGGCAAACAAAATGCTGCCAGAGACAATGCGGATTACAGCGATGTCA
TGCTCACCAGCGAGGAAGAAATCAAAACCACTAACCCTGTGGCTACAGAGGAATACGGTATCGTGGC
AGATAACTTGCAGCAGCAAAACACGGCTCCTCAAATTGGAACTGTCAACAGCCAGGGGGCCTTACCC
GGTATGGTCTGGCAGAACCGGGACGTGTACCTGCAGGGTCCCATCTGGGCCAAGATTCCTCACACGG
ACGGCAACTTCCACCCGTCTCCGCTGATGGGCGGCTTTGGCCTGAAACATCCTCCGCCTCAGATCCT
GATCAAGAACACGCCTGTACCTGCGGATCCTCCGACCACCTTCAACCAGTCAAAGCTGAACTCTTTC
ATCACGCAATACAGCACCGGACAGGTCAGCGTGGAAATTGAATGGGAGCTGCAGAAGGAAAACAGCA
AGCGCTGGAACCCCGAGATCCAGTACACCTCCAACTACTACAAATCTACAAGTGTGGACTTTGCTGT
TAATACAGAAGGCGTGTACTCTGAACCCCACCCCATTGGCACCCGTTACCTCACCCGTCCCCTGTAA
LP2-10 nucleotide sequence
(SEQ ID NO: 6)
ATGGCTGCCGATGGTTATCTTCCAGATTGGCTCGAGGACACTCTCTCTGAAGGAATAAGACAGTGGT
GGAAGCTCAAACCTGGAGCCCCGAAACCCAAAGCCAACCAGCAAAAGCAGGACGACGGCCGGGGTCT
GGTGCTTCCTGGCTACAAGTACCTCGGACCCTTCAACGGACTCGACAAGGGGGAGCCCGTCAACGCG
GCGGATGCAGCGGCCCTCGAGCACGACAAGGCCTACGACCAGCAGCTCAAAGCGGGTGACAATCCGT
ACCTGCGGTATAACCACGCCGACGCCGAGTTTCAGGAGCGTCTGCAAGAAGATACGTCTTTTGGGGG
CAACCTCGGGCGAGCAGTCTTCCAGGCCAAAAAGAGGCTTCTTGAACCTCTTGGTCTGGTTGAGGAA
GCGGCTAAGACGGCTCCTGGAAAGAAGAGGCCTGTAGAGCAGTCTCCTCAGGAACCGGACTCCTCCG
CGGGTATTGGCAAATCGGGTGCACAGCCCGCCAAAAAGAGACTCAATTTCGGTCAGACTGGCGGCAC
AGAGTCAGTTCCAGACCCTCAACCTCTCGGAGAACCTCCAGCAGCGCCCTCTGGTGTGGGACCTAAT
ACAATGGCTGCAGGCGGTGGCGCACCAATGGCAGACAATAACGAAGGCGCCGACGGAGTGGGTAGTT
CCTCGGGAAATTGGCATTGCGATTCCACATGGCTGGGCGACAGAGTCATCACCACCAGCACCCGAAC
CTGGGCCCTGCCCACCTACAACAACCACCTCTACAAGCAAATCTCCAGTGCTTCAACGGGGGCCAGC
AACGACAACCACTACTTCGGCTACAGCACCCCCTGGGGGTATTTTGACTTTAACAGATTCCACTGCC
ACTTTTCACCACGTGACTGGCAGCGACTCATCAACAACAACTGGGGATTCCGGCCCAAGAGACTCAG
CTTCAAGCTCTTCAACATCCAGGTCAAGGAGGTCACGCAGAATGAAGGCACCAAGACCATCGCCAAT
AACCTCACCAGCACCATCCAGGTGTTTACGGACTCGGAGTACCAGCTGCCGTACGTTCTCGGCTCCG
CCCACCAGGGCTGCCTGCCTCCGTTCCCGGCGGACGTCTTCATGATTCCTCAGTACGGGTACCTGAC
TCTGAACAATGGCAGTCAGGCCGTGGGACGCTCCTCCTTCTACTGCCTGGAATACTTTCCTTCGCAG
ATGCTGAGAACCGGCAACAACTTCCAGTTTACTTACACCTTCGAGGACGTGCCTTTCCACAGCAGCT
ACGCGCACAGCCAGAGCTTGGACCGGCTGATGAATCCTCTGATTGACCAGTACCTGTACTACTTGTC
TCGGACTCAAACAACAGGAGGCACGGCAAATACGCAGACTCTGGGCTTCAGCCAAGGTGGGCCTAAT
ACAATGGCCAATCAGGCAAAGAACTGGCTGCCAGGACCCTGTTACCGCCAACAACGCGTCTCAACGA
CAACCGGGCAAAACAACAATAGCAACTTTGCCTGGACTGCTGGGACCAAATACCATCTGAATGGAAG
AAATTCATTGGCTAATCCTGGCATCGCTATGGCAACACACAAAGACGACAAGGAGCGTTTTTTTCCC
AGTAACGGGATCCTGATTTTTGGCAAACAAAATGCTGCCAGAGACAATGCGGATTACAGCGATGTCA
TGCTCACCAGCGAGGAAGAAATCAAAACCACTAACCCTGTGGCTACAGAGGAATACGGTATCGTGGC
AGATAACTTGCAGCAGCAAAACACGGCTCCTCAAATTGGAACTGTCAACAGCCAGGGGGCCTTACCC
GGTATGGTCTGGCAGAACCGGGACGTGTACCTGCAGGGTCCCATCTGGGCCAAGATTCCTCACACGG
ACGGCAACTTCCACCCGTCTCCGCTGATGGGCGGCTTTGGCCTGAAACATCCTCCGCCTCAGATCCT
GATCAAGAACACGCCTGTACCTGCGGATCCTCCGACCACCTTCAACCAGTCAAAGCTGAACTCTTTC
ATCACGCAATACAGCACCGGACAGGTCAGCGTGGAAATTGAATGGGAGCTGCAGAAGGAAAACAGCA
AGCGCTGGAACCCCGAGATCCAGTACACCTCCAACTACTACAAATCTACTAGTGTGGACTTTGCTGT
TAATACAGAAGGCGTGTACTCTGAACCCCACCCCATTGGCACCCGTTACCTCACCCGTCCCCTGTAA
LP2-19 nucleotide sequence
(SEQ ID NO: 7)
ATGGCTGCCGATGGTTATCTTCCAGATTGGCTCGAGGACACTCTCTCTGAAGGAATAAGACAGTGGT
GGAAGCTCAAACCTGGCCCACCACCACCAAAGCCCGCAGAGCGGCATAAGGACGACAGCAGGGGTCT
TGTGCTTCCTGGGTACAAGTACCTCGGACCCTTCAACGGACTCGACAAGGGAGAGCCGGTCAACGAG
GCAGACGCCGCGGCCCTCGAGCACGACAAAGCCTACGACCGGCAGCTCGACAGCGGAGACAACCCGT
ACCTCAAGTACAACCACGCCGACGCGGAGTTTCAGGAGCGCCTTAAAGAAGATACGTCTTTTGGGGG
CAACCTCGGACGAGCAGTCTTCCAGGCCAAGAAGCGGGTTCTTGAACCTCTTGGTCTGGTTGAGGAA
GGCGCTAAGACGGCTCCTGGAAAGAAGAGGCCTGTAGAGCAGTCTCCTCAGGAACCGGACTCCTCCG
CGGGTATTGGCAAATCGGGTGCACAGCCCGCCAAAAAGAGACTCAATTTCGGTCAGACTGGCGACTC
AGAGTCAGTTCCAGACCCTCAACCTCTCGGAGAACCTCCAGCAGCGCCCTCTGGTGTGGGACCTAAT
ACAATGGCTTCAGGTGGTGGCGCACCAGTGGCAGACAATAACGAAGGTGCCGATGGAGTGGGTAGTT
CCTCGGGAAATTGGCATTGCGATTCCCAATGGCTGGGCGACAGAGTCATCACCACCAGCACCCGAAC
CTGGGCCCTGCCCACTTACAACAACCATCTCTACAAGCAAATCTCCAGTGCTTCAACGGGGGCCAGC
AACGACAACCACTACTTCGGCTACAGCACCCCCTGGGGGTATTTTGACTTCAACAGATTCCACTGCC
ACTTCTCACCACGTGACTGGCAGCGACTCATCAACAACAACTGGGGATTCCGGCCTAAGCGACTCAA
CTTCAAACTCTTCAACATTCAGGTCAAAGAGGTTACGGACAACAATGGAGTCAAGACCATCGCCAAT
AACCTTACCAGCACGGTCCAGGTCTTCACGGACTCGGAGTACCAGTTGCCGTACGTCCTCGGCTCTG
CGCACCAGGGCTGCCTCCCTCCGTTCCCGGCGGACGTGTTCATGATTCCTCAGTACGGCTACCTAAC
GCTCAACAATGGCAGCCAGGCAGTGGGACGGTCATCCTTTTACTGCCTGGAATATTTCCCATCGCAG
ATGCTGAGAACCGGCAACAACTTCCAGTTTACTTACACCTTCGAGGACGTGCCTTTCCACAGCAGCT
ACGCGCACAGCCAGAGCTTGGACCGGCTGATGAATCCTCTGATTGACCAGTACCTGTACTACTTGTC
TCGGACTCAAACAACAGGAGGCACGGCAAATACGCAGACTCTGGGCTTCAGCCAAGGTGGGCCTAAT
ACAATGGCCAATCAGGCAAAGAACTGGCTGCCAGGACCCTGTTACCGCCAACAACGCGTCTCAACGA
CAACCGGGCAAAACAACAATAGCAACTTTGCCTGGACTGCTGGGACCAAATACAATCTGAATGGAAG
AAATTCATTGGCTAATCCTGGCATCGCTATGGCCTCACACAAAGACGACAAGGAGCGTTTTTTTCCC
AGTAACGGGATCCTGATTTTTGGCAAACAAAATGCTGCCAGAGACAATGCGGATTACAGCGATGTCA
TGCTCACCAGCGAGGAAGAAATCAAAACCACTAACCCTGTGGCTACAGAGGAATACGGTATCGTGGC
AGATAACTTGCAGCAGCAAAACACGGCTCCTCAAATTGGAACTGTCAACAGCCAGGGGGCCTTACCC
GGTATGGTCTGGCAGAACCGGGACGTGTACCTGCAGGGTCCCATCTGGGCCAAGATTCCTCACACGG
ACGGCAACTTCCACCCGTCTCCGCTGATGGGCGGCTTTGGCCTGAAACATCCTCCGCCTCAGATCCT
GATCAAGAACACGCCTGTACCTGCGGATCCTCCGACCACCTTCAACCAGTCAAAGCTGAACTCTTTC
ATCACGCAATACAGCACCGGACAGGTCAGCGTGGAAATTGAATGGGAGCTGCAGAAGGAAAACAGCA
AGCGCTGGAACCCCGAGATCCAGTACACCTCCAACTACTACAAATCTACAAGTGTGGACTTTGCTGT
TAATACAGAAGGCGTGTACTCTGAACCCCACCCCATTGGCACCCGTTACCTCACCCGTCCCCTGTAA
LP2-20 nucleotide sequence
(SEQ ID NO: 8)
ATGGCTGCCGATGGTTATCTTCCAGATTGGCTCGAGGACACTCTCTCTGAAGGAATAAGACAGTGGT
GGAAGCTCAAACCTGGCCCACCACCACCAAAGCCCGCAGAGCGGCATAAGGACGACAGCAGGGGTCT
TGTGCTTCCTGGGTACAAGTACCTCGGACCCTTCAACGGACTCGACAAGGGAGAGCCGGTCAACGAG
GCAGACGCCGCGGCCCTCGAGCACGACAAAGCCTACGACCGGCAGCTCGACAGCGGAGACAACCCGT
ACCTCAAGTACAACCACGCCGACGCGGAGTTTCAGGAGCGCCTTAAAGAAGATACGTCTTTTGGGGG
CAACCTCGGACGAGCAGTCTTCCAGGCGAAAAAGAGGGTTCTTGAACCTCTGGGCCTGGTTGAGGAA
CCTGTTAAGACGGCTCCGGGAAAAAAGAGGCCGGTAGAGCACTCTCCTGTGGAGCCAGACTCCTCCT
CGGGAACCGGAAAGGCGGGCCAGCAGCCTGCAAGAAAAAGATTGAATTTTGGTCAGACTGGAGACGC
AGACTCAGTACCTGACCCCCAGCCTCTCGGACAGCCACCAGCAGCCCCCTCTGGTCTGGGAACTAAT
ACGATGGCTACAGGCAGTGGCGCACCAATGGCAGACAATAACGAGGGCGCCGACGGAGTGGGTAATT
CCTCGGGAAATTGGCATTGCGATTCCACATGGATGGGCGACAGAGTCATCACCACCAGCACCCGAAC
CTGGGCCCTGCCCACCTACAACAACCACCTCTACAAACAAATTTCCAGCCAATCAGGAGCCTCGAAC
GACAATCACTACTTTGGCTACAGCACCCCTTGGGGGTATTTTGACTTCAACAGATTCCACTGCCACT
TTTCACCACGTGACTGGCAAAGACTCATCAACAACAACTGGGGATTCCGACCCAAGAGACTCAACTT
CAAGCTCTTTAACATTCAAGTCAAAGAGGTCACGCAGAATGACGGTACGACGACGATTGCCAATAAC
CTTACCAGCACGGTTCAGGTGTTTACTGACTCGGAGTACCAGCTCCCGTACGTCCTCGGCTCGGCGC
ATCAAGGATGCCTCCCGCCGTTCCCAGCAGACGTCTTCATGGTGCCACAGTATGGATACCTCACCCT
GAACAACGGGAGTCAGGCAGTAGGACGCTCTTCATTTTACTGCCTGGAGTACTTTCCTTCTCAGATG
CTGCGTACCGGAAACAACTTTACCTTCAGCTACACTTTTGAGGACGTTCCTTTCCACAGCAGCTACG
CTCACAGCCAGAGTCTGGACCGTCTCATGAATCCTCTCATCGACCAGTACCTGTATTACTTGAGCAG
AACAAACACTCCAAGTGGAACCACCACGCAGTCAACGCTTCAGTTTTCTCAGGCCGGAGCGAGTGAC
ATTCGGGACCAGTCTAGGAACTGGCTTCCTGGACCCTGTTACCGCCAGCAGCGAGTATCAAAGACAT
CTGCGGATAACAACAACAGTGAATACTCGTGGACTGGAGCTACCAAGTACCACCTCAATGGCAGAGA
CTCTCTGGTGAATCCGGGCCCGGCCATGGCAAGCCACAAGGACGATGAAGAAAAGTTTTTTCCTCAG
AGCGGGGTTCTCATCTTTGGGAAGCAAGGCTCAGAGAAAACAAATGTGGACATTGAAAAGGTCATGA
TTACAGACGAAGAGGAAATCAGGACAACCAATCCCGTGGCTACGGAGCAGTATGGTTCTGTATCTAC
CAACCTCCAGAGAGGCAACAGACAAGCAGCTACCGCAGATGTCAACACACAAGGCGTTCTTCCAGGC
ATGGTCTGGCAGGACAGAGATGTGTACCTTCAGGGGCCCATCTGGGCAAAGATTCCACACACGGACG
GACATTTTCACCCCTCTCCCCTCATGGGTGGATTCGGACTTAAACACCCTCCTCCACAGATTCTCAT
CAAGAACACCCCGGTACCTGCGAATCCTTCGACCACCTTCAGTGCGGCAAAGTTTGCTTCCTTCATC
ACACAGTACTCCACGGGACAGGTCAGCGTGGAGATCGAGTGGGAGCTGCAGAAGGAAAACAGCAAAC
GCTGGAATCCCGAAATTCAGTACACTTCCAACTACAACAAGTCTGTTAATGTGGACTTTACTGTGGA
CACTAATGGCGTGTATTCAGAGCCTCGCCCCATTGGCACCAGATACCTGACTCGTAATCTGTAA
LP3-19 nucleotide sequence
(SEQ ID NO: 9)
ATGGCTGCCGATGGTTATCTTCCAGATTGGCTCGAGGACACTCTCTCTGAAGGAATAAGACAGTGGT
GGAAGCTCAAACCTGGCCCACCACCACCAAAGCCCGCAGAGCGGCATAAGGACGACAGCAGGGGTCT
TGTGCTTCCTGGGTACAAGTACCTCGGACCCTTCAACGGACTCGACAAGGGAGAGCCGGTCAACGAG
GCAGACGCCGCGGCCCTCGAGCACGACAAAGCCTACGACCGTCAGCTCGACAGCGGAGACAACCCGT
ACCTCAAGTATAACCACGCCGACGCCGAGTTTCAGGAGCGTCTGCAAGAAGATACGTCTTTTGGGGG
CAACCTCGGGCGAGCAGTCTTCCAGGCCAAGAAGCGGGTTCTCGAACCTCTCGGTCTGGTTGAGGAA
GGCGCTAAGACGGCTCCTGGAAAGAAACGTCCGGTAGAGCAGTCGCCACAAGAGCCAGACTCCTCCT
CGGGCATTGGCAAGACAGGCCAGCAGCCCGCTAAAAAGAGACTCAATTTTGGTCAGACTGGCGACTC
AGAGTCAGTCCCCGACCCACAACCTCTCGGAGAACCTCCAGCAACCCCCGCTGCTGTGGGACCTACT
ACAATGGCTTCAGGCGGTGGCGCACCAATGGCGGACAATAACGAGGGCGCCGACGGAGTGGGTAATT
CCTCGGGAAATTGGCATTGCGATTCCACATGGCTGGGCGACAGAGTCATCACCACCAGCACCCGAAC
CTGGGCCCTGCCCACCTACAACAACCACCTCTACAAACAAATTTCCAGCCAATCAGGAGCCTCGAAC
GACAATCACTACTTTGGCTACAGCACCCCTTGGGGGTATTTTGACTTCAACAGATTCCACTGCCACT
TTTCACCACGTGACTGGCAGCGACTCATCAACAACAACTGGGGATTCCGGCCTAAGCGACTCAACTT
CAAGCTCTTCAACATTCAGGTCAAAGAGGTTACGGACAACAATGGAGTCAAGACCATCGCCAATAAC
CTTACCAGCACGGTCCAGGTCTTCACGGACTCGGAGTACCAGTTGCCGTACGTCCTCGGCTCTGCGC
ACCAGGGCTGCCTCCCTCCGTTCCCGGCGGACGTGTTCATGATTCCGCAGTACGGCTACCTAACGCT
CAACAATGGCAGCCAGGCAGTGGGACGGTCATCCTTTTACTGCCTGGAATATTTCCCATCGCAGATG
CTGAGAACGGGCAACAACTTTACCTTCAGCTACACCTTCGAGGACGTGCCTTTCCACAGCAGCTACG
CCCACAGCCAGAGCTTGGACCGGCTGATGAATCCTCTGATTGACCAGTACCTGTACTACTTGTCTCG
GACTCAAACAACAGGAGGCACGGCAAATACGCAGACTCTGGGCTTCAGCCAAGGTGGGCCTAATACA
ATGGCCAATCAGGCAAAGAACTGGCTGCCAGGACCCTGTTACCGCCAACAACGCGTCTCAACGACAA
CCGGGCAAAACAACAATAGCAACTTTGCCTGGACTGCTGGGACCAAATACCATCTGAATGGAAGAAA
TTCATTGGCTAATCCTGGCATCGCTATGGCAACACACAAAGACGACGAGGAGCGTTTTTTTCCCAGT
AACGGGATCCTGATTTTTGGCAAACAAAATGCTGCCAGAGACAATGCGGATTACAGCGATGTCATGC
TCACCAGCGAGGAAGAAATCAAAACCACTAACCCTGTGGCTACAGAGGAATACGGTATCGTGGCAGA
TAACTTGCAGCAGCAAAACACGGCTCCTCAAATTGGAACTGTCAACAGCCAGGGGGCCTTACCCGGT
ATGGTTTGGCAGAACCGGGACGTGTACCTGCAGGGTCCCATCTGGGCCAAGATTCCTCACACGGACG
GCAACTTCCACCCGTCTCCGCTGATGGGCGGCTTTGGCCTGAAACATCCTCCGCCTCAGATCCTGAT
CAAGAACACGCCTGTACCTGCGGATCCTCCGACCACCTTCAACCAGTCAAAGCTGAACTCTTTCATC
ACGCAATACAGCACCGGACAGGTCAGCGTGGAAATTGAATGGGAGCTGCAGAAGGAAAACAGCAAGC
GCTGGAACCCCGAGATCCAGTACACCTCCAACTACTACAAATCTACAAGTGTGGACTTTGCTGTTAA
TACAGAAGGCGTGTACTCTGAACCCCGCCCCATTGGCACCCGTTACCTCACCCGTAATCTGTAA
LP4-7 nucleotide sequence
(SEQ ID NO: 10)
ATGGCTGCCGATGGTTATCTTCCAGATTGGCTCGAGGACAACCTCTCTGAGGGCATTCGCGAGTGGT
GGGACTTGAAACCTGGAGCCCCGAAACCCAAAGCCAACCAGCAAAAGCAGGACGACGGCCGGGGTCT
GGTGCTTCCTGGCTACAAGTACCTCGGACCCTTCAACGGACTCGACAAGGGGGAGCCCGTCAACGCG
GCGGATGCAGCGGCCCTCGAGCACGACAAAGCCTACGACCGGCAGCTCGACAGCGGAGACAACCCGT
ACCTCAAGTACAACCACGCCGACGCCGAGTTCCAGGAGCGGCTCAAAGAAGATACGTCTTTTGGGGG
CAACCTCGGGCGAGCAGTCTTCCAGGCCAAAAAGAGGCTTCTTGAACCTCTTGGTCTGGTTGAGGAA
GCGGCTAAGACGGCTCCTGGAAAGAAACGTCCGGTAGAGCAGTCGCCACAAGAGCCAGACTCCTCCT
CGGGCATCGGCAAGACAGGCCAGCAGCCCGCTAAAAAGAGACTCAATTTTGGTCAGACTGGCGACTC
AGAGTCAGTCCCCGATCCACAACCTCTCGGAGAACCTCCAGCAACCCCCGCTGCTGTGGGACCTACT
ACAATGGCTTCAGGCGGTGGCGCACCAATGGCAGACAATAACGAAGGCGCCGACGGAGTGGGTAGTT
CCTCGGGAAATTGGCATTGCGATTCCCAATGGCTGGGGGACAGAGTCATCACCACCAGCACCCGAAC
CTGGGCCCTGCCCACCTACAACAACCACCTCTACAAGCAAATCTCCAACAGCACATCTGGAGGAGCC
ACCAACGACAACACCTACTTCGGCTACAGCACCCCCTGGGGGTATTTTGACTTTAACAGATTCCACT
GCCACTTCTCACCACGTGACTGGCAGCGACTCATCAACAACAACTGGGGATTCCGGCCTAAGCGACT
CAACTTCAAGCTCTTCAACATTCAGGTCAAAGAGGTTACGGACAACAATGGAGTCAAGACCATCGCC
AATAACCTTACCAGCACGGTCCAGGTCTTCACGGACTCGGAGTACCAGTTGCCGTACGTCCTCGGCT
CTGCGCACCAGGGCTGCCTCCCTCCGTTCCCGGCGGACGTGTTCATGATTCCTCAGTACGGGTACCT
GACTCTGAACAACGGCAGTCAGGCCGTGGGCCGTTCCTCCTTCTACTGCCTGGAGTACTTTCCTTCT
CAAATGCTGAGAACGGGCAACAACTTCCAGTTTACTTACACCTTCGAGGACGTGCCTTTCCACAGCA
GCTACGCTCACAGCCAGAGTCTGGACCGTCTCATGAATCCTCTGATTGACCAGTACCTGTACTACTT
GTCTCGGACTCAAACAACAGGAGGCACGGCAAATACGCAGACTCTGGGCTTCAGCCAAGGTGGGCCT
AATACAATGGCCAATCAGGCAAAGAACTGGCTGCCAGGACCCTGTTACCGCCAACAACGCGTCTCAA
CGACAACCGGGCAAAACAACAATAGCAACTTTGCCTGGACTGCTGGGACCAAATACCATCTGAATGG
AAGAAATTCATTGGCTAATCCTGGCATCGCTATGGCCTCACACAAAGACGACAAGGAGCGTTTTTTT
CCCAGTAACGGGATCCTGATTTTTGGCAAACAAAATGCTGCCAGAGACAATGCGGATTACAGCGATG
TCATGCTCACCAGCGAGGAAGAAATCAAAACCACTAACCCTGTGGCTACAGAGGAATACGGTATCGT
GGCAGATAACTTGCAGCAGCAAAACACGGCTCCTCAAATTGGAACTGTCAACAGCCAGGGGGCCTTA
CCCGGTATGGTCTGGCAGAACCGGGACGTGTACCTGCAGGGTCCCATCTGGGCCAAGATTCCTCACA
CGGACGGCAACTTCCACCCGTCTCCGCTGATGGGCGGCTTTGGCCTGAAACATCCTCCGCCTCAGAT
CCTGATCAAGAACACGCCTGTACCTGCGGATCCTCCGACCACCTTCAACCAGTCAAAGCTGAACTCT
TTCATCACGCAATACAGCACCGGACAGGTCAGCGTGGAAATTGAATGGGAGCTGCAGAAGGAAAACA
GCAAGCGCTGGAACCCCGAGATCCAGTACACCTCCAACTACTACAAATCTACAAGTGTGGACTTTGC
TGTTAATACAGAAGGCGTGTACTCTGAACCCCACCCCATTGGCACCCGTTACCTTACCCGTCCCCTG
TAA
LP4-15 nucleotide sequence
(SEQ ID NO: 11)
ATGGCTGCCGATGGTTATCTTCCAGATTGGCTCGAGGACAACCTTAGTGAAGGAATTCGCGAGTGGT
GGGCTTTGAAACCTGGAGCCCCGAAGCCCAAAGCCAACCAGCAAAAGCAGGACGACGGCCGGGGTCT
GGTGCTTCCTGGCTACAAGTACCTCGGACCCTTCAACGGACTCGACAAAGGAGAGCCCGTCAACGCG
GCGGATGCAGCGGCCCTCGAGCACGACAAAGCCTACGACCGGCAGCTCGACAGCGGAGACAACCCGT
ACCTCAAGTACAACCACGCCGACGCGGAGTTTCAGGAGCGCCTTAAAGAAGATACGTCTTTTGGGGG
CAACCTCGGACGAGCAGTCTTCCAGGCGAAAAAGAGGGTTCTTGAACCTCTGGGCCTGGTTGAGGAA
CCTGTTAAGACGGCTCCGGGAAAAAAGAGGCCGGTAGAGCACTCTCCTGTGGAGCCAGACTCCTCCT
CGGGAACCGGAAAGGCGGGCCAGCAGCCTGCAAGAAAAAGATTGAATTTTGGTCAGACTGGAGACGC
AGACTCAGTACCTGACCCCCAGCCTCTCGGACAGCCACCAGCAGCCCCCTCTGGTCTGGGAACTAAT
ACGATGGCTACAGGCAGTGGCGCACCAATGGCAGACAATAACGAGGGCGCCGACGGAGTGGGTAATT
CCTCGGGAAATTGGCATTGCGATTCCACATGGATGGGCGACAGAGTCATCACCACCAGCACCCGAAC
CTGGGCCCTGCCCACCTACAACAACCACCTCTACAAACAAATTTCCAGCCAATCAGGAGCCTCGAAC
GACAATCACTACTTTGGCTACAGCACCCCTTGGGGGTATTTTGACTTCAACAGATTCCACTGCCACT
TTTCACCACGTGACTGGCAAAGACTCATCAACAACAACTGGGGATTCCGACCCAAGAGACTCAACTT
CAAGCTCTTTAACATTCAAGTCAAAGAGGTCACGCAGAATGACGGTACGACGACGATTGCCAATAAC
CTTACCAGCACGGTTCAGGTGTTTACTGACTCGGAGTACCAGCTCCCGTACGTCCTCGGCTCGGCGC
ATCAAGGATGCCTCCCGCCGTTCCCAGCAGACGTCTTCATGGTGCCACAGTATGGATACCTCACCCT
GAACAACGGGAGTCAGGCAGTAGGACGCTCTTCATTTTACTGCCTGGAGTACTTTCCTTCTCAGATG
CTGCGTACCGGAAACAACTTTACCTTCAGCTACACTTTTGAGGACGTTCCTTTCCACAGCAGCTACG
CTCACAGCCAGAGTCTGGACCGTCTCATGAATCCTCTCATCGACCAGTACCTGTATTACTTGAGCAG
AACAAACACTCCAAGTGGAACCACCACGCAGTCAACGCTTCAGTTTTCTCAGGCCGGAGCGAGTGAC
ATTCGGGACCAGTCTAGGAACTGGCTTCCTGGACCCTGTTACCGCCAGCAGCGAGTATCAAAGACAT
CTGCGGATAACAACAACAGTGAATACTCGTGGACTGGAGCTACCAAGTACCACCTCAATGGCAGAGA
CTCTCTGGTGAATCCGGGCCCGGCCATTGCAAGCCACAAGGACGATGAAGAAAAGTTTTTTCCTCAG
AGCGGGGTTCTCATCTTTGGGAAGCAAGGCTCAGAGAAAACAAATGTGGACATTGAAAAGGTCATGA
TTACAGACGAAGAGGAAATCAGGACAACCAATCCCGTGGCTACGGAGCAGTATGGTTCTGTATCTAC
CAACCTCCAGAGAGGCAACAGACAAGCAGCTACCGCAGATGTCAACACACAAGGCGTTCTTCCAGGC
ATGGTCTGGCAGGACAGAGATGTGTACCTTCAGGGGCCCATCTGGGCAAAGATTCCACACACGGACG
GACATTTTCACCCCTCTCCCCTCATGGGTGGATTCGGACTTAAACACCCTCCTCCACAGATTCTCAT
CAAGAACACCCCGGTACCTGCGAATCCTTCGACCACCTTCAGTGCGGCAAAGTTTGCTTCCTTCATC
ACACAGTACTCCACGGGACAGGTCAGCGTGGAGATCGAGTGGGAGCTGCAGAAGGAAAACAGCAAAC
GCTGGAATCCCGAAATTCAGTACACTTCCAACTACAACAAGTCTGTTAATGTGGACTTTACTGTGGA
CACTAATGGCGTGTATTCAGAGCCTCGCCCCATTGGCACCAGATACCTGACTCGTAATCTGTAA
LP4-19 nucleotide sequence
(SEQ ID NO: 12)
ATGGCTGCCGATGGTTATCTTCCAGATTGGCTCGAGGACAACCTTAGTGAAGGAATTCGCGAGTGGT
GGGCTTTGAAACCTGGAGCCCCGAAGCCCAAAGCCAACCAGCAAAAGCAGGACGACGGCCGGGGTCT
GGTGCTTCCTGGCTACAAGTACCTCGGACCCTTCAACGGACTCGACAAGGGGGAGCCCGTCAACGCG
GCGGACGCAGCGGCCCTCGAGCACGACAAGGCCTACGACCAGCAGCTCAAAGCGGGTGACAATCCGT
ACCTGCGGTATAACCACGCCGACGCCGAGTTTCAGGAGCGTCTGCAAGAAGATACGTCTTTTGGGGG
CAACCTCGGGCGAGCAGTCTTCCAGGCCAAGAAGCGGGTTCTCGAACCTCTCGGTCTGGTTGAGGAA
GGCGCTAAGACGGCTCCTGGAAAGAAACGTCCGGTAGAGCAGTCGCCACAAGAGCCAGACTCCTCCT
CGGGCATCGGCAAGACAGGCCAGCAGCCCGCTAAAAAGAGACTCAATTTTGGTCAGACTGGCGACTC
AGAGTCAGTCCCCGACCCACAACCTCTCGGAGAACCTCCAGCAACCCCCGCTGCTGTGGGACCTACT
ACAATGGCTTCAGGCGGTGGCGCACCAATGGCGGACAATAACGAGGGCGCCGACGGAGTGGGTAATT
CCTCGGGAAATTGGCATTGCGATTCCACATGGCTGGGCGACAGAGTCATCACCACCAGCACCCGAAC
CTGGGCCCTGCCCACCTACAACAACCACCTCTACAAACAAATTTCCAGCGCTTCAACGGGAGCCTCG
AACGACAATCACTACTTTGGCTACAGCACCCCTTGGGGGTATTTTGACTTCAACAGATTCCACTGCC
ACTTTTCACCACGTGACTGGCAGCGACTCATCAACAACAACTGGGGATTCCGGCCTAAGCGACTCAA
CTTCAAGCTCTTCAACATTCAGGTCAAAGAGGTTACGGACAACAATGGAGTCAAGACCATCGCCAAT
AACCTTACCAGCACGGTCCAGGTCTTCACGGACTCGGAGTACCAGTTGCCGTACGTCCTCGGCTCTG
CGCACCAGGGCTGCCTCCCTCCGTTCCCGGCGGACGTGTTCATGATTCCGCAGTACGGCTACCTGAC
ACTCAACAATGGCAGCCAGGCAGTGGGACGGTCATCCTTTTACTGCCTGGAATATTTCCCATCGCAG
ATGCTGAGAACCGGCAACAACTTCCAGTTTACTTACACCTTCAAGGACGTGCCTTTCCACAGCAGCT
ACGCGCACAGCCAGAGCTTGGACCGGCTGATGAATCCTCTGATTGACCAGTACCTGTACTACTTGTC
TCGGACTCAAACAACAGGAGGCACGGCAAATACGCAGACTCTGGGCTTCAGCCAAGGTGGGCCTAAT
ACAATGGCCAATCAGGCAAAGAACTGGCTGCCAGGACCCTGTTACCGCCAACAACGCGTCTCAACGA
CAACCGGGCAAAACAACAATAGCAACTTTGCCTGGACTGCTGGGACCAAATACCATCTGAATGGAAG
AAATTCATTGGCTAATCCTGGCATCGCTATGGCAACACACAAAGACGACGAGGAGCGTTTTTTTCCC
AGTAACGGGATCCTGATTTTTGGCAAACAAAATGCTGCCAGAGACAATGCGGATTACAGCGATGTCA
TGCTCACCAGCGAGGAAGAAATCAAAACCACTAACCCTGTGGCTACAGAGGAATACGGTATCGTGGC
AGATAACTTGCAGCAGCAAAACACGGCTCCTCAAATTGGAACTGTCAACAGCCAGGGGGCCTTACCC
GGTATGGTTTGGCAGAACCGGGACGTGTACCTGCAGGGTCCCATCTGGGCCAAGATTCCTCACACGG
ACGGCAACTTCCACCCGTCTCCGCTGATGGGCGGCTTTGGCCTGAAACATCCTCCGCCTCAGATCCT
GATCAAGAACACGCCTGTACCTGCGGATCCTCCGACCACCTTCAACCAGTCAAAGCTGAACTCTTTC
ATCACGCAATACAGCACCGGACAGGTCAGCGTGGAAATTGAATGGGAGCTGCAGAAGGAAAACAGCA
AGCGCTGGAACCCCGAGATCCAGTACACTTCCAACTACTACAAATCTACAAGTGTGGACTTTGCTGT
TAATACAGAAGGCGTGTACTCTGAACCCCGCCCCATTGGCACCCGTTACCTCACCCGTCCCCTGTAA
LP4-21 nucleotide sequence
(SEQ ID NO: 13)
ATGGCTGCCGATGGTTATCTTCCTGATTGGCTCGAGGACACTCTCTCTGAAGGAATAAGACAGTGGT
GGAAGCTCAAACCTGGCCCACCACCACCAAAGCCCGCAGAGCGGCATAAGGACGACAGCAGGGGTCT
TGTGCTTCCTGGGTACAAGTACCTCGGACCCTTCAACGGACTCGACAAGGGGGAGCCCGTCAACGCG
GCGGATGCAGCGGCCCTCGAGCACGACAAGGCCTACGACCAGCAGCTCAAAGCGGGTGACAATCCGT
ACCTGCGGTATAACCACGCCGACGCCGAGTTTCAGGAGCGTCTGCAAGAAGATACGTCTTTTGGGGG
CAACCTCGGGCGAGCAGTCTTCCAGGCCAAGAAGAGGGTTCTCGAACCTTTTGGTCTGGTTGAGGAA
GGTGCTAAGACGGCTCCTGGAAAGAAACGTCCGGTAGAGCAGTCGCCACAAGAGCCAGACTCCTCCT
CGGGCATCGGCAAGACAGGCCAGCAGCCCGCTAAAAAGAGACTCAATTTTGGTCAGACTGGCGACTC
AGAGTCAGTCCCCGACCCACAACCTCTCGGAGAACCTCCAGCAACCCCCGCTGCTGTGGGACCTACT
ACAATGGCTTCAGGCGGTGGCGCACCAATGGCGGACAATAACGAGGGCGCCGACGGAGTGGGTAATT
CCTCGGGAAATTGGCATTGCGATTCCACATGGCTGGGCGACAGAGTCATCACCACCAGCACCCGAAC
CTGGGCCCTGCCCACTTACAACAACCATCTCTACAAGCAAATCTCCAGTGCTTCAACGGGGGCCAGC
AACGACAACCACTACTTCGGCTACAGCACCCCCTGGGGGTATTTTGACTTCAACAGATTCCACTGCC
ACTTCTCACCACGTGACTGGCAGCGACTCATCAACAACAACTGGGGATTCCGGCCTAAGCGACTCAA
CTTCAAACTCTTCAACATTCAGGTCAAAGAGGTTACGGACAACAATGGAGTCAAGACCATCGCCAAT
AACCTTACCAGCACGGTCCAGGTCTTCACGGACTCGGAGTACCAGTTGCCGTACGTCCTCGGCTCTG
CGCACCAGGGCTGCCTCCCTCCGTTCCCGGCGGACGTGTTCATGATTCCGCAGTACGGCTACCTAAC
GCTCAACAATGGCAGCCAGGCAGTGGGACGGTCATCCTTCTACTGCCTGGAATACTTTCCTTCGCAG
ATGCTGAGAACCGGCAACAACTTCCAGTTTACTTACACCTTCGAGGACGTGCCTTTCCACAGCAGCT
ACGCCCACAGCCAGAGCTTGGACCGGCTGATGAATCCTCTGATTGACCAGTACCTGTACTACTTGTC
TCGGACTCAAACAACAGGAGGCACGGCAAATACGCAGACTCTGGGCTTCAGCCAAGGTGGGCCTAAT
ACAATGGCCAATCAGGCAAAGAACTGGCTGCCAGGACCCTGTTACCGCCAACAACGCGTCTCAACGA
CAACCGGGCAAAATAACAATAGCAACTTTGCCTGGACTGCTGGGACCAAATACCATCTGAATGGAAG
AAATTCATTGGCTAATCCTGGCATCGCTATGGCCTCACACAAAGACGACAAGGAGCGTTTTTTTCCC
AGTAACGGGATCCTGATTTTTGGCAAACAAAATGCTGCCAGAGACAATGCGGATTACAGCGATGTCA
TGCTCACCAGCGAGGAAGAAATCAAAACCACTAACCCTGTGGCTACAGAGGAATACGGTATCGTGGC
AGATAACTTGCAGCAGCAAAACACGGCTCCTCAAATTGGAACTGTCAACAGCCAGGGGGCCTTACCC
GGTATGGTCTGGCAGAACCGGGACGTGTACCTGCAGGGTCCCATCTGGGCCAAGATTCCTCACACGG
ACGGCAACTTCCACCCGTCTCCGCTGATGGGCGGCTTTGGCCTGAAACATCCTCCGCCTCAGATCCT
GATCAAGAACACGCCTGTACCTGCGGATCCTCCGACCACCTTCAACCAGTCAAAGCTGAACTCTTTC
ATCACGCAATACAGCACCGGACAGGTCAGCGTGGAAATTGAATGGGAGCTGCAGAAGGAAAACAGCA
AGCGCTGGAACCCCGAGATCCAGTACACCTCCAACTACTACAAATCTACAAGTGTGGACTTTGCTGT
TAATACAGAAGGCGTGTACTCTGAACCCCACCCCATTGGCACCCGTTACCTCACCCGTAATCTGTAA
LP4-24 nucleotide sequence
(SEQ ID NO: 14)
ATGGCTGCCGATGGTTATCTTCCAGATTGGCTCGAGGACACTCTCTCTGAAGGAATAAGACAGTGGT
GGAAGCTCAAACCTGGCCCACCACCACCAAAGCCCGCAGAGCGGCATAAGGACGACAGCAGGGGTCT
TGTGCTTCCTGGCTACAAGTACCTCGGACCCTTCAACGGACTCGACAAGGGGGAGCCCGTCAACGCG
GCGGATGCAGCGGCCCTCGAGCACGACAAGGCCTACGACCGGCAGCTCAAAGCGGGTGACAATCCGT
ACCTGCGGTATAACCACGCCGACGCGGAGTTTCAGGAGCGCCTTAAAGAAGATACGTCTTTTGGGGG
CAACCTCGGGCGAGCAGTCTTCCAGGCCAAGAAGAGGGTTCTCGAACCTTTTGGTCTGGTTGAGGAA
GGCGCTAAGACGGCTCCTGGAAAGAAGAGGCCTGTAGAGCAGTCGCCACAAGAGCCAGACTCCTCCT
CGGGCATTGGCAAGACAGGCCAGCAGCCCGCTAAAAAGAGACTCAATTTTGGTCAGACTGGCGACAC
AGAGTCAGTCCCAGACCCTCAACCAATCGGAGAACCTCCCGCAGCCCCCTCAGGTGTGGGATCTCTT
ACAATGGCTTCAGGTGGTGGCGCACCAGTGGCAGACAATAACGAAGGTGCCGATGGAGTGGGTAGTT
CCTCGGGAAATTGGCATTGCGATTCCCAATGGCTGGGCGACAGAGTCATCACCACCAGCACCCGAAC
CTGGGCCCTGCCCACTTACAACAACCATCTCTACAAGCAAATCTCCAGTGCTTCAACGGGGGCCAGC
AACGACAACCACTACTTCGGCTACAGCACCCCCTGGGGGTATTTTGACTTCAACAGATTCCACTGCC
ACTTCTCACCACGTGACTGGCAGCGACTCATCAACAACAACTGGGGATTCCGGCCTAAGCGACTCAA
CTTCAAGCTCTTCAACATTCAGGTCAAAGAGGTTACGGACAACAATGGAGTCAAGACCATCGCCAAT
AACCTTACCAGCACGGTCCAGGTCTTCACGGACTCGGAGTACCAGTTGCCGTACGTCCTCGGCTCTG
CGCACCAGGGCTGCCTCCCTCCGTTCCCGGCGGACGTGTTCATGATTCCGCAGTACGGCTACCTAAC
GCTCAACAATGGCAGCCAGGCAGTGGGACGGTCATCCTTTTACTGCCTGGAATATTTCCCATCGCAG
ATGCTGAGAACCGGCAACAACTTCCAGTTTACTTACACCTTCGAGGACGTGCCTTTCCACAGCAGCT
ACGCCCACAGCCAGAGCTTGGACCGGCTGATGAATCCTCTGATTGACCAGTACCTGTACTACTTGTC
TCGGACTCAAACAACAGGAGGCACGGCAAATACGCAGACTCTGGGCTTCAGCCAAGGTGGGCCTAAT
ACAATGGCCAATCAGGCAAAGAACTGGCTGCCAGGACCCTGTTACCGCCAACAACGCGTCTCAACGA
CAACCGGGCAAAACAACAATAGCAACTTTGCCTGGACTGCTGGGACCAAATACCATCTGAATGGAAG
AAATTCATTGGCTAATCCTGGCATCGCTATGGCCTCACACAAAGACGACAAGGAGCGTTTTTTTCCC
AGTAACGGGATCCTGATTTTTGGCAAACAAAATGCTGCCAGAGACAATGCGGATTACAGCGATGTCA
TGCTCACCAGCGAGGAAGAAATCAAAACCACTAACCCTGTGGCTACAGAGGAATACGGTATCGTGGC
AGATAACTTGCAGCAGCAAAACACGGCTCCTCAAATTGGAACTGTCAACAGCCAGGGGGCCTTACCC
GGTATGGTCTGGCAGAACCGGGACGTGTACCTGCAGGGTCCCATCTGGGCCAAGATTCCTCACACGG
ACGGCAACTTCCACCCGTCTCCGCTGATGGGCGGCTTTGGCCTGAAACATCCTCCGCCTCAGATCCT
GATCAAGAACACGCCTGTACCTGCGGATCCTCCGACCACCTTCAACCAGTCAAAGCTGAACTCTTTC
ATCACGCAATACAGCACCGGACAGGTCAGCGTGGAAATTGAATGGGAGCTGCAGAAGGAAAACAGCA
AGCGCTGGAACCCCGAGATCCAGTACACCTCCAACTACTACAAATCTACAAGTGTGGACTTTGCTGT
TAATACAGAAGGCGTGTACTCTGAACCCCGCCCCATTGGCACCCGTTACCTCACCCGTAATCTGTAA
LP1-1 amino acid sequence
(SEQ ID NO: 15)
MAADGYLPDWLEDNLSEGIREWWDLKPGAPKPKANQQKQDDGRGLVLPGYKYLGPFNGLDKGEPVNA
ADAAALEHDKAYDQQLKAGDNPYLRYNHADAEFQERLQEDTSFGGNLGRAVFQAKKRVLEPFGLVEE
GAKTAPGKKRPVEQSPQEPDSSSGIGKTGQQPAKKRLNFGQTGDTESVPDPQPIGEPPAAPSGVGSL
TMASGGGAPVADNNEGADGVGSSSGNWHCDSQWLGDRVITTSTRTWALPTYNNHLYKQISSASTGAS
NDNHYFGYSTPWGYFDFNRFHCHFSPRDWQRLINNNWGFRPKRLNFKLFNIQVKEVTDNNGVKTIAN
NLTSTVQVFTDSEYQLPYVLGSAHQGCLPPFPADVFMIPQYGYLTLNNGSQAVGRSSFYCLEYFPSQ
MLRTGNNFQFTYTFEDVPFHSSYAHSQSLDRLMNPLIDQYLYYLSRTQTTGGTANTQTLGFSQGGPN
TMANQAKNWLPGPCYRQQRVSTTTGQNNNSNFAWTAGTKYHLNGRNSLANPGIAMASHKDDKERFFP
SNGILIFGKQNAARDNADYSDVMLTSEEEIKTTNPVATEEYGIVADNLQQQNTAPQIGTVNSQGALP
GMVWQNRDVYLQGPIWAKIPHTDGNFHPSPLMGGFGLKHPPPQILIKNTPVPADPPTTFNQSKLNSF
ITQYSTGQVSVEIEWELQKENSKRWNPEIQYTSNYYKSTSVDFAVNTEGVYSEPHPIGTRYLTRPL*
LP1-11 amino acid sequence
(SEQ ID NO: 16)
MAADGYLPDWLEDTLSEGIRQWWKLKPGPPPPKPAERHKDDSRGLVLPGYKYLGPFNGLDKGEPVNA
ADAAALEHDKAYDQQLQAGDNPYLRYNHADAEFQERLQEDTSFGGNLGRAVFQAKKRLLEPLGLVEE
AAKTAPGKKRPVEQSPQEPDSSAGIGKSGAQPAKKRLNFGQTGDTESVPDPQPIGEPPAAPSGVGSL
TMASGGGAPVADNNEGADGVGSSSGNWHCDSTWLGDRVITTSTRTWALPTYNNHLYKQISSASTGAS
NDNHYFGYSTPWGYFDFNRFHCHFSPRDWQRLINNNWGFRPKRLNFKLFNIQVKEVTDNNGVKTIAN
NLTSTVQVFTDSEYQLPYVLGSAHQGCLPPFPADVFMIPQYGYLTLNNGSQAVGRSSFYCLEYFPSQ
MLRTGNNFTFSYTFEDVPFHSSYAHSQSLDRLMNPLIDQYLYYLSRTQTTGGTANTQTLGFSQGGPN
TMANQAKNWLPGPCYRQQRVSTTTGQNNNSNFAWTAGTKYHLNGRNSLANPGIAMASHKDDKERFFP
SNGILIFGKQNAARDNKIPHTDGNFHPSPLMGGFGLKHPPPQILIKNTPVPADPPTTFNQSKLNSFI
TQYSTGQVSVEIEWELQKENSKRWNPEIQYTSNYYKSTSVDFAVNTEGVYSEPRPIGTRYLTRNL*
LP1-13 amino acid sequence
(SEQ ID NO: 17)
MAADGYLPDWLEDTLSEGIRQWWKLKPGPPPPKPAERHKDDSRGLVLPGYKYLGPFNGLDKGEPVNE
ADAAALEHDKAYDRQLDSGDNPYLKYNHADAEFQERLKEDTSFGGNLGRAVFQAKKRVLEPLGLVEE
PVKTAPGKKRPVEQSPQEPDSSSGIGKTGQQPAKKRLNFGQTGDSESVPDPQPLGEPPAAPSGVGPN
TMAAGGGAPMADNNEGADGVGSSSGNWHCDSQWLGDRVITTSTRTWTLPTYNNHLYKQISNSTSGGA
TNDNTYFGYSTPWGYFDFNRFHCHFSPRDWQRLINNNWGFRPKRLNFKLFNIQVKEVTDNNGVKTIA
NNLTSTVQVFTDSEYQLPYVLGSAHQGCLPPFPADVFMIPQYGYLTLNNGSQAVGRSSFYCLEYFPS
QMLRTGNNFQFTYTFEDVPFHSSYAHSQSLDRLMNPLIDQYLYYLSRTQTTGGTANTQTLGFSQGGP
NTMANQAKNWLPGPCYRQQRVSTTTGQNNNSNFAWTAGTKYHLNGRNSLANPGIAMATHKDDEERFF
PSNGILIFGKQNAARDNADYSDVMLTSEEEIKTTNPVATEEYGIVADNLQQQNTAPQIGTVNSQGAL
PGMVWQNRDVYLQGPIWAKIPHTDGNFHPSPLMGGFGLKHPPPQILIKNTPVPADPPTTFNQSKLNS
FITQYSTGQVSVEIEWELQKENSKRWNPEIQYTSNYYKSTSVDFAVNTEGVYSEPHPIGTRYLTRP
L*
LP1-18 amino acid sequence
(SEQ ID NO: 18)
MAADGYLPDWLEDNLSEGIREWWALKPGAPKPKANQQKQDDGRGLVLPGYKYLGPFNGLDKGEPVNA
ADAAALEHDKAYDQQLKAGDNPYLRYNHADAEFQERLQEDTSFGGNLGRAVFQAKKRVLEPLGLVEE
GAKTAPGKKRPVEQSPQEPDSSSGIGKTGQQPAKKRLNFGQTGDSESVPDPQPLGEPPATPAAVGPT
TMASGGGAPMADNNEGADGVGSSSGNWHCDSTWLGDRVITTSTRTWALPTYNNHLYKQISSASTGAS
NDNHYFGYSTPWGYFDFNRFHCHFSPRDWQRLINNNWGFRPKRLNFKLFNIQVKEVTDNNGVKTIAN
NLTSTVQVFTDSEYQLPYVLGSAHQGCLPPFPADVFMIPQYGYLTLNDGSQAVGRSSFYCLEYFPSQ
MLRTGNNFQFTYTFEDVPFHSSYAHSQSLDRLMNPLIDQYLYYLSRTQTTGGTANTQTLGFSQGGPN
TMANQAKNWLPGPCYRQQRVSTTTGQNNNSNFAWTAGTKYHLNGRNSLANPGIAMATHKDDKERFFP
SNGILIFGKQNAARDNADYSDVMLTSEEEIKTTNPVATEEYGIVADNLQQQNTAPQIGTVNSQGALP
GMVWQNRDVYLQGPIWAKIPHTDGNFHPSPLMGGFGLKHPPPQILIKNTPVPADPPTTFNQSKLNSF
ITQYSTGQVSVEIEWELQKENSKRWNPEIQYTSNYYKSTSVDFAVNTEGVYSEPRPIGTRYLTRNL*
LP1-23 amino acid sequence
(SEQ ID NO: 19)
MAADGYLPDWLEDNLSEGIREWWALKPGAPKPKANQQKQDDGRGLVLPGYKYLGPFNGLDKGEPVNA
ADAAALEHDKAYDQQLQAGDNPYLRYNHADAEFQERLQEDTSFGGNLGRAVFQAKKRLLEPLGLVEE
AAKTAPGKKRPVEQSPQEPDSSAGIGKSGAQPAKKRLNFGQTGGTESVPDPQPIGEPPAAPSGVGSL
TMASGGGAPVADNNEGADGVGSSSGNWHCDSQWLGDRVITTSTRTWALPTYNNHLYKQISSASTGAS
NDNHYFGYSTPWGYFDFNRFHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLNNGSQAVGRSSFYCLEYFPSQ
MLRTGNNFTFSYTFEDVPFHSSYAHSQSLDRLMNPLIDQYLYYLSRTQTTGGTANTQTLGFSQGGPN
TMANQAKNWLPGPCYRQQRVSTTTGQNNNSNFAWTAGTKYHLNGRNSLANPGIAMASHKDDKERFFP
SNGILIFGKQNAARDNADYSDVMLTSEEEIKTTNPVATEEYGIVADNLQQQNTAPQIGTVNSQGALP
GMVWQNRDVYLQGPIWAKIPHTDGNFHPSPLMGGFGLKHPPPQILIKNTPVPADPPTTFNQSKLNSF
ITQYSTGQVSVEIEWELQKENSKRWNPEIQYTSNYYKSTSVDFAVNTEGVYSEPHPIGTRYLTRPL*
LP2-10 amino acid sequence
(SEQ ID NO: 20)
MAADGYLPDWLEDTLSEGIRQWWKLKPGAPKPKANQQKQDDGRGLVLPGYKYLGPFNGLDKGEPVNA
ADAAALEHDKAYDQQLKAGDNPYLRYNHADAEFQERLQEDTSFGGNLGRAVFQAKKRLLEPLGLVEE
AAKTAPGKKRPVEQSPQEPDSSAGIGKSGAQPAKKRLNFGQTGGTESVPDPQPLGEPPAAPSGVGPN
TMAAGGGAPMADNNEGADGVGSSSGNWHCDSTWLGDRVITTSTRTWALPTYNNHLYKQISSASTGAS
NDNHYFGYSTPWGYFDFNRFHCHFSPRDWQRLINNNWGFRPKRLSFKLFNIQVKEVTQNEGTKTIAN
NLTSTIQVFTDSEYQLPYVLGSAHQGCLPPFPADVFMIPQYGYLTLNNGSQAVGRSSFYCLEYFPSQ
MLRTGNNFQFTYTFEDVPFHSSYAHSQSLDRLMNPLIDQYLYYLSRTQTTGGTANTQTLGFSQGGPN
TMANQAKNWLPGPCYRQQRVSTTTGQNNNSNFAWTAGTKYHLNGRNSLANPGIAMATHKDDKERFFP
SNGILIFGKQNAARDNADYSDVMLTSEEEIKTTNPVATEEYGIVADNLQQQNTAPQIGTVNSQGALP
GMVWQNRDVYLQGPIWAKIPHTDGNFHPSPLMGGFGLKHPPPQILIKNTPVPADPPTTFNQSKLNSF
ITQYSTGQVSVEIEWELQKENSKRWNPEIQYTSNYYKSTSVDFAVNTEGVYSEPHPIGTRYLTRPL*
LP2-19 amino acid sequence
(SEQ ID NO: 21)
MAADGYLPDWLEDTLSEGIRQWWKLKPGPPPPKPAERHKDDSRGLVLPGYKYLGPFNGLDKGEPVNE
ADAAALEHDKAYDRQLDSGDNPYLKYNHADAEFQERLKEDTSFGGNLGRAVFQAKKRVLEPLGLVEE
GAKTAPGKKRPVEQSPQEPDSSAGIGKSGAQPAKKRLNFGQTGDSESVPDPQPLGEPPAAPSGVGPN
TMASGGGAPVADNNEGADGVGSSSGNWHCDSQWLGDRVITTSTRTWALPTYNNHLYKQISSASTGAS
NDNHYFGYSTPWGYFDFNRFHCHFSPRDWQRLINNNWGFRPKRLNFKLFNIQVKEVTDNNGVKTIAN
NLTSTVQVFTDSEYQLPYVLGSAHQGCLPPFPADVFMIPQYGYLTLNNGSQAVGRSSFYCLEYFPSQ
MLRTGNNFQFTYTFEDVPFHSSYAHSQSLDRLMNPLIDQYLYYLSRTQTTGGTANTQTLGFSQGGPN
TMANQAKNWLPGPCYRQQRVSTTTGQNNNSNFAWTAGTKYNLNGRNSLANPGIAMASHKDDKERFFP
SNGILIFGKQNAARDNADYSDVMLTSEEEIKTTNPVATEEYGIVADNLQQQNTAPQIGTVNSQGALP
GMVWQNRDVYLQGPIWAKIPHTDGNFHPSPLMGGFGLKHPPPQILIKNTPVPADPPTTFNQSKLNSF
ITQYSTGQVSVEIEWELQKENSKRWNPEIQYTSNYYKSTSVDFAVNTEGVYSEPHPIGTRYLTRPL*
LP2-20 amino acid sequence
(SEQ ID NO: 22)
MAADGYLPDWLEDTLSEGIRQWWKLKPGPPPPKPAERHKDDSRGLVLPGYKYLGPFNGLDKGEPVNE
ADAAALEHDKAYDRQLDSGDNPYLKYNHADAEFQERLKEDTSFGGNLGRAVFQAKKRVLEPLGLVEE
PVKTAPGKKRPVEHSPVEPDSSSGTGKAGQQPARKRLNFGQTGDADSVPDPQPLGQPPAAPSGLGTN
TMATGSGAPMADNNEGADGVGNSSGNWHCDSTWMGDRVITTSTRTWALPTYNNHLYKQISSQSGASN
DNHYFGYSTPWGYFDFNRFHCHFSPRDWQRLINNNWGFRPKRLNFKLFNIQVKEVTQNDGTTTIANN
LTSTVQVFTDSEYQLPYVLGSAHQGCLPPFPADVFMVPQYGYLTLNNGSQAVGRSSFYCLEYFPSQM
LRTGNNFTFSYTFEDVPFHSSYAHSQSLDRLMNPLIDQYLYYLSRTNTPSGTTTQSTLQFSQAGASD
IRDQSRNWLPGPCYRQQRVSKTSADNNNSEYSWTGATKYHLNGRDSLVNPGPAMASHKDDEEKFFPQ
SGVLIFGKQGSEKTNVDIEKVMITDEEEIRTTNPVATEQYGSVSTNLQRGNRQAATADVNTQGVLPG
MVWQDRDVYLQGPIWAKIPHTDGHFHPSPLMGGFGLKHPPPQILIKNTPVPANPSTTFSAAKFASFI
TQYSTGQVSVEIEWELQKENSKRWNPEIQYTSNYNKSVNVDFTVDTNGVYSEPRPIGTRYLTRNL*
LP3-19 amino acid sequence
(SEQ ID NO: 23)
MAADGYLPDWLEDTLSEGIRQWWKLKPGPPPPKPAERHKDDSRGLVLPGYKYLGPFNGLDKGEPVNE
ADAAALEHDKAYDRQLDSGDNPYLKYNHADAEFQERLQEDTSFGGNLGRAVFQAKKRVLEPLGLVEE
GAKTAPGKKRPVEQSPQEPDSSSGIGKTGQQPAKKRLNFGQTGDSESVPDPQPLGEPPATPAAVGPT
TMASGGGAPMADNNEGADGVGNSSGNWHCDSTWLGDRVITTSTRTWALPTYNNHLYKQISSQSGASN
DNHYFGYSTPWGYFDFNRFHCHFSPRDWQRLINNNWGFRPKRLNFKLFNIQVKEVTDNNGVKTIANN
LTSTVQVFTDSEYQLPYVLGSAHQGCLPPFPADVFMIPQYGYLTLNNGSQAVGRSSFYCLEYFPSQM
LRTGNNFTFSYTFEDVPFHSSYAHSQSLDRLMNPLIDQYLYYLSRTQTTGGTANTQTLGFSQGGPNT
MANQAKNWLPGPCYRQQRVSTTTGQNNNSNFAWTAGTKYHLNGRNSLANPGIAMATHKDDEERFFPS
NGILIFGKQNAARDNADYSDVMLTSEEEIKTTNPVATEEYGIVADNLQQQNTAPQIGTVNSQGALPG
MVWQNRDVYLQGPIWAKIPHTDGNFHPSPLMGGFGLKHPPPQILIKNTPVPADPPTTFNQSKLNSFI
TQYSTGQVSVEIEWELQKENSKRWNPEIQYTSNYYKSTSVDFAVNTEGVYSEPRPIGTRYLTRNL*
LP4-7 amino acid sequence
(SEQ ID NO: 24)
MAADGYLPDWLEDNLSEGIREWWDLKPGAPKPKANQQKQDDGRGLVLPGYKYLGPFNGLDKGEPVNA
ADAAALEHDKAYDRQLDSGDNPYLKYNHADAEFQERLKEDTSFGGNLGRAVFQAKKRLLEPLGLVEE
AAKTAPGKKRPVEQSPQEPDSSSGIGKTGQQPAKKRLNFGQTGDSESVPDPQPLGEPPATPAAVGPT
TMASGGGAPMADNNEGADGVGSSSGNWHCDSQWLGDRVITTSTRTWALPTYNNHLYKQISNSTSGGA
TNDNTYFGYSTPWGYFDFNRFHCHFSPRDWQRLINNNWGFRPKRLNFKLFNIQVKEVTDNNGVKTIA
NNLTSTVQVFTDSEYQLPYVLGSAHQGCLPPFPADVFMIPQYGYLTLNNGSQAVGRSSFYCLEYFPS
QMLRTGNNFQFTYTFEDVPFHSSYAHSQSLDRLMNPLIDQYLYYLSRTQTTGGTANTQTLGFSQGGP
NTMANQAKNWLPGPCYRQQRVSTTTGQNNNSNFAWTAGTKYHLNGRNSLANPGIAMASHKDDKERFF
PSNGILIFGKQNAARDNADYSDVMLTSEEEIKTTNPVATEEYGIVADNLQQQNTAPQIGTVNSQGAL
PGMVWQNRDVYLQGPIWAKIPHTDGNFHPSPLMGGFGLKHPPPQILIKNTPVPADPPTTFNQSKLNS
FITQYSTGQVSVEIEWELQKENSKRWNPEIQYTSNYYKSTSVDFAVNTEGVYSEPHPIGTRYLTRP
L*
LP4-15 amino acid sequence
(SEQ ID NO: 25)
MAADGYLPDWLEDNLSEGIREWWALKPGAPKPKANQQKQDDGRGLVLPGYKYLGPFNGLDKGEPVNA
ADAAALEHDKAYDRQLDSGDNPYLKYNHADAEFQERLKEDTSFGGNLGRAVFQAKKRVLEPLGLVEE
PVKTAPGKKRPVEHSPVEPDSSSGTGKAGQQPARKRLNFGQTGDADSVPDPQPLGQPPAAPSGLGTN
TMATGSGAPMADNNEGADGVGNSSGNWHCDSTWMGDRVITTSTRTWALPTYNNHLYKQISSQSGASN
DNHYFGYSTPWGYFDFNRFHCHFSPRDWQRLINNNWGFRPKRLNFKLFNIQVKEVTQNDGTTTIANN
LTSTVQVFTDSEYQLPYVLGSAHQGCLPPFPADVFMVPQYGYLTLNNGSQAVGRSSFYCLEYFPSQM
LRTGNNFTFSYTFEDVPFHSSYAHSQSLDRLMNPLIDQYLYYLSRTNTPSGTTTQSTLQFSQAGASD
IRDQSRNWLPGPCYRQQRVSKTSADNNNSEYSWTGATKYHLNGRDSLVNPGPAIASHKDDEEKFFPQ
SGVLIFGKQGSEKTNVDIEKVMITDEEEIRTTNPVATEQYGSVSTNLQRGNRQAATADVNTQGVLPG
MVWQDRDVYLQGPIWAKIPHTDGHFHPSPLMGGFGLKHPPPQILIKNTPVPANPSTTFSAAKFASFI
TQYSTGQVSVEIEWELQKENSKRWNPEIQYTSNYNKSVNVDFTVDTNGVYSEPRPIGTRYLTRNL*
LP4-19 amino acid sequence
(SEQ ID NO: 26)
MAADGYLPDWLEDNLSEGIREWWALKPGAPKPKANQQKQDDGRGLVLPGYKYLGPFNGLDKGEPVNA
ADAAALEHDKAYDQQLKAGDNPYLRYNHADAEFQERLQEDTSFGGNLGRAVFQAKKRVLEPLGLVEE
GAKTAPGKKRPVEQSPQEPDSSSGIGKTGQQPAKKRLNFGQTGDSESVPDPQPLGEPPATPAAVGPT
TMASGGGAPMADNNEGADGVGNSSGNWHCDSTWLGDRVITTSTRTWALPTYNNHLYKQISSASTGAS
NDNHYFGYSTPWGYFDFNRFHCHFSPRDWQRLINNNWGFRPKRLNFKLFNIQVKEVTDNNGVKTIAN
NLTSTVQVFTDSEYQLPYVLGSAHQGCLPPFPADVFMIPQYGYLTLNNGSQAVGRSSFYCLEYFPSQ
MLRTGNNFQFTYTFKDVPFHSSYAHSQSLDRLMNPLIDQYLYYLSRTQTTGGTANTQTLGFSQGGPN
TMANQAKNWLPGPCYRQQRVSTTTGQNNNSNFAWTAGTKYHLNGRNSLANPGIAMATHKDDEERFFP
SNGILIFGKQNAARDNADYSDVMLTSEEEIKTTNPVATEEYGIVADNLQQQNTAPQIGTVNSQGALP
GMVWQNRDVYLQGPIWAKIPHTDGNFHPSPLMGGFGLKHPPPQILIKNTPVPADPPTTFNQSKLNSF
ITQYSTGQVSVEIEWELQKENSKRWNPEIQYTSNYYKSTSVDFAVNTEGVYSEPRPIGTRYLTRPL*
LP4-21 amino acid sequence
(SEQ ID NO: 27)
MAADGYLPDWLEDTLSEGIRQWWKLKPGPPPPKPAERHKDDSRGLVLPGYKYLGPFNGLDKGEPVNA
ADAAALEHDKAYDQQLKAGDNPYLRYNHADAEFQERLQEDTSFGGNLGRAVFQAKKRVLEPFGLVEE
GAKTAPGKKRPVEQSPQEPDSSSGIGKTGQQPAKKRLNFGQTGDSESVPDPQPLGEPPATPAAVGPT
TMASGGGAPMADNNEGADGVGNSSGNWHCDSTWLGDRVITTSTRTWALPTYNNHLYKQISSASTGAS
NDNHYFGYSTPWGYFDFNRFHCHFSPRDWQRLINNNWGFRPKRLNFKLFNIQVKEVTDNNGVKTIAN
NLTSTVQVFTDSEYQLPYVLGSAHQGCLPPFPADVFMIPQYGYLTLNNGSQAVGRSSFYCLEYFPSQ
MLRTGNNFQFTYTFEDVPFHSSYAHSQSLDRLMNPLIDQYLYYLSRTQTTGGTANTQTLGFSQGGPN
TMANQAKNWLPGPCYRQQRVSTTTGQNNNSNFAWTAGTKYHLNGRNSLANPGIAMASHKDDKERFFP
SNGILIFGKQNAARDNADYSDVMLTSEEEIKTTNPVATEEYGIVADNLQQQNTAPQIGTVNSQGALP
GMVWQNRDVYLQGPIWAKIPHTDGNFHPSPLMGGFGLKHPPPQILIKNTPVPADPPTTFNQSKLNSF
ITQYSTGQVSVEIEWELQKENSKRWNPEIQYTSNYYKSTSVDFAVNTEGVYSEPHPIGTRYLTRNL*
LP4-24 amino acid sequence
(SEQ ID NO: 28)
MAADGYLPDWLEDTLSEGIRQWWKLKPGPPPPKPAERHKDDSRGLVLPGYKYLGPFNGLDKGEPVNA
ADAAALEHDKAYDRQLKAGDNPYLRYNHADAEFQERLKEDTSFGGNLGRAVFQAKKRVLEPFGLVEE
GAKTAPGKKRPVEQSPQEPDSSSGIGKTGQQPAKKRLNFGQTGDTESVPDPQPIGEPPAAPSGVGSL
TMASGGGAPVADNNEGADGVGSSSGNWHCDSQWLGDRVITTSTRTWALPTYNNHLYKQISSASTGAS
NDNHYFGYSTPWGYFDFNRFHCHFSPRDWQRLINNNWGFRPKRLNFKLFNIQVKEVTDNNGVKTIAN
NLTSTVQVFTDSEYQLPYVLGSAHQGCLPPFPADVFMIPQYGYLTLNNGSQAVGRSSFYCLEYFPSQ
MLRTGNNFQFTYTFEDVPFHSSYAHSQSLDRLMNPLIDQYLYYLSRTQTTGGTANTQTLGFSQGGPN
TMANQAKNWLPGPCYRQQRVSTTTGQNNNSNFAWTAGTKYHLNGRNSLANPGIAMASHKDDKERFFP
SNGILIFGKQNAARDNADYSDVMLTSEEEIKTTNPVATEEYGIVADNLQQQNTAPQIGTVNSQGALP
GMVWQNRDVYLQGPIWAKIPHTDGNFHPSPLMGGFGLKHPPPQILIKNTPVPADPPTTFNQSKLNSF
ITQYSTGQVSVEIEWELQKENSKRWNPEIQYTSNYYKSTSVDFAVNTEGVYSEPRPIGTRYLTRNL*
AAV1 VR1 loop
(SEQ ID NO: 29)
QISSASTGASNDNH
AAV2 VR1 loop
(SEQ ID NO: 30)
QISSQSGASNDNHY
AAV3b VR1 loop
(SEQ ID NO: 31)
QISSQSGASNDNHY
AAV4 VR1 loop
(SEQ ID NO: 32)
RLGESLQSNTY
AAV5 VR1 loop
(SEQ ID NO: 33)
SVDGSNANAY
AAV6 VR1 loop
(SEQ ID NO: 34)
QISSASTGASNDNH
AAV7 VR1 loop
(SEQ ID NO: 35)
QISSETAGSTNDNT
AAV8 VR1 loop
(SEQ ID NO: 36)
QISNGTSGGATNDNT
AAV9 VR1 loop
(SEQ ID NO: 37)
QISNSTSGGSSNDNA
AAV10 VR1 loop
(SEQ ID NO: 38)
QISNGTSGGSTNDNT
AAV11 VR1 loop
(SEQ ID NO: 39)
LRLGTTSSSNTY
AAV12 VR1 loop
(SEQ ID NO: 40)
LRIGTTANSNT
AAV13 VR1 loop
(SEQ ID NO: 41)
QISSQSGATNDNHY

1. A nucleic acid encoding an AAV capsid protein, the nucleic acid comprising an AAV capsid protein coding sequence that is at least 90% identical to:
(a) the nucleotide sequence of any one of SEQ ID NOS:1-14; or
(b) a nucleotide sequence encoding any one of SEQ ID NOS:15-28.

2-14. (canceled)

15. An AAV capsid protein comprising an amino acid sequence at least 90% identical to any one of SEQ ID NOS:15-28.

16-18. (canceled)

19. The AAV capsid protein of claim 15, comprising the amino acid sequence of any one of SEQ ID NOS: 15-28.

20. An AAV capsid protein comprising one or more of the following mutations:
a) Q105K;
b) A135G;
c) T179S;
d) I188L;
e) S200P;
f) L201N;
g) D348E;
h) E360Q;
i) D383N;
j) E417K;
k) R459T;
l) H510N;
m) M523I;
n) T526S;
o) P724H;
p) R725H;
q) N735P;
based on the AAV8 capsid protein sequence or the corresponding residue in another AAV capsid protein.

21. An AAV capsid protein comprising one or more mutations in the variable region 1 (VR1) loop and or a chimeric capsid protein in which the VR1 loop has been replaced by a VR1 loop from a capsid protein of a different AAV serotype, wherein an AAV particle comprising the encoded capsid protein has decreased susceptibility to neutralizing antibodies when administered to a subject relative to an AAV particle comprising a wild-type capsid protein, and wherein the VR1 loop corresponds to amino acid residues QISNGTSGGATNDNT (SEQ ID NO:36) in the AAV8 capsid protein and the corresponding amino acids in other serotypes.

22. The AAV capsid protein of claim 15, wherein the capsid protein further comprises an E531K mutation based on the AAV8 capsid protein sequence or the corresponding residue in another AAV capsid protein.

23. The AAV capsid protein of claim 15 covalently linked, bound to, or encapsidating a compound selected from the group consisting of a DNA molecule, an RNA molecule, a polypeptide, a carbohydrate, a lipid, and a small organic molecule.

24. An AAV particle comprising:
an AAV vector genome; and
the AAV capsid of claim 15, wherein the AAV capsid encapsidates the AAV vector genome.

25. The AAV particle of claim 24, wherein the AAV vector genome comprises a heterologous nucleic acid.

26. The AAV particle of claim 25, wherein the heterologous nucleic acid encodes an antisense RNA, microRNA, or RNAi.

27. The AAV particle of claim 25, wherein the heterologous nucleic acid encodes a polypeptide, e.g., a therapeutic polypeptide or a reporter protein.

28-29. (canceled)

30. The AAV particle of claim 24, wherein the heterologous nucleic acid is operably linked to a RNA polymerase II-based or RNA polymerase III-based promoter, e.g., a constitutive promoter or an inducible promoter.

31. The AAV particle of claim 24, wherein the heterologous nucleic acid is operably linked to a liver-specific or liver-preferred promoter.

32. The AAV particle of claim 31, wherein the liver-specific or liver-preferred promoter is a promoter from apolipoprotein AII, albumin, alpha 1-antitrypsin, thyroxine-binding globulin, cytochrome P450 CYP3A4, or microRNA122 or a synthetic liver-specific regulatory sequence.

33. A method of producing a recombinant AAV particle comprising an AAV capsid, the method comprising:
providing a cell in vitro with a nucleic acid according to claim 1, an AAV rep coding sequence, an AAV vector genome comprising a heterologous nucleic acid, and helper functions for generating a productive AAV infection; and
allowing assembly of the recombinant AAV particle comprising the AAV capsid and encapsidating the AAV vector genome.

34. (canceled)

35. A pharmaceutical formulation comprising the AAV particle of claim 24 in a pharmaceutically acceptable carrier.

36. A method of delivering a nucleic acid of interest to a cell, the method comprising contacting the cell with the AAV particle of claim 24.

37. (canceled)

38. A method of delivering a nucleic acid of interest to a cell in a mammalian subject, the method comprising:
administering an effective amount of the AAV particle of claim 24 to a mammalian subject, thereby delivering the nucleic acid of interest to a cell in the mammalian subject.

39-42. (canceled)

43. A method of treating a disorder in a mammalian subject in need thereof, wherein the disorder is treatable by expressing a product in the subject, the method comprising administering a therapeutically effective amount of the AAV particle of claim 24 to a mammalian subject, wherein the product is expressed, thereby treating the disorder.

44-46. (canceled)

47. A method of decreasing the susceptibility of an AAV particle to neutralizing antibodies when administered to a subject, comprising preparing the AAV particle with the capsid protein of claim 15.
